(19) United States
(12) Patent Application Publication
(10) Pub. No.: US 2026/0315436 A1
(43) Pub. Date: Oct. 15, 2026

(54) METHOD OF TREATING DISORDERS WITH SURF1 MALFUNCTION

(71) Applicants: Cure Mito Foundation, McKinney, TX (US); Heinrich-Heine-Universität Düsseldorf, Düsseldorf (DE)

(72) Inventors: Ethan Perlstein, McKinney, TX (US); Justin Donnelly, McKinney, TX (US); Mathura Thevandavakkam, McKinney, TX (US); Alessandro Prigione, Düsseldorf (DE); Carmen Maria Menacho Pando, Düsseldorf (DE)

(21) Appl. No.: 19/165,961

(22) PCT Filed: Mar. 17, 2024
(86) PCT No.: PCT/US2024/020338

§ 371 Date: Sep. 16, 2025

Related U.S. Application Data
(60) Provisional application No. 63/491,017, filed on Mar. 17, 2023.

(51) Int. Cl. A61K 31/4178; A61K 31/4196; A61K 31/4439; A61K 31/496; A61K 31/506; A61K 45/06

(52) U.S. Cl. CPC A61K 31/4178; A61K 31/4196; A61K 31/4439; A61K 31/496; A61K 31/506; A61K 45/06

(57) ABSTRACT

A method of restoring the functionality of complex IV in mitochondrial disorders including Leigh Syndrome is disclosed utilizing drug repurposing. One or more repurposed drugs can be used to treat Leigh Syndrome by administering to a patient a therapeutically effective amount of the one or more repurposed drugs. Such repurposed drugs are capable of rescuing a defective SURF1 gene in the patient.

### CROSS-REFERENCE TO RELATED APPLICATION

This application claims the benefit of U.S. Provisional Patent Application No. 63/491,017, filed Mar. 17, 2023, which is incorporated by reference herein in its entirety.

### TECHNICAL FIELD

This disclosure is generally related to treatment of mitochondrial disorders. Specifically, this disclosure is related to the treatment of Leigh Syndrome.

### BACKGROUND OF THE INVENTION

Mitochondrial diseases affect 1 in 5,000 live births around the world. They are caused by inherited or de novo mutations in over 350 nuclear-encoded and mtDNA-encoded genes.

Leigh syndrome (SURF1-LS) is a rare untreatable neurologic mitochondrial disorder affecting children. Leigh Syndrome is caused by a problem with oxidative phosphorylation.

Cytochrome c oxidase (COX) (known as complex IV in the oxidation phosphorylation (oxphos)/electron transport pathway). COX catalyzes the oxidation of cytochrome c and the reduction of oxygen to water. Mammalian COX is made up of fourteen subunits (including NDUFA4). Eleven of the subunits are nuclear-encoded (COX4I1 (or COX4I2), COX5A, COX5B, COX6A1 (or COX6A2), COX6B1 (or COX6B2), COX6C, COX7A2 (or COX7A1), COX7B, COX7C, COX8A and NDUFA4) and three subunits are mitochondrial-encoded (MT-CO1, MT-CO2, and MT-CO3). Leigh syndrome may be due to a deficiency in any of the complexes in the oxphos pathway but the functional absence of SURF1 is a common cause of Leigh Syndrome. Homozygous or compound heterozygous mutation in the SURF1 gene causes mitochondrial complex IV (cytochrome c oxidase) deficiency-1 (MC4DN1), a autosomal recessive metabolic disorder.

SURF1 is a nuclear gene that encodes a 300 amino acid mitochondrial protein, present in the mitochondrial inner membrane, that aids in COX assembly. The SURF1 mitochondrial inner membrane protein is required for Complex IV assembly and activity. Most SURF1 pathogenic loss-of-function mutations result in a truncated or absent protein. The most common recurring SURF1 mutation, which mirrors the effect of SURF1 whole-gene deletion, is c.312_320del10insAT, resulting in Leu105X. It is thought that SURF1 is involved in heme insertion into complex IV.

It would be advantageous to have a method of restoring the functionality of complex IV in mitochondrial disorders including Leigh Syndrome.

### SUMMARY OF THE INVENTION

An embodiment of the disclosure is a method of treating Leigh Syndrome comprising administering to a patient in need thereof a therapeutically effective amount of one or more drugs, wherein the one or more repurposed drugs is an antifungal, azole antifungal, chlorinated phenol, echinocandin, lipophilic antifungal, statin, lysophospholipid, anionic surfactant, cationic surfactant, lipophilic antioxidant supplement, non-steroid anti-inflammatory drug, steroid, or other compound capable of treating Leigh Syndrome.

In an embodiment, the one or more drugs is sertaconazole.

In an embodiment, the one or more drugs are haloprogin, dichlorophene, atorvastatin, caspofungin, micafungin, miltefosine, sodium tetradecyl sulfate, or telapravir.

In an embodiment, the one or more drugs are clotrimazole, bifonazole, climbazole, fluconazole, voriconazole, tioconazole, sertaconazole, econazole, miconazole, butoconazole, oxiconazole, itraconazole, posaconazole, ketoconazole, terconazole, fenbendazole, terbinafine, amorolfine, VT-1161, haloprogin, dichlorophene, triclosan, bithinol, hexachlorophene, chloroxine, nitroxoline, benzbromarone, anidulafungin, caspofungin, micafungin, cerivastin, atorvastatin, fluvastatin, pitastatin, rosuvastatin, miltefosine, benzethonium chloride, sodium tetrdecyl sulfate, suloctidil, hexetidine, alexidine, ascorbyl palmitate, menadione, hexylresorcinol, flufenamic acid, tolfenamic acid, prasterone, pregnenolone, norgestimate, telapravir, pargyline, erythromycin, diclazuril, gliquidone, or nitazoxanide.

In an embodiment, the one or more drugs are clotrimazole, bifonazole, climbazole, fluconazole, voriconazole, tioconazole, sertaconazole, econazole, miconazole, butoconazole, oxiconazole, itraconazole, posaconazole, ketoconazole, terconazole, fenbendazole, terbinafine, amoroltine, or VT-1161.

In an embodiment, the one or more drugs are haloprogin, dichlorophene, triclosan, bithinol, hexachlorophene, chloroxine, nitroxoline, or benzbromarone.

In an embodiment, the one or more drugs are anidulafungin, caspofungin, or micafungin.

In an embodiment, the one or more drugs are cerivastin, atorvastatin, fluvastatin, pitastatin, or rosuvastatin.

In an embodiment, the one or more drugs are miltefosine or benzethonium chloride.

In an embodiment, the one or more drugs are sodium tetrdecyl sulfate or suloctidil.

In an embodiment, the one or more drugs are hexetidine or alexidine.

In an embodiment, the one or more drugs are ascorbyl palmitate, menadione, or hexylresorcinol.

In an embodiment, the one or more drugs are flufenamic acid or tolfenamic acid.

In an embodiment, the one or more drugs are prasterone, pregnenolone, or norgestimate.

In an embodiment, the one or more drugs are telapravir, pargyline, erythromycin, diclazuril, gliquidone, or nitazoxanide.

In an embodiment, the one or more drugs are posacanazole, sertaconazole, glabridin, zinglibroside (ginsenoside Z R1), AM281, cysteamine bitrartate, PF-06447475, losmapimod, telepravir, miltefosine, incyclinide, poziotinib, and methylene blue.

An embodiment of the disclosure is a method of treating Leigh Syndrome comprising administering to a patient in need thereof a therapeutically effective amount of one or more drugs capable of rescuing a defective SURF1 gene, wherein the one or more repurposed drugs is an antifungal, azole antifungal, chlorinated phenol, echinocandin, lipophilic azole antifungal, statin, lysophospholipid, anionic surfactant, cationic surfactant, lipophilic antioxidant supplement, non-steroid anti-inflammatory drug, steroid, or other compound capable of treating Leigh Syndrome.

In an embodiment, the one or more drugs is sertaconazole.

In an embodiment, the one or more drugs are haloprogin, dichlorophene, atorvastatin, caspofungin, micafungin, miltefosine, sodium tetradecyl sulfate, or telapravir.

In an embodiment, the one or more drugs are clotrimazole, bifonazole, climbazole, fluconazole, voriconazole, tioconazole, sertaconazole, econazole, miconazole, butoconazole, oxiconazole, itraconazole, posaconazole, ketoconazole, terconazole, fenbendazole, terbinafine, amoroltine, VT-1161, haloprogin, dichlorophene, triclosan, bithinol, hexachlorophene, chloroxine, nitroxoline, benzbromarone, anidulafungin, caspofungin, micafungin, cerivastin, atorvastatin, fluvastatin, pitastatin, rosuvastatin, miltefosine, benzethonium chloride, sodium tetrdecyl sulfate, suloctidil, hexetidine, alexidine, ascorbyl palmitate, menadione, hexylresorcinol, flufenamic acid, tolfenamic acid, prasterone, pregnenolone, norgestimate, telapravir, pargyline, erythromycin, diclazuril, gliquidone, or nitazoxanide.

In an embodiment, the one or more drugs are posacanazole, sertaconazole, glabridin, zinglibroside (ginsenoside Z R1), AM281, cysteamine bitrartate, PF-06447475, losmapimod, telepravir, miltefosine, incyclinide, poziotinib, and methylene blue.

An embodiment of the disclosure is a method of restoring the functionality of complex IV in mitochondrial disorders comprising administering to a patient in need thereof a therapeutically effective amount of one or more drugs, wherein the one or more drugs are an antifungal, azole antifungal, chlorinated phenol, echinocandin, lipophilic azole antifungal, statin, lysophospholipid, anionic surfactant, cationic surfactant, lipophilic antioxidant supplement, non-steroid anti-inflammatory drug, steroid, or other compound capable of restoring the functionality of complex IV in mitochondrial disorders.

In an embodiment, the one or more drugs is sertaconazole.

In an embodiment, the one or more drugs are haloprogin, dichlorophene, atorvastatin, caspofungin, micafungin, miltefosine, sodium tetradecyl sulfate, or telapravir.

In an embodiment, the one or more drugs are clotrimazole, bifonazole, climbazole, fluconazole, voriconazole, tioconazole, sertaconazole, econazole, miconazole, butoconazole, oxiconazole, itraconazole, posaconazole, ketoconazole, terconazole, fenbendazole, terbinafine, amorolfine, VT-1161, haloprogin, dichlorophene, triclosan, bithinol, hexachlorophene, chloroxine, nitroxoline, benzbromarone, anidulafungin, caspofungin, micafungin, cerivastin, atorvastatin, fluvastatin, pitastatin, rosuvastatin, miltefosine, benzethonium chloride, sodium tetrdecyl sulfate, suloctidil, hexetidine, alexidine, ascorbyl palmitate, menadione, hexylresorcinol, flufenamic acid, tolfenamic acid, prasterone, pregnenolone, norgestimate, telapravir, pargyline, erythromycin, diclazuril, gliquidone, or nitazoxanide.

In an embodiment, the one or more drugs are posacanazole, sertaconazole, glabridin, zinglibroside (ginsenoside Z R1), AM281, cysteamine bitrartate, PF-06447475, losmapimod, telepravir, miltefosine, incyclinide, poziotinib, and methylene blue.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE FIGURES

The present technology will be better understood on reading the following detailed descriptions of non-limiting embodiments thereof, and on examining the accompanying drawings, in which:

FIG. 1 depicts COX activation via “active site chaperones” via tyrosine and/or histidine interactions.

FIG. 2 depicts structures showing the pharmacological evolution from clomitrazole (imidazole) to fluconazole to voriconazole to itraconazole (triazoles) to experimental (tetrazole) VT-1161.

FIG. 3 depicts mitochondrial localization (amine to hydrophobicity ratio).

FIG. 4 depicts a structure of fluconazole bound to CYP51 heme-dependent cytochrome P450 sterol demethylase.

FIG. 5 depicts structures of chlorinated phenols comprising haloprogin, dichlorophene, parachlorophenol, triclosan, bithinol, hexachlorophene, chloroxine, nitroxoline, benzbromarone, and amiodarone.

FIG. 6 depicts structures of echinocandins comprising anidulafungin, caspofungin, and micafungin.

FIG. 7 depicts a structural overview of echinocandin biosynthesis (Hüttel, Z. Naturforsch, 2017; 72(1-2)c:1-20).

FIG. 8 depicts structures of azole antifungals comprising clotrimazole, bifonazole, climbazole, fluconazole, voriconazole, tioconazole, sertaconazole, econazole, miconazole, butoconazole, oxiconazole, enilconazole, metronidazole, tinidazole, and chlormidazole.

FIG. 9 depicts structures of lipophilic azole antifungals comprising posaconazole, itraconazole, ketoconazole, and terconazole.

FIG. 10 depicts structures of antifungals comprising fenbendazole, terbinafine, amorolfine, oxfendazole, and naftidine.

FIG. 11 depicts structures of statins comprising cerivastin, atorvastatin, pravastatin, fluvastatin, pitastatin, and rosuvastatin.

FIG. 12 depicts structures of inactive statins comprising pravastatin, simvastatin, mevastatin, and lovastatin.

FIG. 13 depicts a structures of lysophospholipid comprising miltefosine, methacholine, benzethonium chloride, and oleic acid.

FIG. 14 depicts structures of anionic surfactants comprising sodium tetrdecyl sulfate and suloctidil.

FIG. 15 depicts structures of cationic surfactants comprising hexetidine and alexidine.

FIG. 16 depicts structures of lipophilic antioxidant supplements comprising ascorbyl palmitate, menadione, and hexylresorcinol.

FIG. 17 depicts structures of non-steroidal anti-inflammatory drugs (NSAIDS) comprising flufenamic acid, and tolfenamic acid.

FIG. 18 depicts structures of steroids comprising prasterone, pregnenolone, and norgestimate.

FIG. 19 depicts other compounds comprising telapravir, pargyline, chlorgyline, selegiline, erythromycin, diclazuril, gliquidone, and nitazoxanide.

FIG. 20 depicts primary drug repurposing data. Haploid and diploid SURF1 deficient mutants were screened. The positive control is a wildtype (unmutated) yeast strain.

FIG. 21 depicts graph of results of a Bac-TiterGlo luminescence assay for PY1 WT haploid, PY3 WT diploid, PY15 shy1Δ haploid, and PY17 shy1Δ diploid.

FIG. 22 depicts results of a Z′ optimization experiment for WT versus the haploid shy1Δ (PY15).

FIG. 23 depicts results of a Z′ optimization experiment for WT versus the diploid shy1Δ (PY17).

FIG. 24A and FIG. 24B depict intensity b-scores (equivalent to a Z-score) for the A) haploid shy1Δ knockout mutant and the B) diploid shy1Δ knockout mutant.

FIG. 25A and FIG. 25B depict the full Pharmakon dataset for the A) haploid shy1Δ knockout mutant and the B) diploid homozygous shy1Δ knockout, showing the ~2,500 library compounds (red triangles) plotted along with the positive controls (cyan triangles) and the negative controls (green triangles) that are obscured by the baseline of inactive compounds at 0 intensity activation (cell number). Red triangles=hits with the larger the triangles, the more statistically significant the result.

FIG. 26 depicts the haploid b-score rank.

FIG. 27 depicts the diploid b-score rank.

FIGS. 28A, 28B, and 28C depict the impact of the selected hit compounds on neurite outgrowth capacity.

FIGS. 29A, 29B, 29C, and 29D depict that Sertaconazole reduced the aberrant release of lactate in the media, increased the amount of TH-positive dopaminergic neurons, and rescued the overall neuronal branching organization.

FIGS. 30A and 30B depict a Mitochondrial “Stress Test” Overview.

FIGS. 31A, 31B, 31C, 31D, and 31E depict the compound treatment plate controls.

FIGS. 32A, 32B, 32C, 32D, 32E, 32F, and 32G depict that treatment with losmapimod for 48 hours displayed 12-15% increases in respiration in ATP-linked and maximal respiration in both glucose and galactose-cultured cells. There was also a decrease in ATP production through glycolysis.

FIGS. 33A, 33B, and 33C depict that treatment with PF-06447475 for 48 hours did not result in an increase in maximal respiration in either glucose or galactose conditions.

FIGS. 34A, 34B, and 34C depict that treatment with Ginsenoside Z R1 (Zinglibroside) for 48 hours did not result in changes to maximal respiration in either glucose or galactose conditions.

FIGS. 35A, 35B, and 35C depict that treatment with Glabridin for 48 hours did not result in major changes to respiration in either glucose or galactose conditions.

FIGS. 36A, 36B, 36C, and 36D depict that treatment with incyclinide for 48 hours showed some increase to basal respiration and ATP-linked respiration in galactose-cultured cells.

FIGS. 37A, 37B, and 37C depict that treatment with miltefosine for 48 hours did not result in major changes to respiration in either glucose or galactose conditions.

FIGS. 38A, 38B, and 38C depict that treatment with posaconazole for 48 hours did not result in major changes to respiration in either glucose or galactose conditions. At higher concentrations, it decreased respiration.

FIGS. 39A, 39B, and 39C depict that treatment with sertaconazole for 48 hours did not result in major changes to respiration in either glucose or galactose conditions.

FIGS. 40A, 40B, and 40C depict that treatment with telaprevir for 48 hours did not result in major changes to respiration in either glucose or galactose conditions.

FIGS. 41A, 41B, and 41C depict that treatment with poziotinib for 48 hours did not result in major changes to respiration in either glucose or galactose conditions.

FIGS. 42A, 42B, and 42C depict that treatment with AM281 for 48 hours did not result in major changes to respiration in either glucose or galactose conditions.

FIGS. 43A, 43B, and 43C depict that treatment with the triple combination of compounds did not alter oxygen consumption in GM28010 cells.

FIGS. 44A, 44B, and 44C depict that treatment with the double combination of losmapimod and ginsenoside slightly increased respiration and ATP production rate through OXPHOS while decreasing ATP production through glycolysis.

FIGS. 45A, 45B, and 45C depict that treatment with the double combination of ginsenoside and PF-06447475 slightly increased respiration and ATP production rate through OXPHOS while maintaining total ATP production rates.

FIGS. 46A, 46B, and 46C depict that treatment with the double combination of losmapimod and PF-06447475 slightly increased respiration and ATP production rate through OXPHOS while maintaining total ATP production rates.

FIGS. 47A, 47B, and 47C depict that treatment with cysteamine bitartrate did not alter oxygen consumption rates in GM28010 cells.

FIGS. 48A, 48B, and 48C depict that treatment with methylene blue decreased oxygen consumption rates in GM28010 cells while maintain ATP production rates.

FIG. 49A and FIG. 49B depict the compound treatment plate controls.

FIGS. 50A, 50B, and 50C depict that treatment with losmapimod for 48 hours displayed increased in ATP-linked respiration in glucose-cultured cells.

FIGS. 51A, 51B, and 51C depict that treatment with cysteamine bitartrate for 48 hours increased basal respiration and ATP-Linked respiration in a dose-dependent manner in both glucose and galactose culture conditions decrease ATP production through glycolysis.

FIGS. 52A, 52B, and 52C depict that treatment with Ginsenoside Z R1 (Zinglibroside) for 48 hours did not result in changes to maximal respiration in either glucose or galactose conditions.

FIGS. 53A, 53B, and 53C depict that treatment with telaprevir for 48 hours did not result in major changes to respiration in galactose conditions.

FIGS. 54A, 54B, and 54C depict that treatment with glabridin for 48 hours decreased maximal respiration in both glucose or galactose conditions.

FIGS. 55A, 55B, and 55C depict that treatment with incyclinide for 48 hours showed toxicity in glucose-cultured cells with a dose-dependent decrease in all respiratory parameters measured.

FIGS. 56A, 56B, and 56C depict that treatment with miltefosine for 48 hours did not result in major changes to respiration in either glucose or galactose conditions.

FIGS. 57A, 57B, and 57C depict that treatment with PF-06447475 for 48 hours did not result increase maximal respiration in either glucose or galactose conditions.

FIGS. 58A, 58B, and 58C depict that treatment with posaconazole for 48 hours decreased respiration in both glucose or galactose conditions.

FIGS. 59A, 59B, and 59C depict that treatment with Sertaconazole for 48 hours decreased respiration in both glucose and galactose and increased ATP production through glycolysis at higher concentrations.

FIGS. 60A, 60B, and 60C depict that treatment with AM281 for 48 hours increased ATP production rates but also showed a dose-dependent decreased maximal respiration.

FIG. 61A and FIG. 61B depict compound treatment controls.

FIGS. 62A, 62B, 62C, 62D, and 62E depict the controls on the compound treatment plates.

FIGS. 63A, 63B, 63C, 63D, 63E, and 63F depict that treatment with posaconazole for 48 hours did not result in major changes to respiration in either glucose or galactose conditions.

FIGS. 64A, 64B, 64C, 64D, 64E, and 64F depict that treatment with Sertaconazole for 48 hours did not result in major changes to respiration in either glucose or galactose conditions.

FIGS. 65A, 65B, 65C, 65D, 65E, and 65F depict that treatment with glabridin for 48 hours did not result in major changes to respiration in either glucose or galactose conditions.

FIGS. 66A, 66B, 66C, 66D, 66E, and 66F depict that treatment with zinglibroside for 48 hours did not result in changes to maximal respiration in either glucose or galactose conditions.

FIGS. 67A, 67B, 67C, 67D, 67E, and 67F depict that treatment with AM281 for 48 hours did not result in major changes to respiration in either glucose or galactose conditions.

FIGS. 68A, 68B, 68C, 68D, 68E, and 68F depict that treatment with cysteamine bitartrate did not alter oxygen consumption rates in GM28010 cells.

FIGS. 69A, 69B, 69C, 69D, 69E, and 69F depict that treatment with PF-06447475 for 48 hours did not result increase maximal respiration in either glucose or galactose conditions.

FIGS. 70A, 70B, 70C, 70D, 70E, and 70F depict that treatment with losmapimod for 48 hours displayed 12-15% increases in respiration in ATP-linked and maximal respiration in both glucose and galactose-cultured cells.

FIGS. 71A, 71B, 71C, 71D, 71E, 71F, and 71G depict that treatment with losmapimod for 48 hours displayed 12-15% increases in respiration in ATP-linked and maximal respiration in both glucose and galactose-cultured cells.

FIGS. 72A, 72B, 72C, 72D, 72E, and 72F depict that treatment with telaprevir for 48 hours did not result in major changes to respiration in either glucose or galactose conditions.

FIGS. 73A, 73B, 73C, 73D, 73E, and 73F depict that treatment with miltefosine for 48 hours did not result in major changes to respiration in either glucose or galactose conditions.

FIGS. 74A, 74B, 74C, 74D, 74E, 74F, and 74G depict that treatment with incyclinide for 48 hours showed some increase to basal respiration and ATP-linked respiration in galactose-cultured cells.

FIGS. 75A, 75B, and 75C depict that treatment with poziotinib for 48 hours did not result in major changes to respiration in either glucose or galactose conditions.

### DETAILED DESCRIPTION

Unless defined otherwise, all technical and scientific terms used herein have the same meaning as is commonly understood by one of skill in the art to which this disclosure belongs.

When introducing elements of various embodiments of the present invention, the articles “a,” “an,” “the,” and “said” are intended to mean that there are one or more of the elements. The terms “comprising,” “including,” and “having” are intended to be inclusive and mean that there may be additional elements other than the listed elements. Any examples of operating parameters and/or environmental conditions are not exclusive of other parameters/conditions of the disclosed embodiments. Additionally, it should be understood that references to “one embodiment,” “an embodiment,” “certain embodiments,” or “other embodiments” of the present invention are not intended to be interpreted as excluding the existence of additional embodiments that also incorporate the recited features. Furthermore, reference to terms such as “above,” “below,” “upper,” “lower,” “side,” “front,” “back,” or other terms regarding orientation are made with reference to the illustrated embodiments and are not intended to be limiting or exclude other orientations.

Finding a new use for an approved drug is called repurposing. Finding a repurposed drug that is active in treating mitochondrial diseases is attractive because of the reduced safety risks, low costs and fast timelines to a clinic-ready therapy or combination of therapies. Various off label drugs have been tested in yeast to determine if they rescue the SURF1 mutants, with the goal that these drugs could be repurposed as a treatment for mitochondrial disorders.

An aim of the present disclosure is to identify SURF1 Leigh Syndrome drug repurposing clinical candidates using a rapid turnaround, multi-species phenotypic screening approach that starts with the development and phenotypic characterization of genetically personalized yeast strains—“patient avatars”—and culminates in a drug repurposing screen, a process previously published for PMM2-CDG in Lao et al., 2019 Feb 7; 9(2):413423. Yeast strains expressing disease-causing hypomorphic variants exhibit growth defects that are directly proportional to the residual protein function. Slow growth can be rescued in a pathway-agnostic high-throughput chemical suppressor screen. As in the case of PMM2-CDG, the SURF1 yeast ortholog, gene identifier SHY1, is essential undergrowth conditions that require functional mitochondria, i.e., respiration. The yeast species Yarrowia lipolytica was used for the screen since it is an obligate aerobe (requires oxygen to grow), contains a fully functional electron transport chain, and has already been prototyped in a drug repurposing screen (Perlstein 2020). A 384-well-plate growth-based assay with appropriate positive and negative controls was designed and optimized for a drug repurposing screen (~5,000 compounds). The drug repurposing screen for mitochondrial diseases involving complex IV deficiency is described herein. A general yeast screening method was discussed in Perlstein, Drug repurposing for mitochondrial diseases using a pharmacological model of complex I deficiency in the yeast Yarrowia lipolytica, bioRxiv, (2020), see https://www.biorxiv.org/content/10.1101/2020.01.08.899666v1.

Yeast engineered to express the most common SURF1 pathogenic variant are the fastest turnaround, most cost-effective, and most representative disease models in which to conduct drug repurposing screens. The genome of Yarrowia lipolytica contains one SURF homolog called SHY1. Previous mutational studies in the baker's yeast Saccharomyces cerevisiae (Barrientos et al., EMBO J. 2002; 21:43-52; Reinhold et al., Human Molecular Genetics, 2011, 20(12): 2379-2393) studied SURF1 patient-derived mutations in comparison to a SURF1 whole-gene deletion, establishing phenotypic equivalence between the SURF1 patient mutations and complete loss-of-function of SURF1. A functionally inactive SHY1 yeast model can be generated using the G137E missense mutation (Bestwick et al., Mol Cell Biol. 2010; 30:172-185).

Yeast cells completely lacking SURF1 are unable to grow in conditions that require their mitochondria to produce energy. Knocking out the SURF1 ortholog in yeast—SHY1—results in loss of the ability of the knockout mutant to grow on a non-fermentable carbon source, e.g., lactate or glycerol, that requires functional mitochondria. A shy1Δ knockout mutant is incapable of growing on YEPG, or yeast media spiked with ethanol and glycerol as the sole non-fermentable carbon sources.

A pilot screen of ~2,500 known drugs was performed and a unique hit signature was identified. A drug repurposing screen was performed.

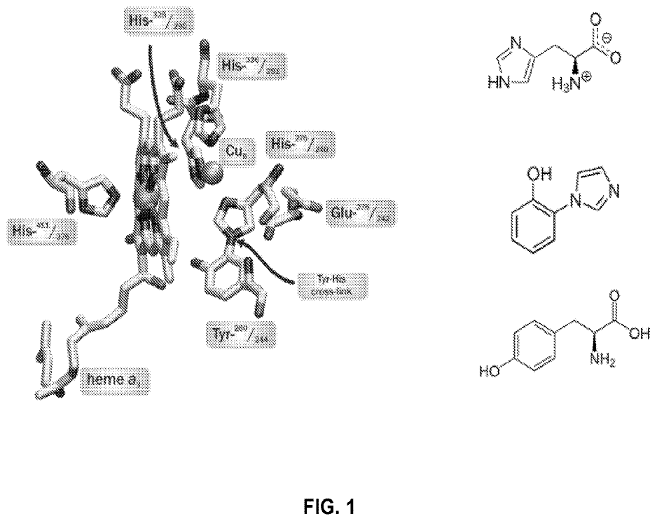

FIG. 1 depicts COX activation via “active site chaperones” via tyrosine and/or histidine interactions.

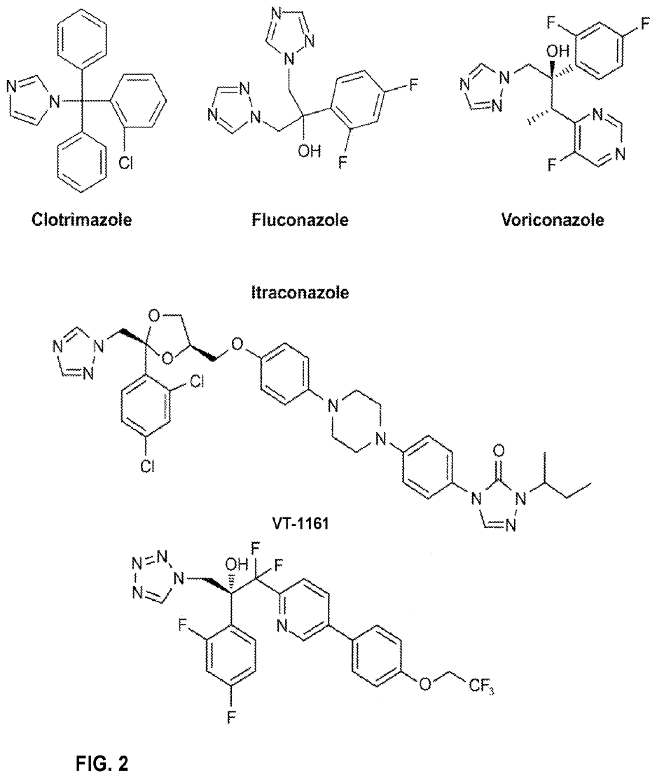

FIG. 2 depicts structures showing the pharmacological evolution from clomitrazole (imidazole) to fluconazole to voriconazole to itraconazole (triazoles) to experimental (tetrazole) VT-1161.

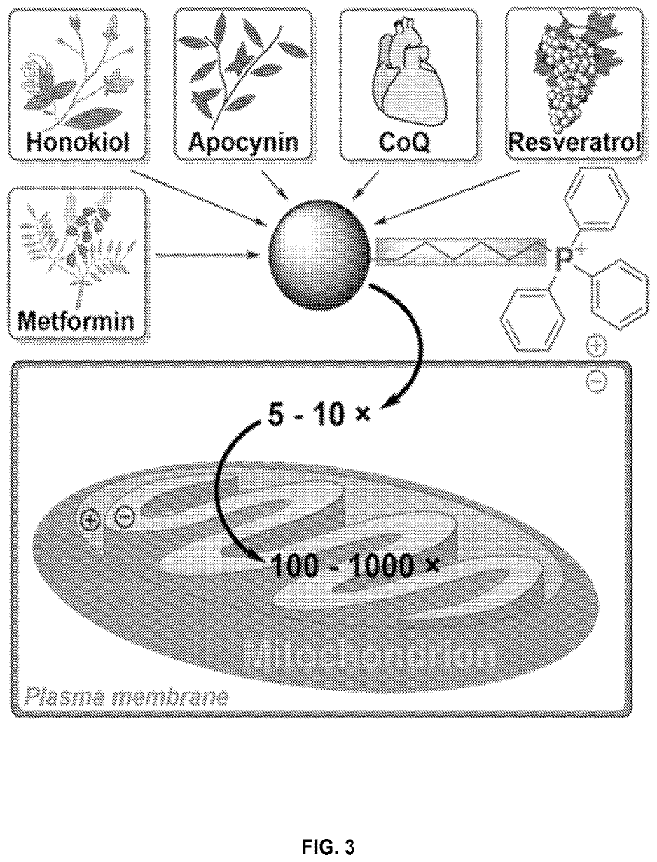

FIG. 3 depicts mitochondrial localization (amine to hydrophobicity ratio).

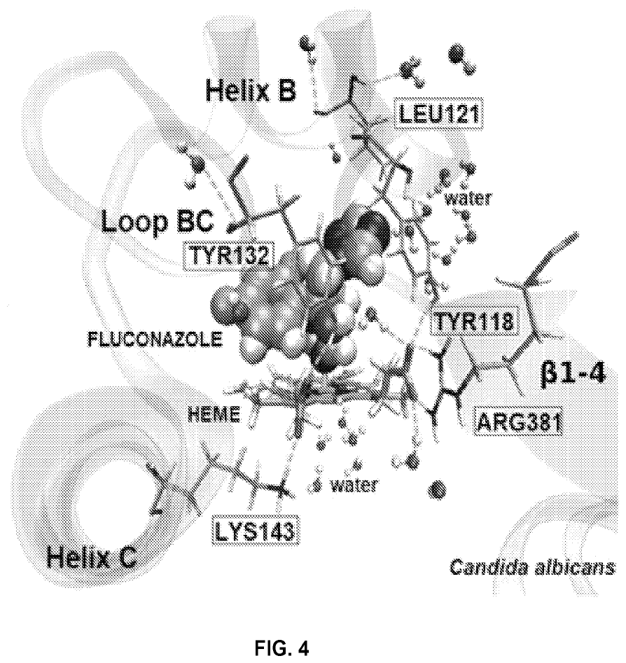

FIG. 4 depicts a structure of fluconazole bound to CYP51 heme-dependent cytochrome P450 sterol demethylase.

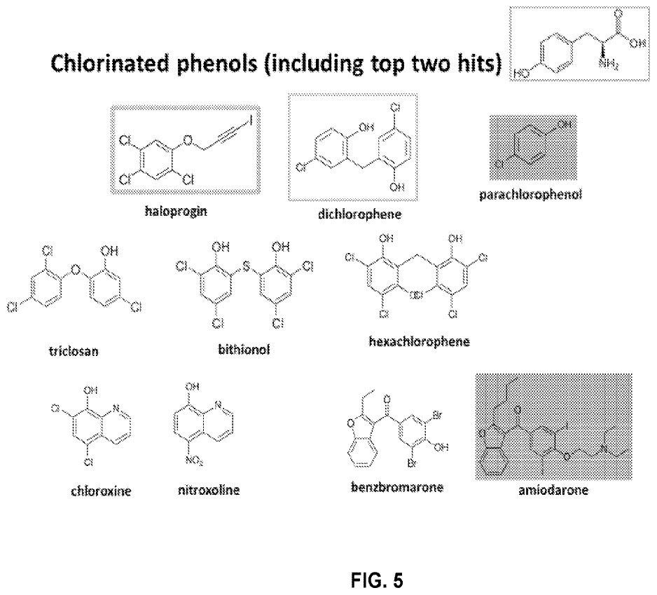

FIG. 5 depicts structures of chlorinated phenols comprising haloprogin, dichlorophene, parachlorophenol, triclosan, bithinol, hexachlorophene, chloroxine, nitroxoline, benzbromarone, and amiodarone.

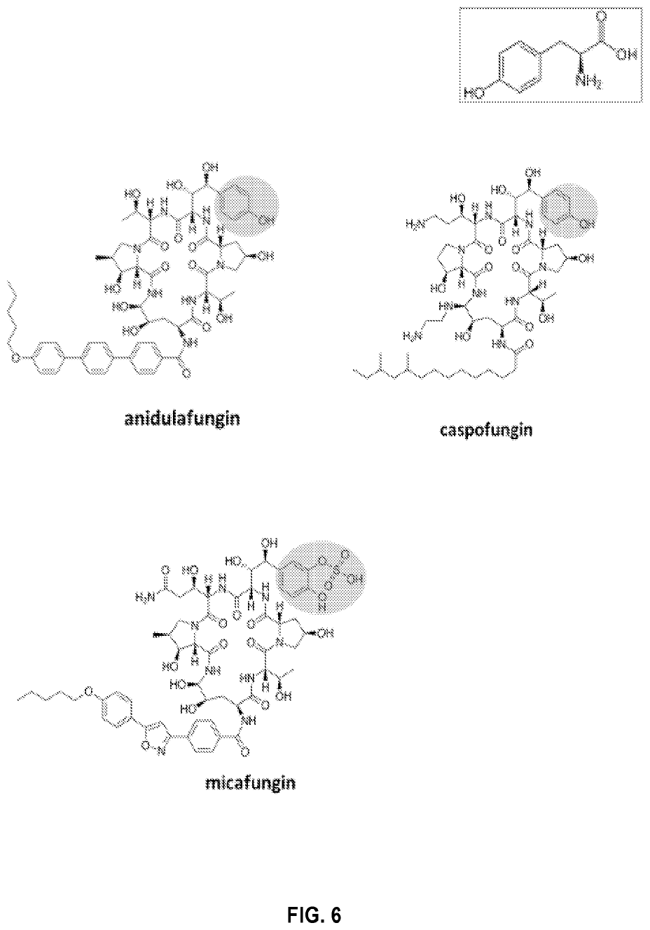

FIG. 6 depicts structures of echinocandins comprising anidulafungin, caspofungin, and micafungin.

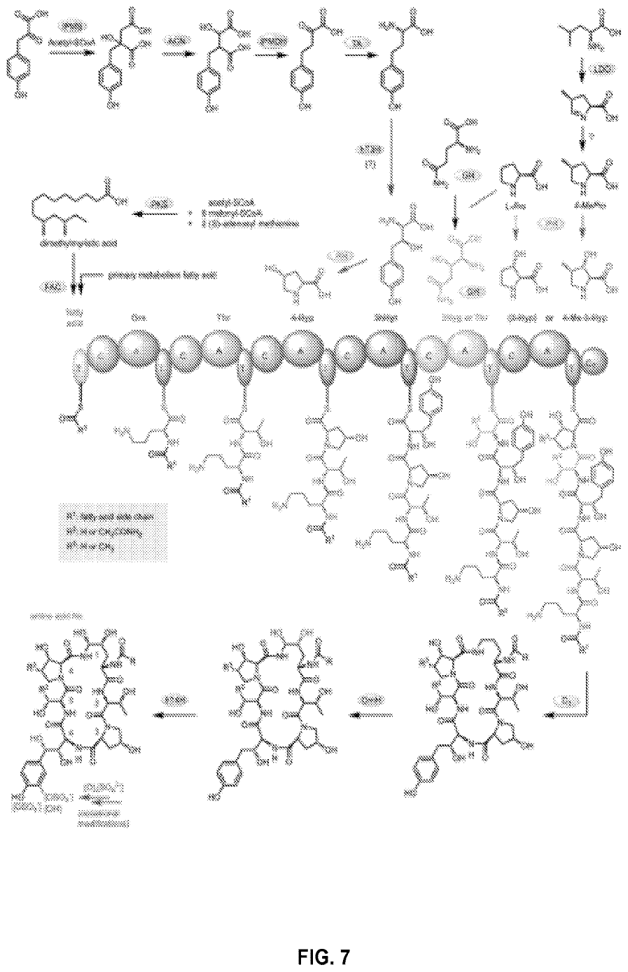

FIG. 7 depicts a structural overview of echinocandin biosynthesis (Hüttel, Z. Naturforsch, 2017; 72(1-2)c:1-20).

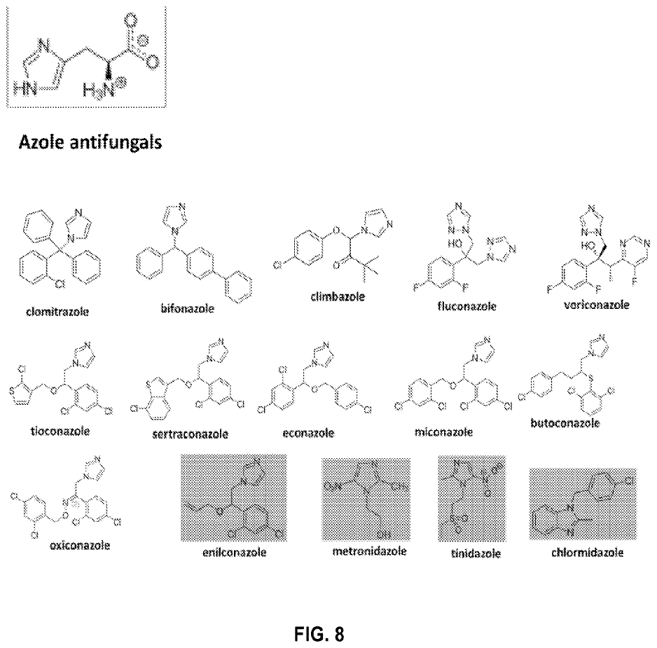

FIG. 8 depicts structures of azole antifungals comprising clotrimazole, bifonazole, climbazole, fluconazole, voriconazole, tioconazole, sertaconazole, econazole, miconazole, butoconazole, oxiconazole, enilconazole, metronidazole, tinidazole, and chlormidazole.

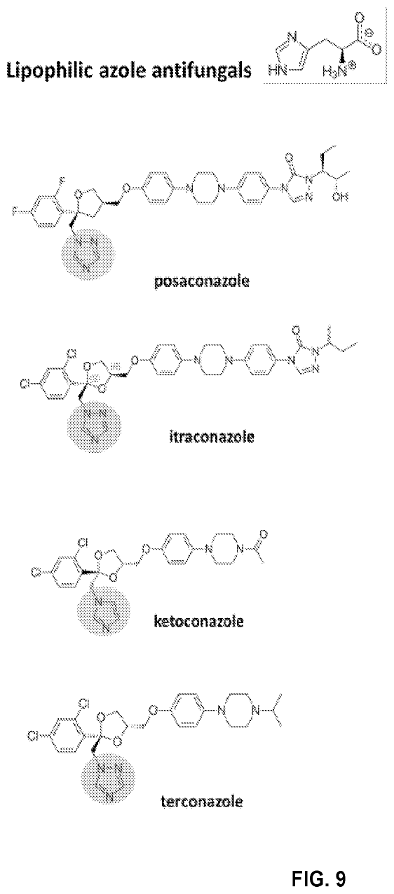

FIG. 9 depicts structures of lipophilic azole antifungals comprising posaconazole, itraconazole, ketoconazole, and terconazole.

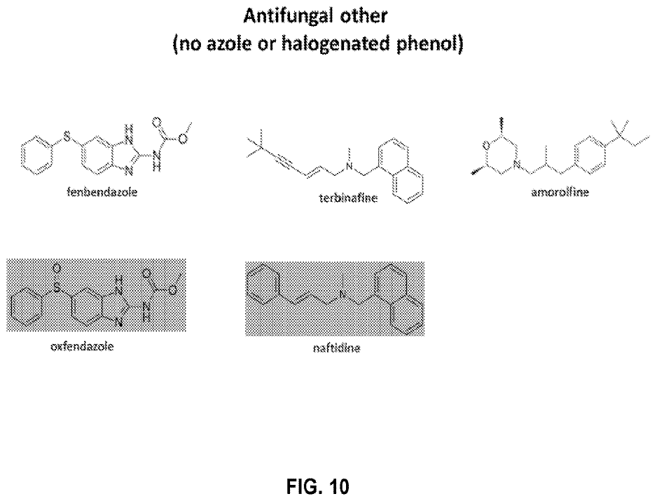

FIG. 10 depicts structures of antifungals comprising fenbendazole, terbinafine, amorolfine, oxfendazole, and naftidine. Of note, there is a very steep structure-activity relationship.

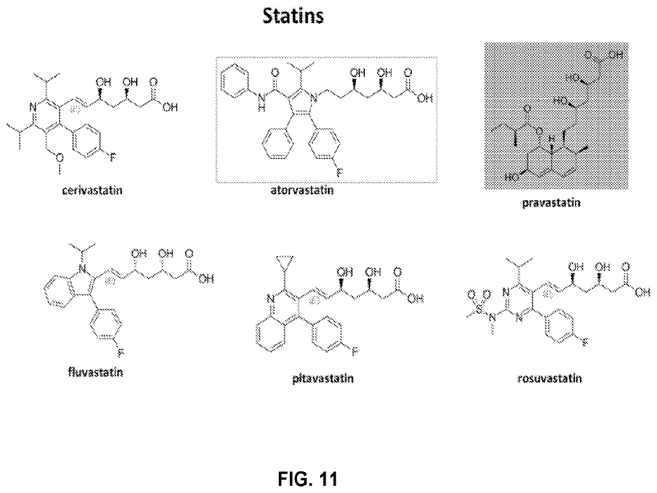

FIG. 11 depicts structures of statins comprising cerivastin, atorvastatin, pravastatin, fluvastatin, pitastatin, and rosuvastatin.

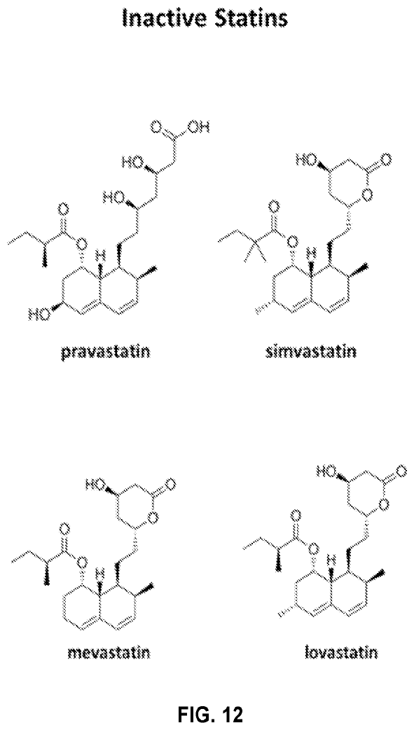

FIG. 12 depicts structures of inactive statins comprising pravastatin, simvastatin, mevastatin, and lovastatin.

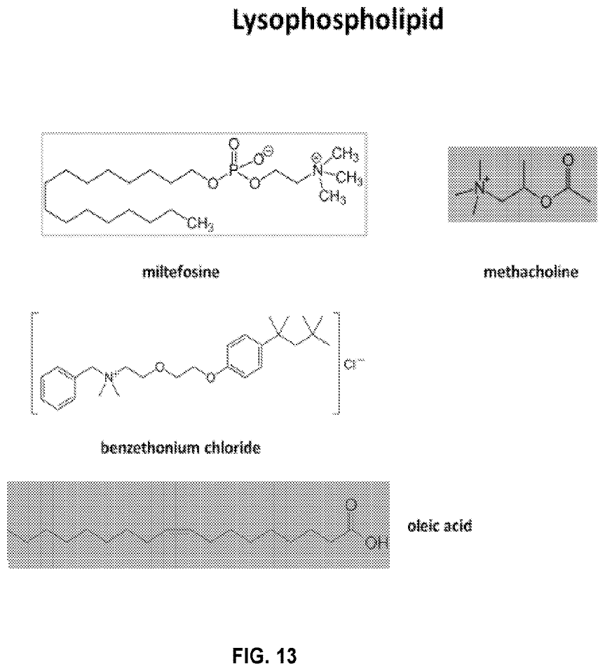

FIG. 13 depicts a structures of lysophospholipid comprising miltefosine, methacholine, benzethonium chloride, and oleic acid.

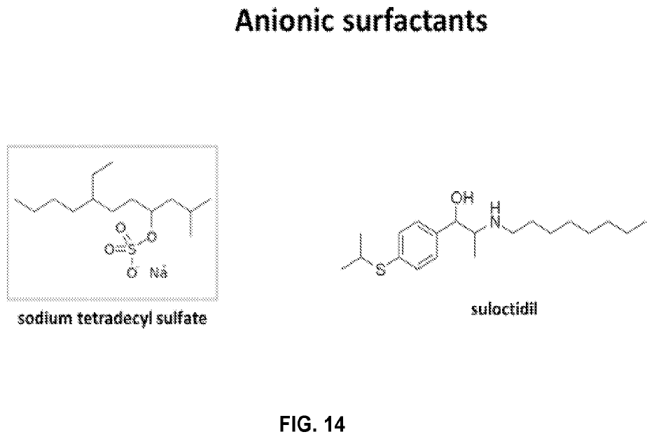

FIG. 14 depicts structures of anionic surfactants comprising sodium tetrdecyl sulfate and suloctidil.

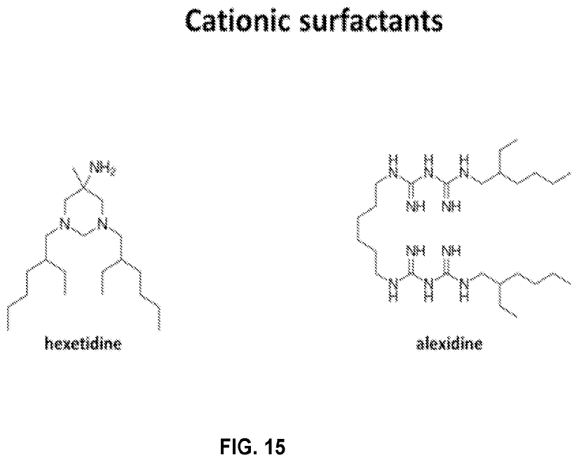

FIG. 15 depicts structures of cationic surfactants comprising hexetidine and alexidine.

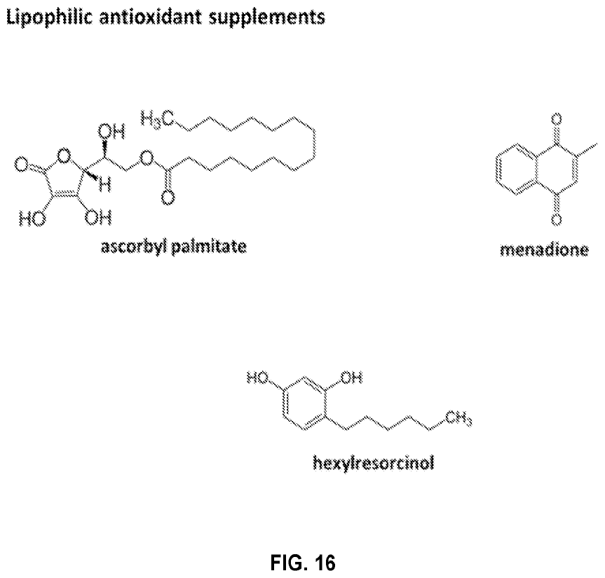

FIG. 16 depicts structures of lipophilic antioxidant supplements comprising ascorbyl palmitate, menadione, and hexylresorcinol.

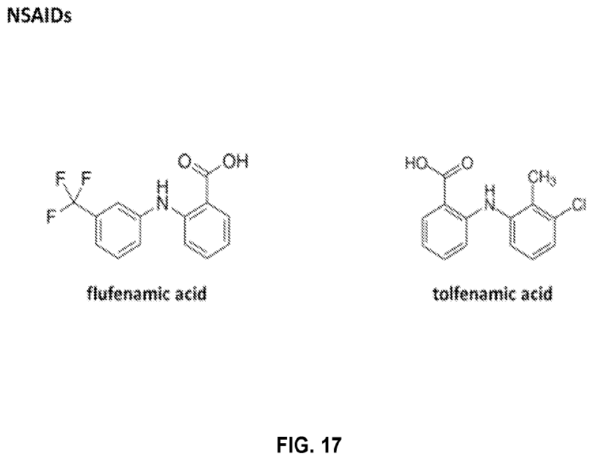

FIG. 17 depicts structures of non-steroidal anti-inflammatory drugs (NSAIDS) comprising flufenamic acid, and tolfenamic acid.

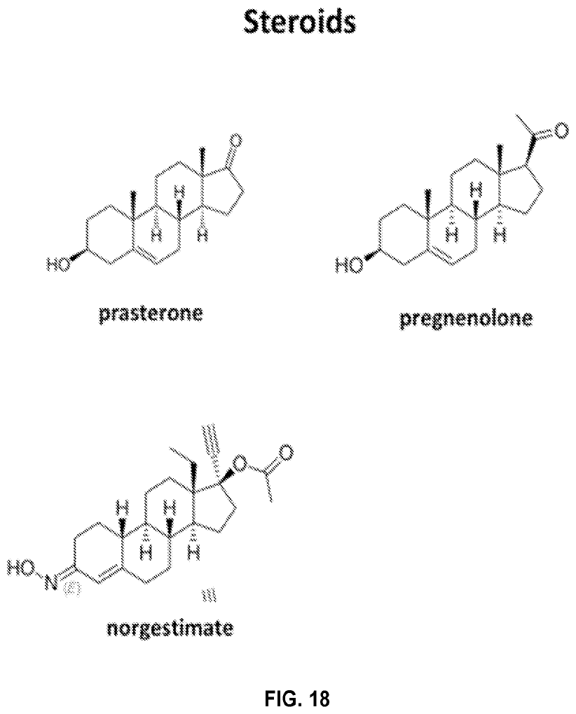

FIG. 18 depicts structures of steroids comprising prasterone, pregnenolone, and norgestimate.

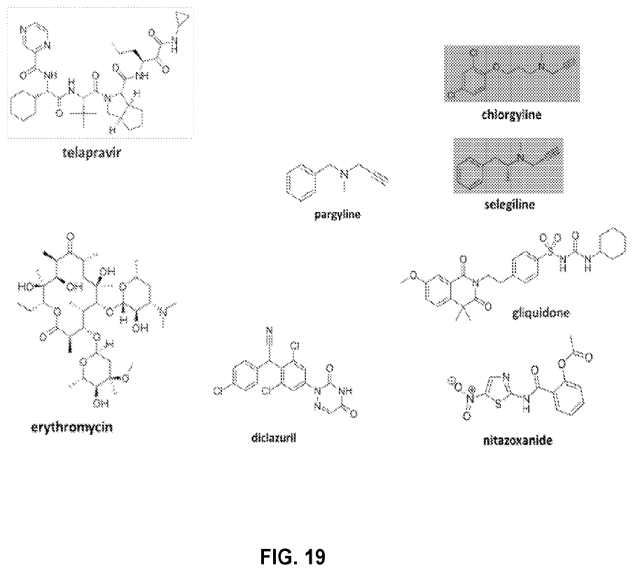

FIG. 19 depicts other compounds comprising telapravir, pargyline, chlorgyline, selegiline, erythromycin, diclazuril, gliquidone, and nitazoxanide.

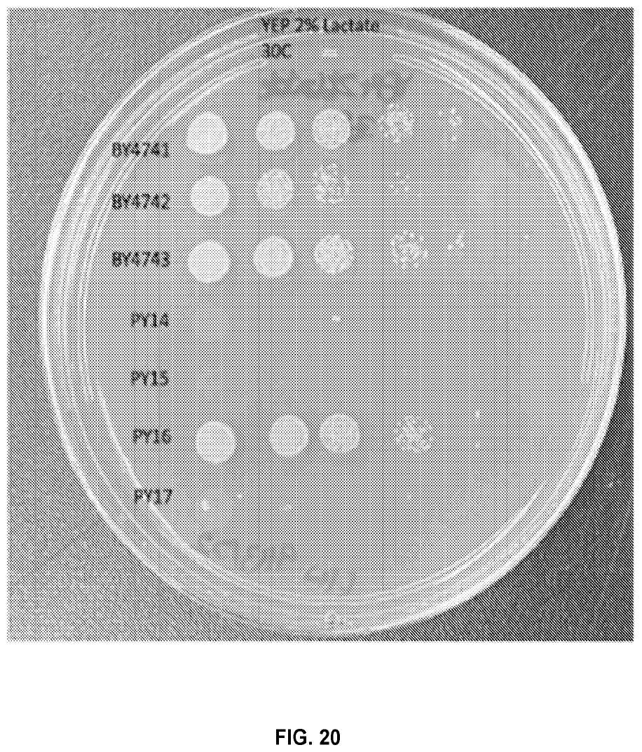

A unique hit signature was identified using the pilot screen of ~2,500 known drugs. A drug repurposing screen was performed. BY4741 and BY4742 are wildtype haploid controls, BY4743 is the wildtype diploid control. PY14 and PY15 are haploid shy1Δ knockout mutants of each yeast mating type. PY16 is the diploid SHY1/shy1Δ heterozygous mutant, which grows just like BY4743. PY17 is the diploid homozygous shy1Δ knockout mutant. FIG. 20.

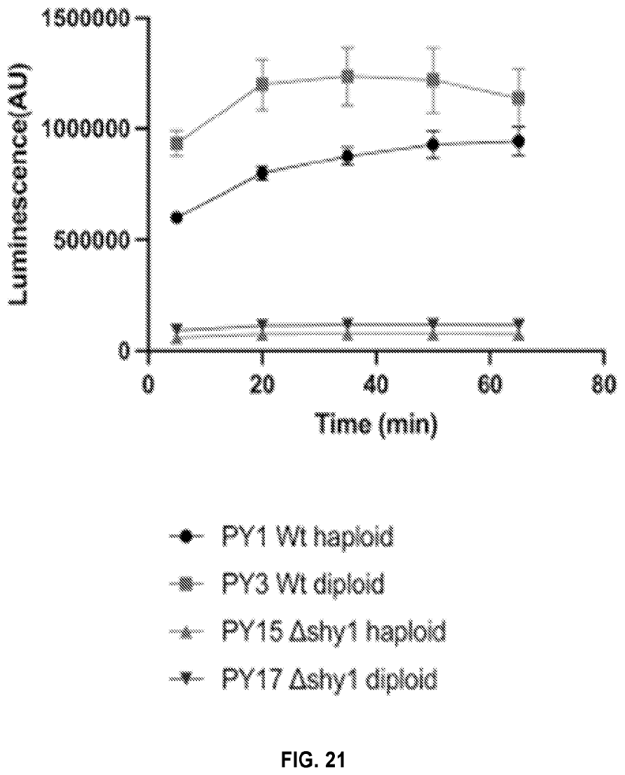

In stage two of the yeast drug repurposing workflow, a Bac-TiterGlo luminescence assay in liquid media in 96-well plates was used. The minimal growth phenotype observed on solid media was perfectly replicated in liquid media, as shown below. FIG. 21. The diploid positive control BY4743 grew faster than the haploid positive control BY4741. Similarly, the diploid shy1Δ knockout mutant grew slightly faster than the haploid shy1Δ knockout mutant.

In stage three of the modular yeast drug repurposing workflow, there was a scale up from 96-well plates to 384-well plates for the pivotal Z′ optimization experiment. This is the last step before advancing to a pilot drug repurposing screen of the Pharmakon library. The typical Z′ cutoff is 0.5, which was exceeded by both the haploid and diploid homozygous shy1Δ knockout mutants.

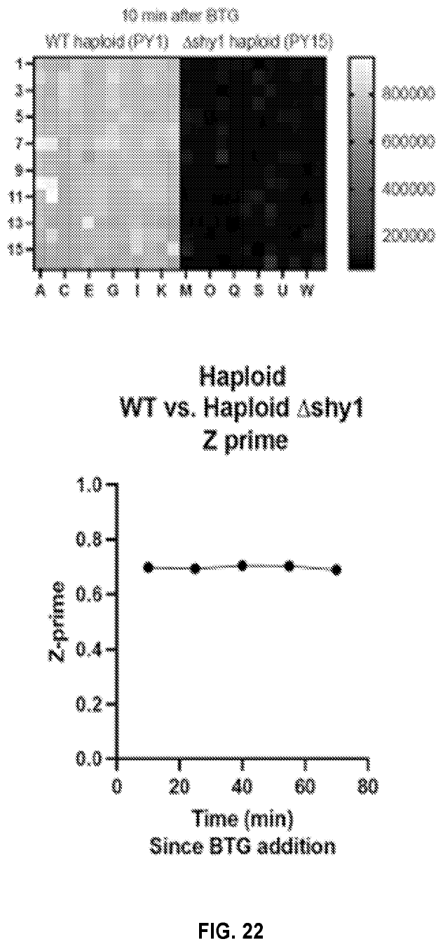

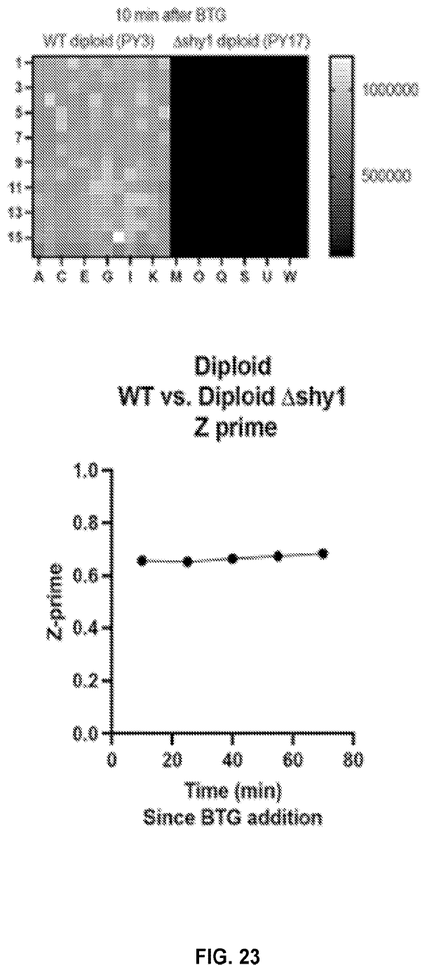

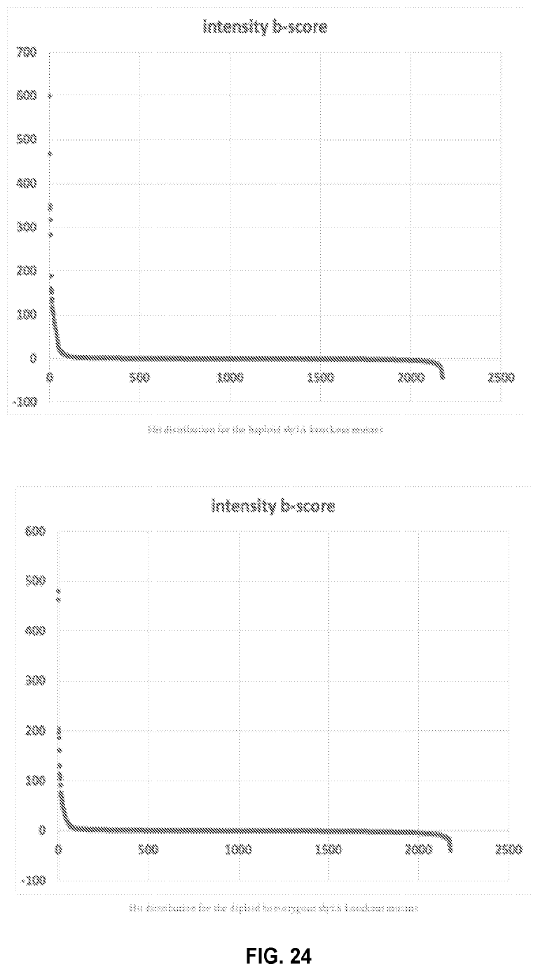

Because the Z′ was sufficiently high and because the large absolute difference in luminescence units (cell number) between the positive control and the shy1Δ knockout mutants at baseline, intensity b-scores (equivalent to a Z-score) soared into the 100s, yielding approximately 50 hits that resulted in at least a doubling of the number of shy1Δ1 knockout mutant cells compared to untreated (placebo) mutant cells. FIG. 22, FIG. 23, FIG. 24A, FIG. 24B. It is thought that the hits act by stabilizing cytochrome c oxidase.

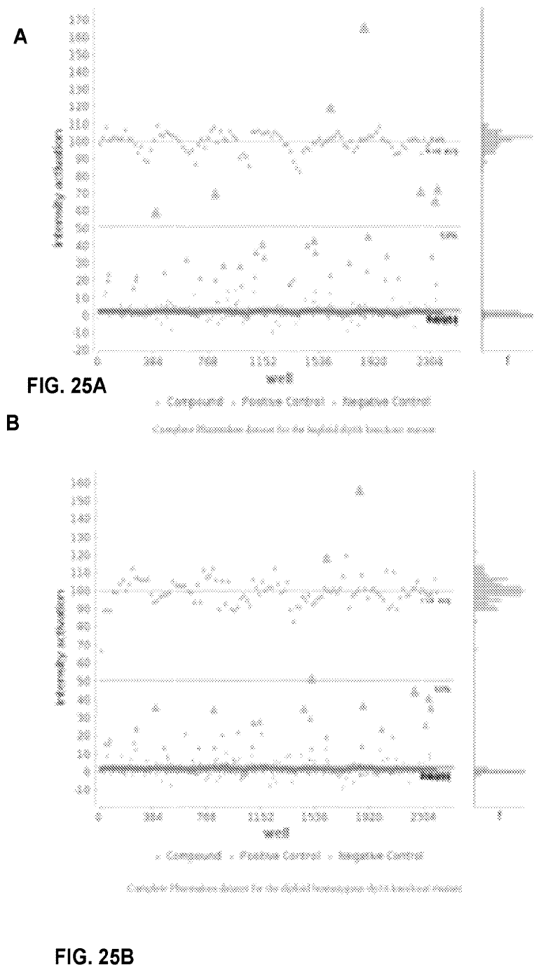

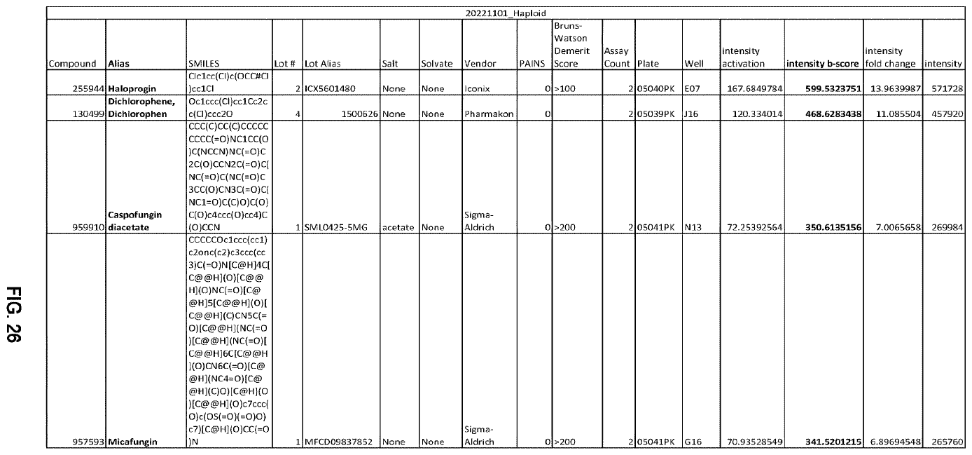

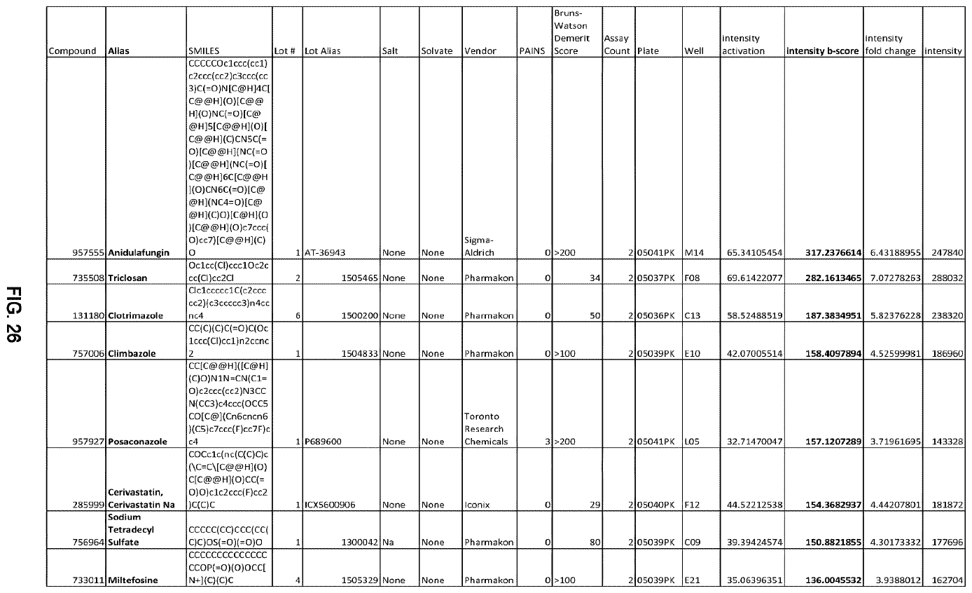

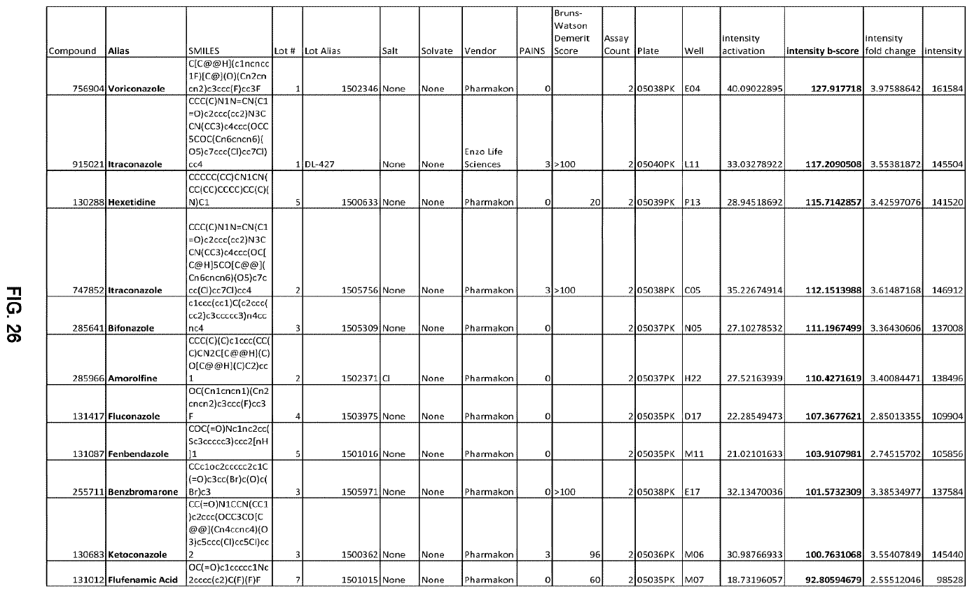

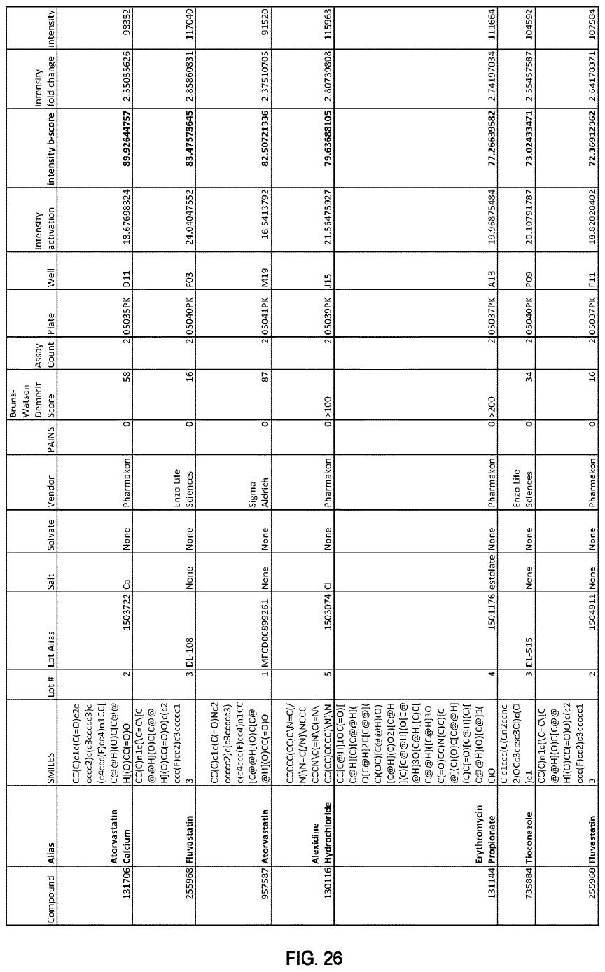

FIG. 25 depicts the full dataset showing the ~2,500 library compounds (red triangles) plotted along with the positive controls (cyan triangles) and the negative controls (green triangles) that are obscured by the baseline of inactive compounds at 0 intensity activation (cell number). The discernible red triangles are the hits; the larger the triangles, the more statistically significant the result. Two compounds resulted in growth by shy1Δ knockout mutants that exceeded or nearly exceeded the growth of the positive controls. FIG. 25A and FIG. 25B. Haploid and diploid SURF1 deficient mutants were screened. The positive control is a wildtype (unmutated) yeast strain. Highlighted columns are drug name (alias) and intensity b-score (equivalent to a Z score). Columns are sorted from highest to lowest by intensity b-score. FIG. 26 and FIG. 27 include the drug repurposing data. FIG. 26 depicts the haploid b-score rank for compounds with b-scores greater than or equal to 10 FIG. 27 depicts the diploid b-score rank for compounds with b-scores greater than or equal to 10.

SURF1-LS patients typically exhibit increased lactate in the liquor and midbrain neurodegeneration. The lack of animal models recapitulating the patient-specific neuronal pathology has hindered the discovery of treatments.

It has been demonstrated that SURF1 mutations impaired neuronal morphogenesis in induced pluripotent stem cell (iPSC)-derived neurons and cerebral organoids (Inak et al., Nat Comm 2021). The prime defects emerged at the level of neural progenitor cells, which were unable to perform the metabolic switch required for neural commitment, and thus showed inefficient neurite outgrowth. Studies here aimed to discover treatment strategies rescuing these neuronal defects using the compounds from the survival yeast screening.

In order to evaluate selected compounds' impact on mitochondrial function in patient-derived fibroblasts compared to controls, three seahorse assays were conducted, Exp. 1: Fibroblast cell line GM28010—single compounds; Exp. 2. Fibroblast cell line GM28010—combination of compounds+ Single compounds; and Exp. 3: Fibroblast cell line GM28012—single compounds. Seahorse XF24 Extracellular Flux Analyzers continuously measure oxygen concentration and proton flux in the cell supernatant over time (Wu et al., 2007). The oxygen concentration and proton flux measurements are converted in OCR and ECAR values. This enables a direct quantification of mitochondrial respiration and glycolysis. The aim of the seahorse assays is to evaluate the impact of selected compounds on mitochondrial function in patient-derived fibroblasts compared to controls. Compounds used in the seahorse assays include posacanazole, sertaconazole, glabridin, zinglibroside (ginsenoside Z R1), AM281, cysteamine bitrartate, PF-06447475, losmapimod, telepravir, miltefosine, incyclinide, poziotinib, and methylene blue.

Sertaconazole can represent an effective repositionable drug for children affected by Leigh syndrome.

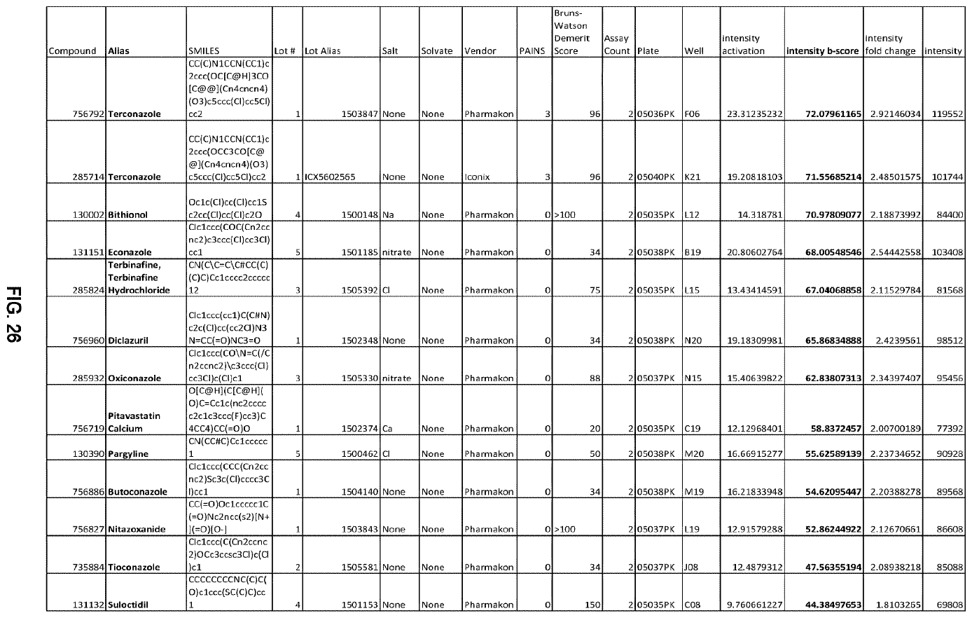

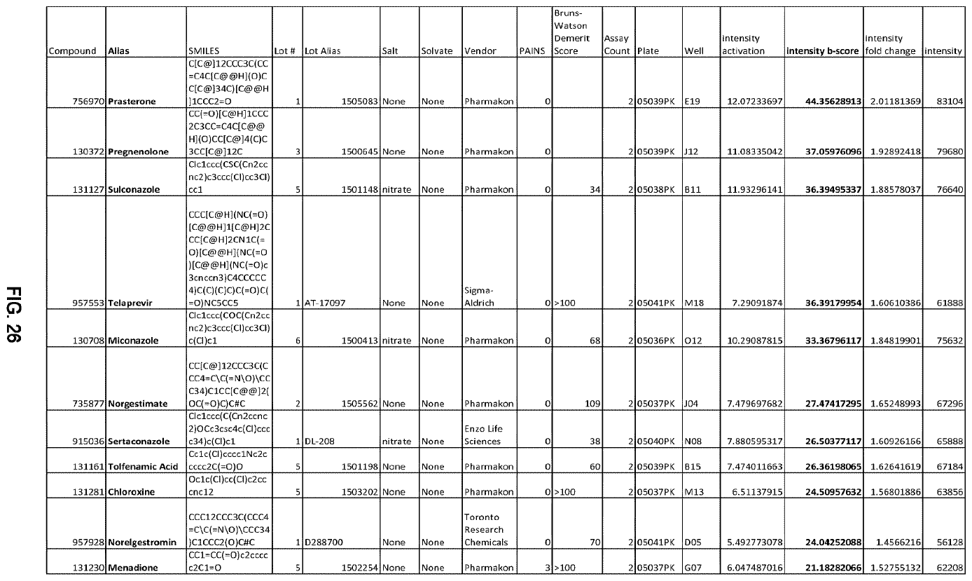

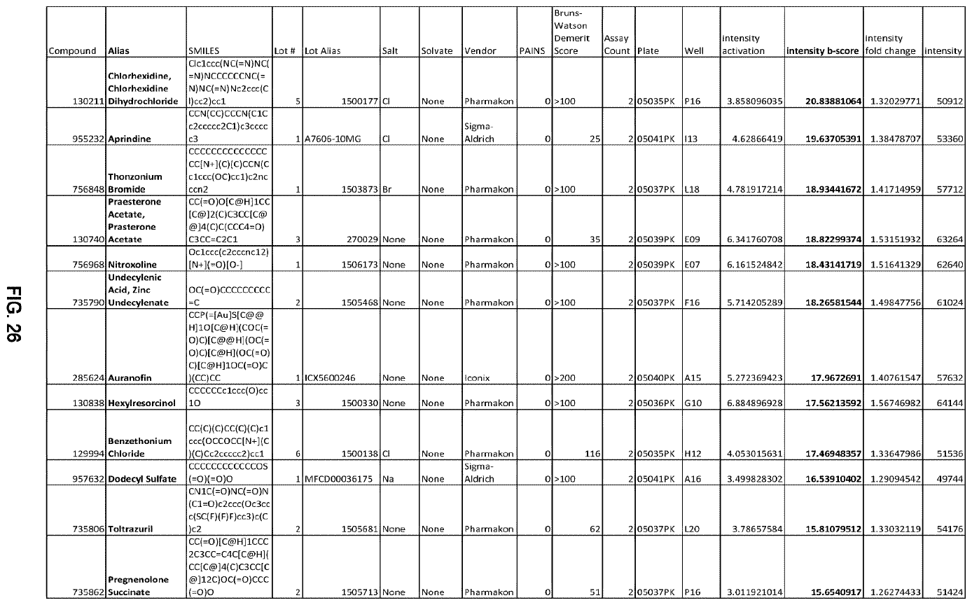

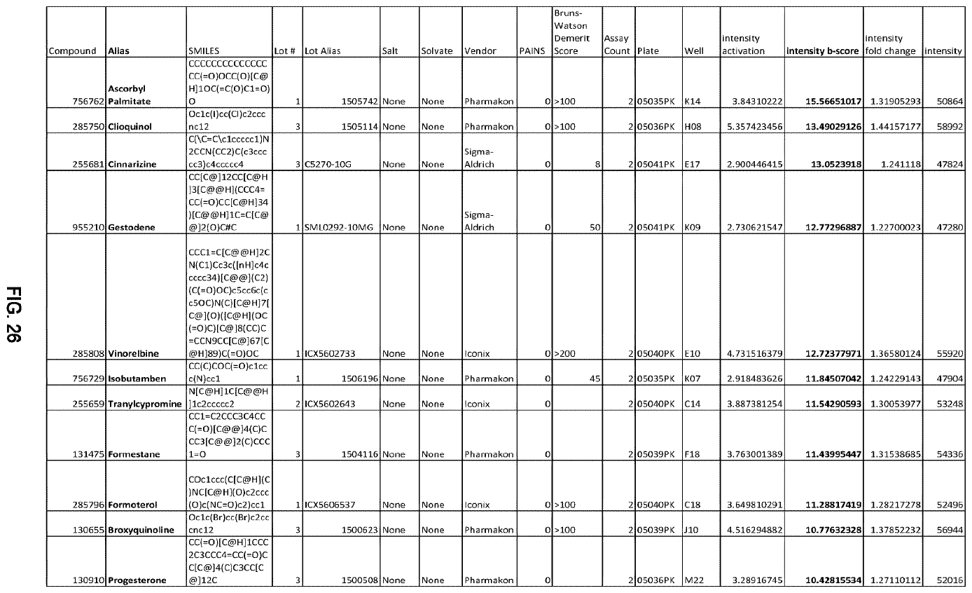

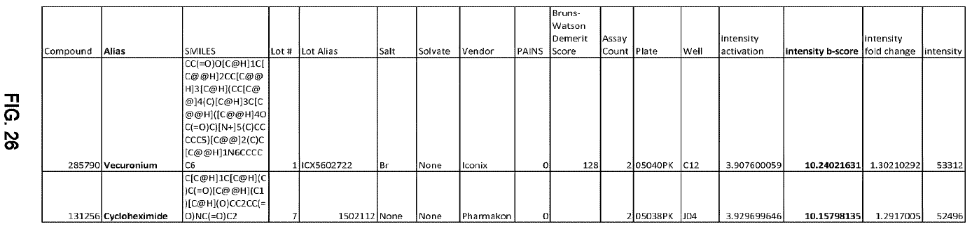

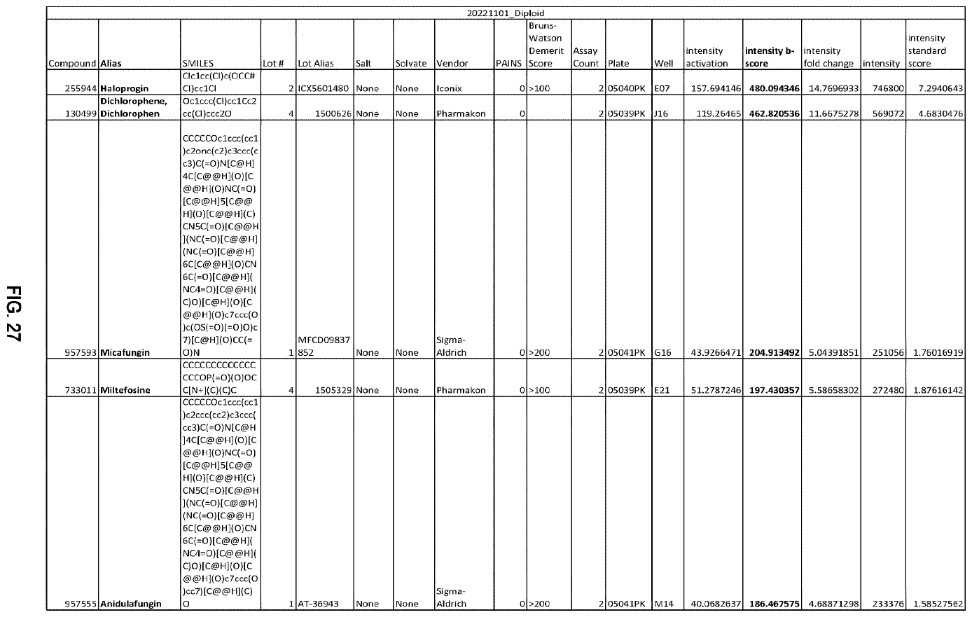

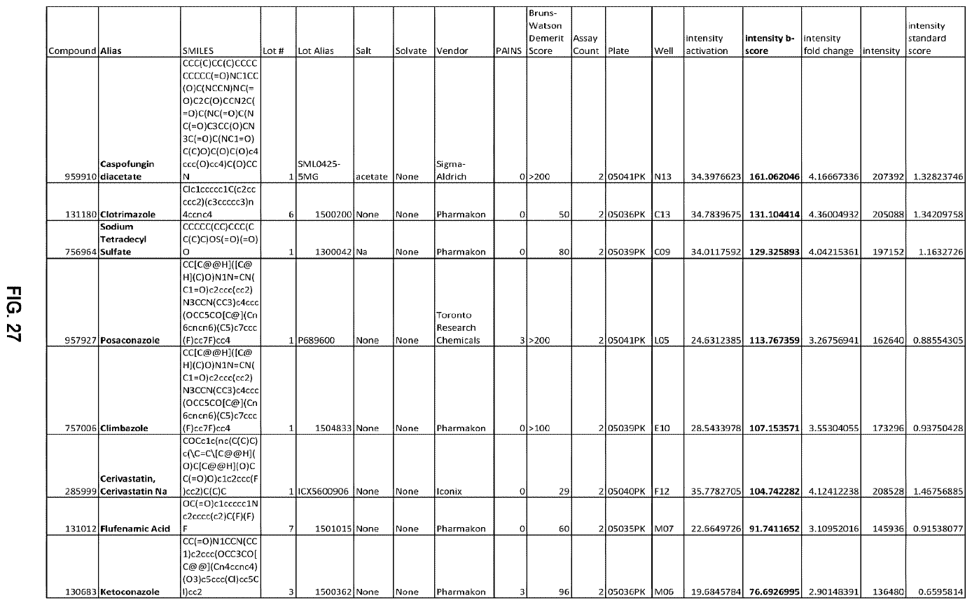

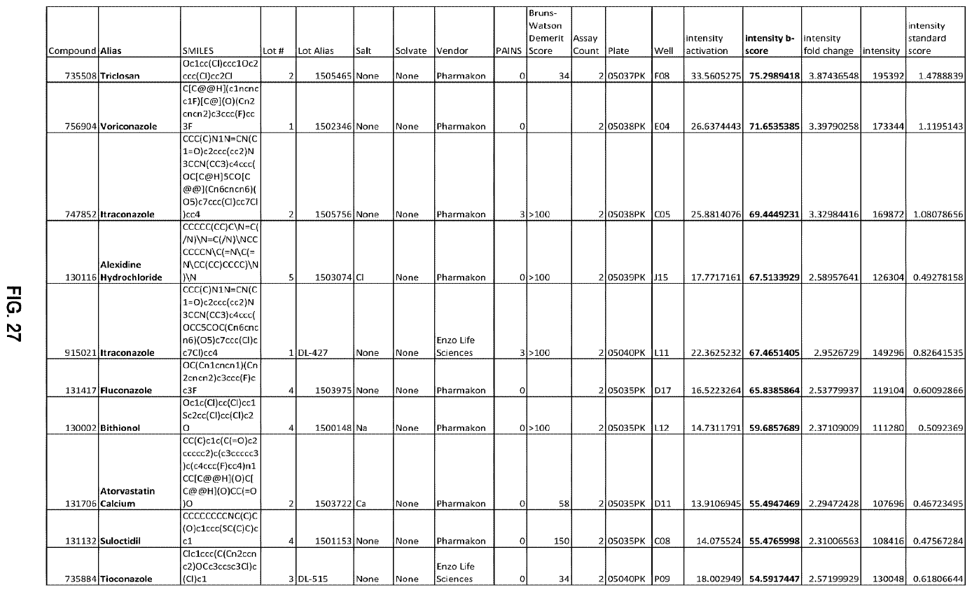

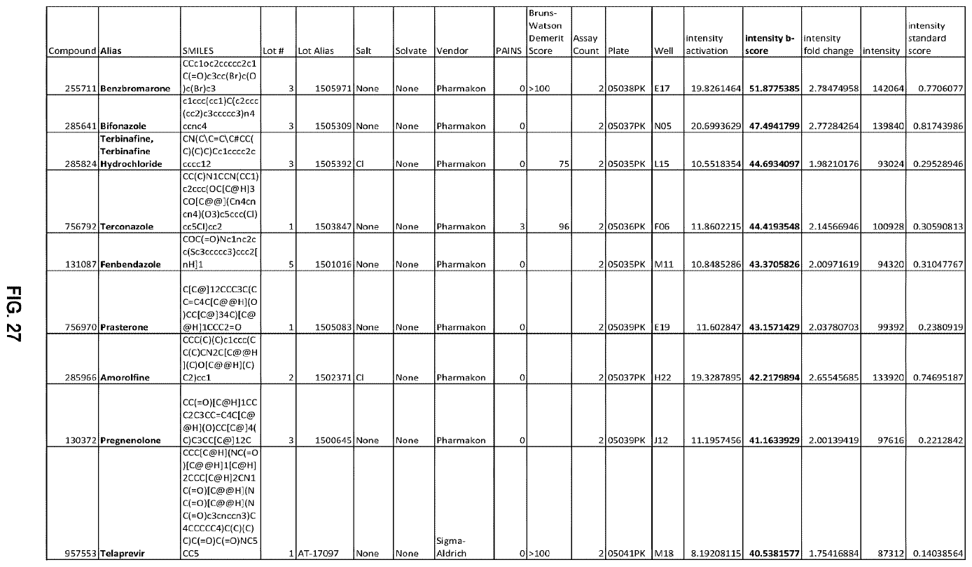

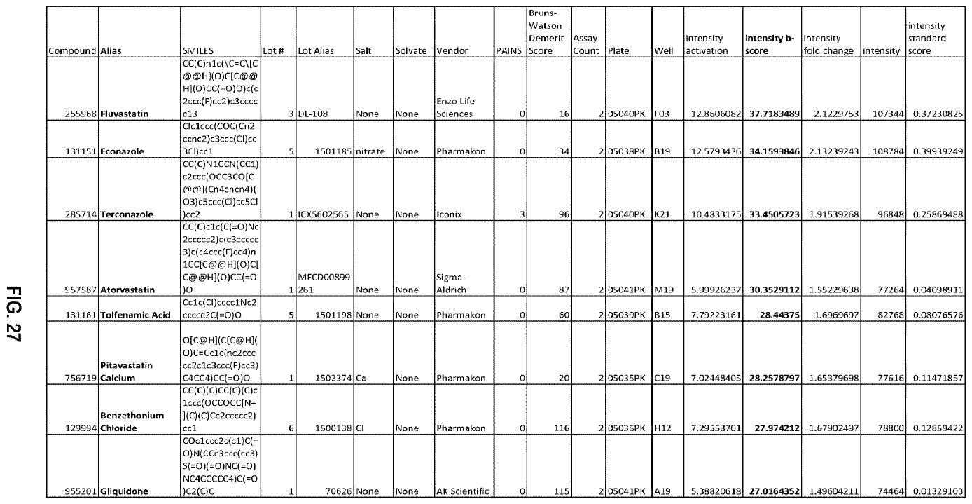

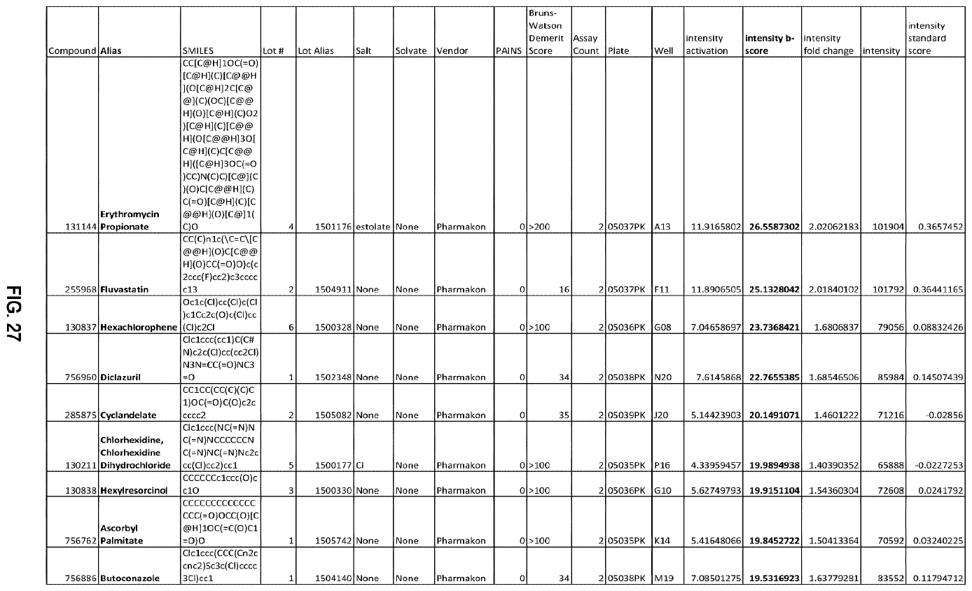

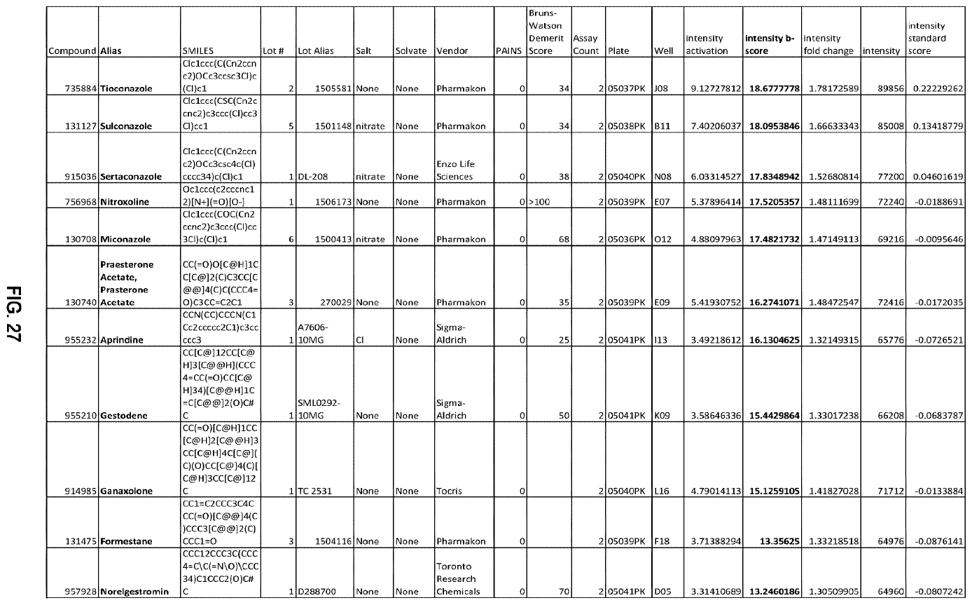

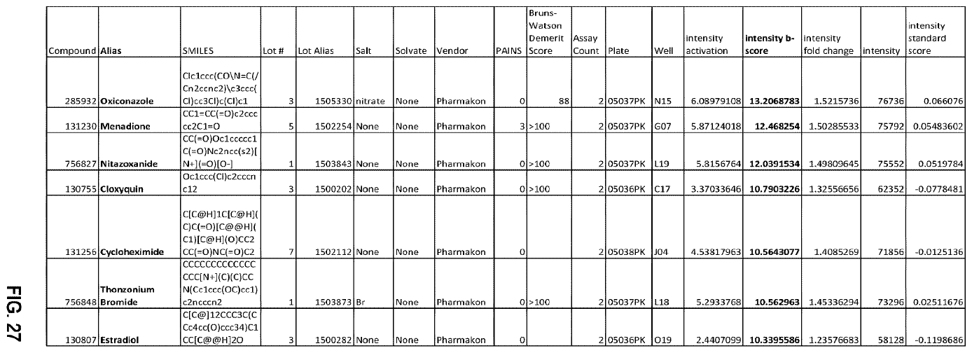

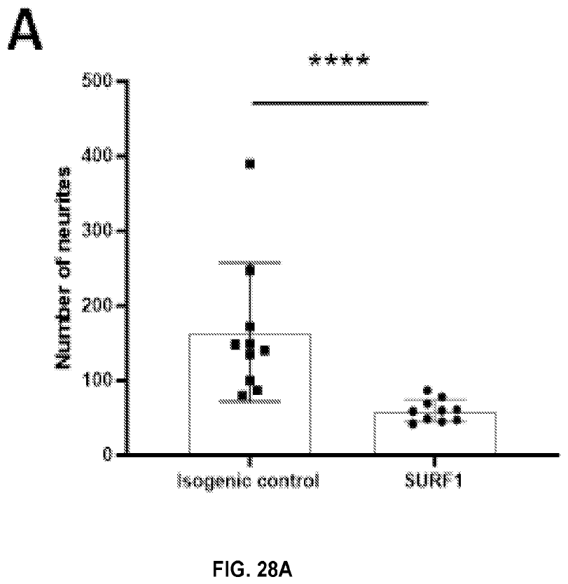

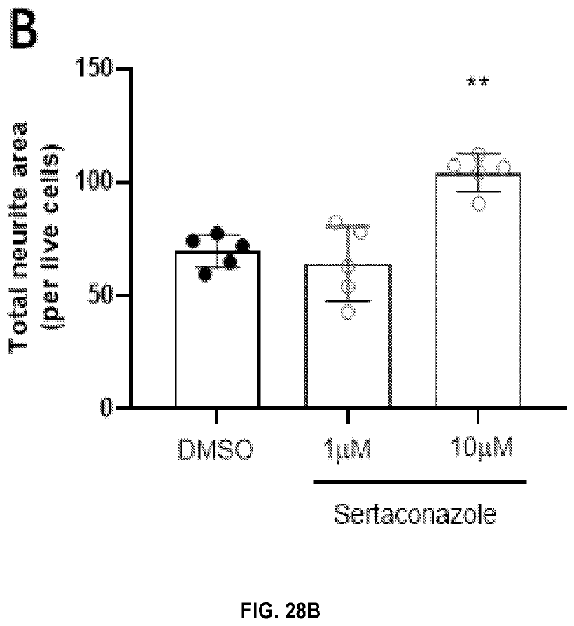

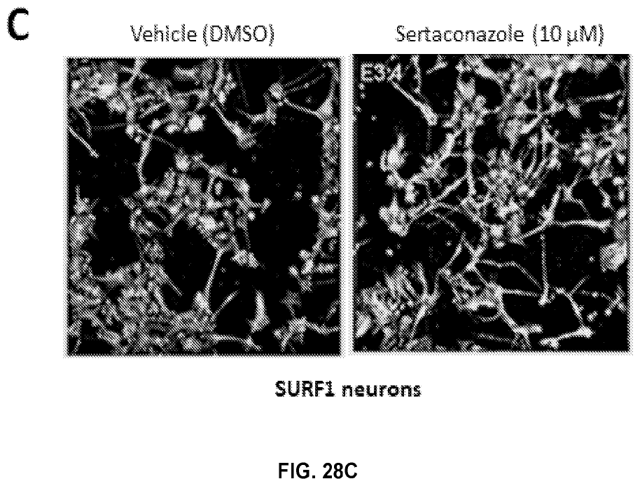

The effect of Sertaconazole in human neurons carrying mutations in the gene SURF1 was measured. Hit compounds were filtered and selected to test their effect on neuronal morphogenesis. CRISPR/Cas9 engineered isogenic SURF1 mutant iPSCs on which neural commitment via overexpression of NGN2 was forced were used. Sertaconazole was identified as being able to carry out a dose-dependent rescue of neurite outgrowth capacity (FIG. 28A, FIG. 28B, and FIG. 28C).

Sertaconazole ameliorates neuromorphogenesis defects in SURF1-mutant neurons. FIG. 28A depicts quantification of total neurite area in SURF1-mutant neurons. Each value represents the total neurite area in each well of a 96-well plate. Significant values were calculated using the non-parametric Mann-Whitney test and defective neurite development in SURF1-mutant cells was found FIG. 28B depicts quantification of total neurite area in SURF I-mutant neurons treated with either the DMSO vehicle or Sertaconazole at different concentrations. Neurite area was measured 5 days after induction of NGN2 overexpression, using high-content image analysis of MAP2-positive stained neuronal cells. Each value represents the total neurite area in each well of a 96-well plate. Correction of defective neurite development in SURF1-mutant cells was found FIG. 28C depicts representative images of SURF1 mutant neurons treated with either the DMSO vehicle or Sertaconazole.

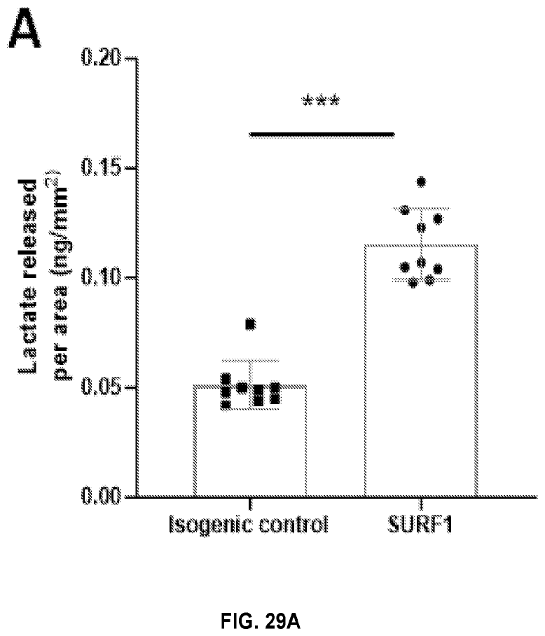

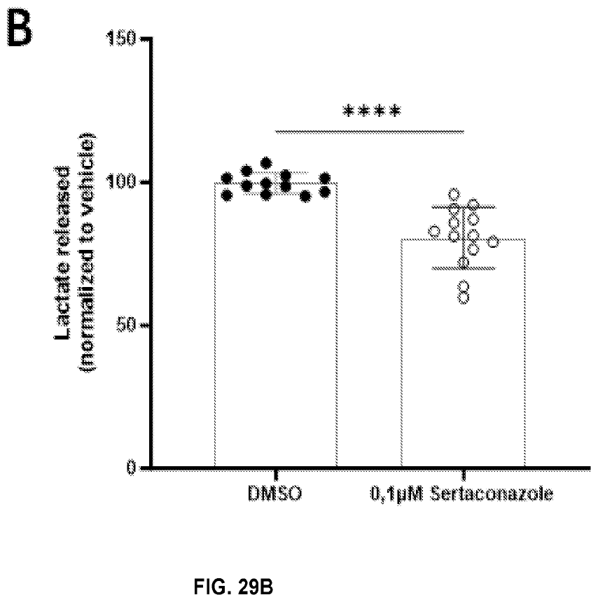

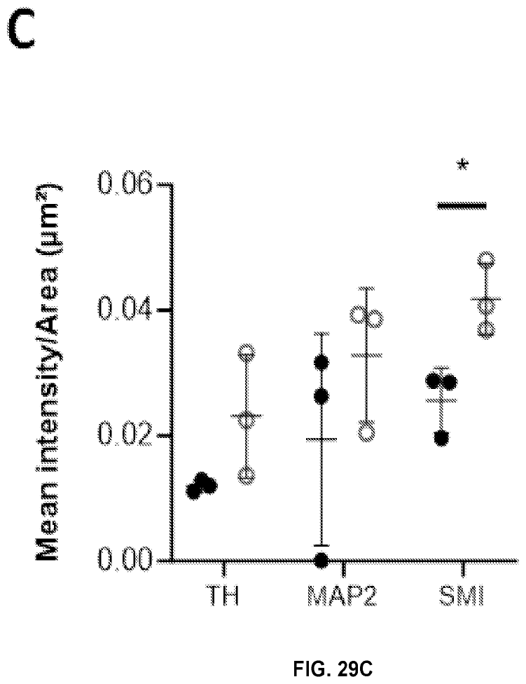

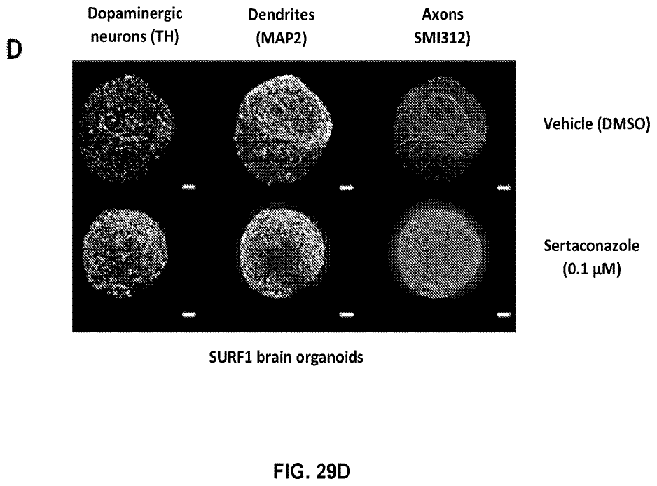

The effect of Sertaconazole in brain organoids carrying mutations in the gene SURF1 was measured. SURF1-mutant midbrain organoids showed key disease-specific features, such as increased lactate released in the media, decreased number of tyrosine hydroxylase-positive dopaminergic neurons and aberrant neuronal branching, as seen by altered dendrites (positive for MAP2) and altered axons (positive for SMI312). It was determined that Sertaconazole reduced the aberrant release of lactate in the media, increased the amount of TH-positive dopaminergic neurons, and rescued the overall neuronal branching organization (FIG. 29A, FIG. 29B, FIG. 29C, and FIG. 29D).

Sertaconazole ameliorates neuromorphogenesis and lactate defects in SURF1-mutant midbrain organoids. FIG. 29A depicts lactate released in the media by SURF1-mutant midbrain organoids compared to isogenic control organoids. There is an aberrant increase of lactate release by SURF1-mutant organoids. FIG. 29B depicts quantification of lactate in the media of SURF1-mutant midbrain organoids that were treated with either DMSO vehicle or 0.1 μM Sertaconazole for 1 week. Sertaconazole treatment decreases the aberrant amount of lactate released to the media. FIG. 29C depicts that Sertaconazole treatment corrected the increased the amount of neuronal processes in human midbrain organoids carrying a mutation in the gene SURF1 causative of Leigh syndrome. SURF1-mutant midbrain organoids were treated with either the DMSO vehicle or 0.1 μM Sertaconazole for 1 week. Sertaconazole treatment corrected the increased the amount of neuronal processes in human midbrain organoids carrying a mutation in the gene SURF1. FIG. 29D depicts representative images of SURF1-mutant midbrain organoids treated with either the DMSO vehicle or Sertaconazole.

Selected compounds' were evaluated for their impact on mitochondrial function in patient-derived fibroblasts compared to controls. Seahorse studies using fibroblast cell line GM28010 were performed. Compounds used are found in Table 1.

TABLE 1

| | Compound | Screen identified |
|---|---|---|
| 1 | Glabridin | TargetMol |
| 2 | Incyclinide | Pharmakon |
| 3 | Losmapimod | Personal communication |
| 4 | Miltefosine | TargetMol, Pharmakon, ReFrame |
| 5 | PF-06447475 | TargetMol |
| 6 | Posaconazole | TargetMol, Pharmakon, ReFrame |
| 7 | Sertaconazole | TargetMol, Pharmakon |
| 9 | Telaprevir | TargetMol |
| 10 | Poziotinib | TargetMol |
| 11 | Ginsenoside Z R1 | TargetMol |
| | (Zinglibroside) | |
| 12 | AM281 | TargetMol |

Fibroblasts provided the characteristic response to injected compounds during the Seahorse assay. Fibroblasts responded to galactose culturing for 48 hours by increasing basal respiration rates but not maximal respiration. GM28010 SURF1 mutant fibroblasts had lower respiration rates compared with GM0038F control fibroblasts in both glucose and galactose culturing conditions. Respiration was decreased by approximately 50% or more in galactose and glucose-cultured cells. SURF1 mutant cells compensated with increased glycolysis. Experimental compounds did not always have the same respiratory effects in glucose and galactose-cultured cells. The compound that showed the most interesting results on respiration was losmapimod. Increased respiration in both glucose and galactose culture conditions. Respiration was increased up to 15%. Increasing the treatment time and increasing the dose-range may be useful follow-up experiments.

A combination of compounds was also tested with fibroblast cell line GM28010. The compounds were used in this experiment are shown in Table 2.

TABLE 2

| | Compound | Screen identified |
|---|---|---|
| 1 | Losmapimod | Personal communication |
| 2 | PF-06447475 | TargetMol |
| 3 | Ginsenoside Z R1 (Zinglibroside) | TargetMol |
| 4 | Methylene Blue | Personal communication |
| 5 | cysteamine bitartrate | Personal communication |

It was determined that 1) Treatment with the double combination of losmapimod and ginsenoside slightly increased respiration and ATP production rate through OXPHOS while decreasing ATP production through glycolysis; 2) Treatment with the double combination of ginsenoside and PF-06447475 slightly increased respiration and ATP production rate through OXPHOS while maintaining total ATP production rates; and 3) Treatment with the double combination of losmapimod and PF-06447475 slightly increased respiration and ATP production rate through OXPHOS while maintaining total ATP production rates.

Single compounds were tested with fibroblast cell line GM28012. Compounds used are shown in Table 4.

TABLE 4

| | Compound | Screen identified |
|---|---|---|
| 1 | Glabridin | TargetMol |
| 2 | Incyclinide | Pharmakon |
| 3 | Losmapimod | Personal communication |
| 4 | Miltefosine | TargetMol, Pharmakon, ReFrame |
| 5 | PF-06447475 | TargetMol |
| 6 | Posaconazole | TargetMol, Pharmakon, ReFrame |
| 7 | Sertaconazole | TargetMol, Pharmakon |
| 9 | cysteamine bitartrate | Personal communication |
| 10 | Poziotinib | TargetMol |
| 11 | Ginsenoside Z R1 | TargetMol |
| | (Zinglibroside) | |
| 12 | AM281 | TargetMol |

Fibroblasts provided the characteristic response to injected compounds during the Seahorse assay. Glucose plate 2 displayed higher maximal respiration that glucose plate 1 cells. Fibroblasts responded to galactose culturing for 48 hours by increasing basal respiration rates but not maximal respiration. Experimental compounds did not always have the same respiratory effects in glucose and galactose cultured cells or comparing to GM28010, another SURF1 cell line previously tested. None of the compounds tested increased maximal respiration. The compounds that showed the most interested results on respiration and total ATP production were:

### Losmapimod

Increased ATP-linked respiration and ATP production through OXPHOS and without changes in the total ATP production rate
Effect of losmapimod was not as impressive as was observed in GM28010

### Telaprevir

Dose-dependent increase in ATP production through OXPHOS and without changes in the total ATP production rate

### Cysteamine Bitartrate

Dose-dependent increase in ATP production through OXPHOS and total ATP production rate.

### Ginsenoside Z R1

1 μM concentration increased ATP production through OXPHOS and total ATP production rate.

Comparison of the results of the seahorse assay in the GM28010 and GM28012 cells is shown in Table 5.

TABLE 5

| Compound | Will's Fibroblast GM28010 | Delta-like Fibroblast GM28012 |
|---|---|---|
| Posacanazole | ↓ respiration; toxic at all conc | ↓ respiration, total ATP rate |
| | | compensated by glycolysis |
| | | until highest concentration |
| | | (10 uM) where ATP drops |
| Sertaconazole | ↓ maximal OCR@ all conc; | Maximal OCR maintained at |
| | no effect on ATP production | lower concentration; ↑ATP |
| | | via glycolysis at higher conc. |
| Glabridin | No effect on respiration in | ↓ maximal OCR in both |
| | both conditions | conditions |
| Zinglibroside | No effect on respiration in | No effect on respiration in |
| | both conditions: ↑ATP via | both conditions; ↑ATP via |
| | glycolysis and OXPHOS @ | glycolysis and OXPHOS @ |
| | 10 μM | all conc |
| AM281 | No effect on respiration in | dose-dependent ↓ maximal |
| | both conditions; ↑ATP via | OCR. ↑ATP via glycolysis @ |
| | glycolysis @5 and 10 μM | 1 μM and 5 μM |
| Cysteamine | No effect on respiration in | ↑basal respiration and ATP- |
| Bitrartate | both conditions; ↓ATP via | Linked respiration in a dose- |
| | glycolysis @ 10 μM | dependent manner in both |
| | | conditions. ↓ATP production |
| | | through glycolysis |
| PF-06447475 | No effect on respiration in | No effect on respiration in |
| | both conditions. ↑ ATP | both conditions. ↑ ATP |
| | production through both | production through |
| | OXPHOS and glycolysis. | glycolysis. At 1 μM |
| Losmapimod | 12-15% increases in | ↑ATP-linked respiration in |
| | respiration in ATP-linked and | glucose-cultured cells. ↓ATP |
| | maximal respiration in both | production via glycolysis, |
| | conditions. ↓ ATP production | total ATP production rate |
| | through glycolysis. | maintained. % ↑lower than vs |
| | | Will's fibroblasts. |
| Telepravir | No effect on respiration in | No effect on respiration in |
| | both conditions | galactose. Glucose: dose- |
| | | dependent ↑ATP-Linked |
| | | respiration at ATP production |
| | | through OXPHOS, no change |
| | | in maximal respiration. |
| Miltefosine | No effect on respiration in | No effect on respiration in |
| | both conditions | both conditions; ↑ in ATP |
| | | production through |
| | | glycolysis. |
| Incyclinide | ↑ basal respiration and ATP- | Toxicity observed in glucose |
| | linked respiration in | media |
| | galactose-cultured cells. | |
| | Toxicity in glucose | |
| Poziotinib | No effect on respiration in | Not tested |
| | both conditions; ↑ATP via | |
| | glycolysis and toxicity at | |
| | higher concentration | |

In an embodiment, a compound listed herein can be administered without another compound listed herein In an embodiment, a compound listed herein can be administered with another compound listed herein.

Provided herein are pharmaceutical compositions. In any of the embodiments, the pharmaceutical composition comprises one or more of the compounds listed herein.

In any of the embodiments, the pharmaceutical composition also comprises one or more pharmaceutically acceptable excipient(s). In some of any embodiments, the one or more excipient(s) comprises a pharmaceutically acceptable liquid carrier. In some of any embodiments, the one or more excipient(s) comprises a pharmaceutically acceptable processing agents. In some of any embodiments, the pharmaceutical composition is a liquid formulation, a formulation for an intravenous injection, a solid dosage form, or an inhalable preparation.

Also provided herein are any of the provided pharmaceutical compositions for treating a disease or disorder. In any of the embodiments, the pharmaceutical composition is to be administered to a subject having the disease or disorder.

Also provided herein are methods of treatment. In any of the embodiments, the methods involve administering one or more of the compounds provided herein or any of the pharmaceutical compositions provided herein to a subject having the disease or disorder.

Also provided herein are uses of one or more of the compounds provided herein or any of the pharmaceutical compositions provide herein in the manufacture of a medicament for the treatment of a disease or disorder.

Also provided herein are uses of one or more of the compounds provided herein or any of the pharmaceutical compositions provide herein for the treatment of a disease or disorder.

In any of the embodiments, the method or the use is a therapeutic use or a prophylactic use. In any of the embodiments, the therapeutic use is for induction therapy. In some of any of the provided embodiments, the induction therapy spans for up to at or about one week, up to at or about two weeks, up to at or about three weeks, or up to at or about four weeks. In any of the provided embodiments, the therapeutic use is for maintenance therapy. In any of the provided embodiments, the maintenance therapy spans for up to at or about one week, up to at or about two weeks, up to at or about three weeks, or up to at or about four weeks.

In some of any of the provided embodiments, the administration is selected from among intravenous, oral, parenteral, sublingual, by inhalation, rectal or topical. In some of any of the provided embodiments, the administration is intravenous administration.

In any of the embodiments, compositions comprising one or more of the compounds provided herein may be in any form suitable for the intended method of administration. In some embodiments, the compositions, such as pharmaceutical compositions, can be in any form suitable for the intended method of administration. In some embodiments, the compositions, such as pharmaceutical compositions comprising the provided one or more compounds, are formulated for administration by inhalation, parenteral administration, sublingual administration, rectal administration, or topical administration. In some embodiments, topical administration may also involve the use of transdermal administration, such as transdermal patches or iontophoretic devices. In some embodiments, the compositions, such as pharmaceutical compositions such as pharmaceutical compositions comprising the provided one or more compounds, are formulated to provide unit dosage forms, such as single or multiple unit dosage forms.

### Liquid Dosage Forms

In some embodiments, a provided composition, such as a pharmaceutical composition comprising one or more of the compounds provided herein, can be in the form of a solution, a suspension, or an emulsion. In some embodiments, non-limiting examples of liquid carriers include water, saline, pharmaceutically acceptable organic solvent(s), pharmaceutically acceptable oils and fats as well as mixtures of any two or more thereof. In some embodiments, the liquid carrier comprises other suitable pharmaceutically acceptable excipients such as solubilizers, emulsifiers, nutrients, buffers, preservatives, suspending agents, thickening agents, viscosity regulators, stabilizers. Non-limiting examples of suitable organic solvents include monohydric alcohols, such as ethanol, and polyhydric alcohols, such as glycols. Non-limiting examples of suitable oils include, soybean oil, coconut oil, olive oil, safflower oil, cottonseed oil. In some embodiments, particularly when parenteral administration is contemplated, the carrier can be an oily ester such as ethyl oleate, isopropyl myristate. Parenteral administration includes subcutaneous injections, intravenous administration, intramuscular administration, intrasternal injections, transdermal or transmucosal administration, or infusion techniques. In some aspects, any of the provided pharmaceutical compositions, such as pharmaceutical compositions comprising one or more of the compounds provided herein, are formulated for intravenous administration.

Parenteral administration includes subcutaneous injections, intravenous administration, intramuscular administration, intrasternal injections, transdermal or transmucosal administration, or infusion techniques.

In some embodiments, the provided compositions, such as pharmaceutical compositions comprising one or more of the compounds provided herein, are in the form of microparticles, microcapsules, liposomal encapsulates as well as combinations of any two or more thereof. In some embodiments, the provided compositions, such as pharmaceutical compositions comprising the one or more of the compounds provided herein, comprise liposomes. In some aspects, liposomes are generally derived from phospholipids or other lipid substances. In some embodiments, liposomes are formed by mono- or multilamellar hydrated liquid crystals that are dispersed in an aqueous medium. In some embodiments, any non-toxic, physiologically acceptable and metabolizable lipid capable of forming liposomes can be used in formulation with one or more of the compounds provided herein. In some embodiments, the provided compositions, such as pharmaceutical compositions comprising lipids or liposomes, further comprise stabilizers, preservatives, excipients. Typical lipids are the phospholipids and phosphatidyl cholines (lecithins), both natural and synthetic. Methods of forming liposomes are known in the art and are described in Prescott, Ed., “Methods in Cell Biology”, Volume XIV, Academic Press, New York, N.W., p. 33 et seq. (1976), which is incorporated herein by reference.

Injectable preparations (such as, for example, sterile injectable aqueous or oleaginous suspensions) may be formulated using exemplary methods and materials, such as, for example, suitable dispersing, wetting, and suspension agents. The sterile injectable preparation may also be a solvent, for example, as a solution in 1,3-propanediol. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed including synthetic mono- or diglycerides. In addition, fatty acids such as oleic acid find use in the preparation of injectables.

### Solid Dosage Forms

Solid dosage forms for oral administration may include capsules, tablets, pills, powders, and granules. In such solid dosage forms, the active compound may be admixed with at least one inert diluent such as sucrose, lactose, or starch. Such dosage forms may also comprise additional substances other than inert diluents, such as, for example, lubricating agents (e.g., magnesium stearate). In the case of capsules, tablets, and pills, the dosage forms may also comprise buffering agents. Tablets and pills can additionally be prepared with enteric coatings.

### Carriers, Vehicles, and Excipients

In some embodiments, the provided compositions, such as pharmaceutical compositions, further comprise a carrier or an excipient, such as a pharmaceutically acceptable carrier (or vehicle) or excipient. In some embodiments, the pharmaceutically acceptable carrier (or vehicle) or excipient comprises one or more conventional nontoxic carrier(s) (or vehicle(s)) or excipient(s). Exemplary pharmaceutically acceptable carriers and excipients are known. Non-limiting examples include processing agents and drug delivery modifiers, such as calcium phosphate, magnesium stearate, talc, monosaccharides, disaccharides, starch, gelatin, cellulose, methyl cellulose, sodium carboxymethyl cellulose, dextrose, cyclodextrins, such as hydroxypropyl-β-cyclodextrin, polyvinylpyrrolidone, low melting waxes, ion exchange resins as well as combinations of any two or more thereof. Pharmaceutically acceptable excipients are described in “Remington's Pharmaceutical Sciences”, 18th edition, A. R. Gennaro, Ed., Mack Pub. Co. New Jersey (1991), “Pharmaceutical Formulation Development of Peptides and Proteins, S. Frokjaer and L. Hovgaard, Eds., Taylor & Francis (2000), and “Handbook of Pharmaceutical Excipients, 3rd edition, A. Kibbe, Ed., Pharmaceutical Press (2000), all of which are incorporated herein by reference.

### Methods of Treatment and Therapeutic Use

Provided herein are methods of administering and uses, such as therapeutic and prophylactic uses, of one or more of the compounds provided herein and/or compositions comprising the same as provided herein. In some embodiments, the provided therapeutic methods and uses, for example, involving administration of the molecules or compositions containing the same, to a subject having a disease, condition, or disorder associated with SURF1. In some embodiments, one or more of the compounds provided herein are administered in an effective amount to effect treatment of the disease or disorder.

Also provided herein are uses of one or more compounds as provided herein, in such methods and treatments, and in the preparation of a medicament in order to carry out such therapeutic methods. In some embodiments, the methods are carried out by administering the of one or more compounds as provided herein, to the subject having, having had, or suspected of having the disease or condition. In some embodiments, the methods thereby treat the disease or condition or disorder in the subject. Also provided herein are use of any of the compositions, such as pharmaceutical compositions provided herein, for the treatment of a disease or disorder associated with SURF1, such as for use in a treatment regimen.

In some embodiments, the one or more compounds as provided herein are used for therapeutic treatment of a subject. In some embodiments, the subject to receive therapeutic treatment displays symptoms or signs of pathology, disease, or disorder, in which treatment is administered to the subject for the purpose of diminishing or eliminating those signs or symptoms. In some embodiments, the of one or more compounds as provided herein are used for prophylactic treatment of a subject. In some embodiments, the subject to receive prophylactic treatment does not display signs or symptoms of, or displays only early signs or symptoms of, a disease, pathology, or disorder. In some embodiments, prophylactic treatment is administered for the purpose of preventing or decreasing the risk of developing the disease, pathology, or disorder. In some embodiments, the of one or more compounds as provided herein are administered in therapeutically effective amount. In some embodiments, a therapeutically effective amount is a dosage or amount of a substance sufficient to produce a desired result. In some embodiments, the desired result may comprise an objective or subjective improvement in the recipient of the dosage or amount. In some embodiments, the desired result may comprise a measurable, detectable or testable induction, promotion, enhancement, or modulation of an immune response in a subject.

In an embodiment, the disorder is Leigh Syndrome.

### Dosing

In some embodiments, the one or more compounds as provided herein and/or compositions thereof can be administered as a sole active agent. In some aspects, the specific dose level for any particular subject or patient may depend upon a variety of factors including the activity of the specific compound employed, the age, body weight, general health, route of administration, severity of the disorder, rate of excretion. The therapeutically effective amount for a given situation can be readily determined by routine experimentation and is within the skill and judgment of the ordinary clinician. The terms “therapeutically effective amount” and “therapeutically effective dose” are used interchangeably herein to refer to an amount of a compound that results in prevention or amelioration of systems in a patient or a desired biological outcome. The terms “subject” and “patient” are used interchangeably herein to refer to a mammal, such as a human, non-human primate (e.g., a baboon, an orangutan, a monkey, a gorilla), or a non-primate mammal (e.g., a mouse, a rat, a dog, a pig).

In some embodiments, a therapeutically effective dose of one or more compounds as provided herein range from at or about 0.1 μg/kg/day to at or about 20 mg/kg/day, about 10 μg/kg/day to at or about 1 mg/kg/day, or from at or about 100 μg/kg/day to at or about 1 mg/kg/day. In some embodiments, the one or more compounds as provided herein is a one or more compounds as provided herein, or a composition, such as a pharmaceutical composition, thereof. In some embodiments, any combination of the aforementioned molecules is administered at the provided doses. In some embodiments, one dose of the one or more compounds as provided herein is delivered to a subject. In some embodiments, more than one dose one or more compounds as provided herein is delivered to a subject.

In some embodiments, one or more compounds as provided herein can be administered at a recommended maximum clinical dosage and/or at lower dosages. In some embodiments, dosage levels of the any of one or more compounds as provided herein can be varied to obtain a desired therapeutic response depending, for example, on the route of administration, severity of the disease, the response of the patient. In some embodiments, the one or more compounds as provided herein is a one or more compounds as provided herein, or a composition, such as a pharmaceutical composition, thereof. In some embodiments, any combination of the aforementioned molecules is administered at the provided dosages.

In some embodiments, the one or more compounds as provided herein and/or compositions thereof can be administered in combination with one or more other agents. See, e.g., US 2008/0260737, which is incorporated herein by reference. In some embodiments, suitable agents include non-steroidal anti-inflammatory drugs (NSAIDs), glucocorticoid, prednisone, a disease-modifying anti-rheumatic drug (DMARD) (for example, hydroxychloroquine, sulfasalazine, methotrexate, leflunomide, etanercept, infliximab, rituximab, azathioprine, D-penicillamine, Gold (oral or intramuscular), minocycline, cyclosporine, retinoids, Staphylococcal protein A immunoadsorption, topical treatments (such as, for example, steroids, anthralin, calcipotriene, clobetasol, tazarotene) and the like. In some embodiments, the combination comprising a one or more compounds as provided herein can be administered as separate compositions. In some embodiments, the separate compositions may be administered at the same time or at different times. In some embodiments, the combination comprising one or more compounds as provided herein can be administered as a single dosage form containing the two or more agents.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”. The term “about” can also encompass variations, which can be up to ±5%, but can also be ±4%, 3%, 2%, 1%, etc. Whether or not modified by the term “about,” the claims include equivalents to the quantities.

The term “therapeutic treatment” refers herein to a treatment administered to a subject who displays symptoms or signs of pathology, disease, or disorder, in which treatment is administered to the subject for the purpose of diminishing or eliminating those signs or symptoms.

The term “prophylactic treatment” refers herein to a treatment administered to a subject who does not display signs or symptoms of, or displays only early signs or symptoms of, a disease, pathology, or disorder. Such treatment is administered for the purpose of preventing or decreasing the risk of developing the disease, pathology, or disorder.

The term “therapeutically effective amount” refers herein to a dosage or amount of a substance sufficient to produce a desired result. The desired result may comprise an objective or subjective improvement in the recipient of the dosage or amount. For example, the desired result may comprise a measurable, detectable or testable induction, promotion, enhancement, or modulation of an immune response in a subject.

### EXAMPLES

### Example 1

### SURF1 Screening Protocol

### Seeding Density Optimization

Shy1Δ (haploid) and Shy1Δ/Shy1Δ (diploid) along with controls (BY4741 and BY4743) were inoculated in 3 mL of YPD (yeast extract-peptone-dextrose medium) and grown overnight at 30° C. with shaking.

The overnight cultures were washed with distilled water 3 times and resuspended in water to a volume of 3 mL.

The cells were then diluted in YEP (yeast extract-peptone medium)+2% lactate to a final concentration of 0.05.

A series of 1:2 serial dilutions were performed to dilute the cultures to the following ODs: 0.05, 0.025, 0.0125, 00625 and 0.00375, respectively.

Two 384-well plates were inoculated and both plates were incubated at 30° C. One plate was read at 24 h and the other plate was read at 60 h.

At 24 h and 60 h timepoints, 25 μL of BTG (BacTiter-Glo) was added to each well and the samples were incubated for 5 min before reading the luminescence output.

In an embodiment, the data can be plotted as a scatter plot with the X-axis being the initial cell count in log scale and the Y-axis being luminescence, a sigmoidal model was used to fit the data.

### Z′ Prime Optimization

Shy1Δ (haploid) and Shy1Δ/Shy1Δ (diploid) along with controls (BY4741 and BY4743) were inoculated in 3 ml of YPD and grown overnight at 30° C. with shaking.

Overnight cultures were diluted to an OD of 0.25 and then diluted 1 to 10 to a final volume of 40 mL.

Two 384-well plates were set up with 12 columns allocated for each control and test sample (BY4741 vs. Shy1Δ); (BY4743 vs. Shy1Δ/Shy1Δ).

Plates were sealed and covered with a hydration lid filled with 6 mL of water inside a hydration chamber in the 30° C. incubator with a beaker of water for 24 h.

At the 24 h mark, 25 μL of BTG was added to each well. The samples were incubated for 10 min before reading the luminescence output.

In an embodiment, the data can be plotted as a scatter plot with the X-axis being the initial cell count in log scale and the Y-axis being luminescence, a sigmoidal model was used to fit the data.

### Pharmakon Library Screen

Shy1Δ (haploid) and Shy1Δ/Shy1Δ (diploid) along with controls (BY4741 and BY4743) were inoculated in 3 ml of YPD and grown overnight at 30° C. with shaking.

Overnight cultures were diluted to an OD of 0.25 and then diluted 1 to 10 to a final volume of 40 mL.

50 nL of 10 mM test compounds were added to columns 3-22 of a 384-well Greiner white plates using the Echo acoustic dispenser.

50 nL of DMSO was added to Columns 1-2 and 23-24 (control columns).

The density of the mutant and WT cell suspensions were adjusted to OD=0.025.

25 μL of mutant cells were added to columns 1-22, and 25 μL of WT cells were added to columns 23-24 using the EL406 bulk dispenser (or a multichannel pipette).

The plates were spun down at 250 g for 1 minute, sealed with a gas-permeable seal, and covered with water-filled microclime lid.

The plates were incubated at 30° C., and incubated for 24 h.

After 24 h, the seals were removed, and the plates were allowed to incubate at room temp for 15 minutes.

25 μL/well of room temp equilibrated BacTiter-Glo solution was added using the EL406 bulk dispenser.

The plates were briefly shaken for 2 min and incubated for 10 min.

The luminescence output of the plate was read using the Envision plate reader.

A list of hits was generated. The top 2% of the hits were chosen for further analysis.

### Example 2

The most common recurring SURF1 mutation is c.312_320del10insAT. It mirrors the effect of SURF1 whole-gene deletion and results in Leu105X.

Leu105X SURF1 yeast avatars were generated based on the protocols developed in Lao et al., 2019. Rescue plasmids encoding yeast SURF1 with and without the two deletion mutations were ordered from a gene synthesis vendor The growth rate under respiratory conditions of these SHY1 sensitized mutant yeast cells was assessed relative to a positive control wildtype strain and negative control SURF1 whole-gene deletion strain. A high-throughput growth-based assay was optimized, and then multiple yeast strains were deployed in a drug repurposing screening campaign.

The following haploid strains were generated and tested using promoters of various strengths:
1) SHY (positive control)
2) shy1Δ (whole-gene deletion negative control)
3) shy1ΔL105X (0.2× promoter)
4) shy1ΔL105X (1× promoter)
5) shy1ΔL105X (2× promoter)
6) shy1ΔG137E (missense negative control)

By tuning the levels of SHY1 variant expression, a haploid strain with an intermediate, rescuable growth defect was used in the drug repurposing screen.

### Example 3

### Effect of Sertaconazole in Human Neurons Carrying Mutations in the Gene SURF1

Hit compounds were filtered and selected to test their effect on neuronal morphogenesis CRISPR/Cas9 engineered isogenic SURF1 mutant iPSCs on which neural commitment via overexpression of NGN2 was forced were used. The impact of the selected hit compounds on neurite outgrowth capacity on these early induced neurons was measured. Sertaconazole was identified as being able to carry out a dose-dependent rescue of neurite outgrowth capacity (FIG. 28A, FIG. 28B, and FIG. 28C).

Sertaconazole ameliorates neuromorphogenesis defects in SURF1-mutant neurons. FIG. 28A depicts quantification of total neurite area in SURF1-mutant neurons. Each value represents the total neurite area in each well of a 96-well plate. Significant values were calculated using the non-parametric Mann-Whitney test. ****p<0.0001 showed defective neurite development in SURF1-mutant cells. FIG. 28B depicts quantification of total neurite area in SURF1-mutant neurons treated with either the DMSO vehicle or Sertaconazole at different concentrations. Neurite area was measured 5 days after induction of NGN2 overexpression, using high-content image analysis of MAP2-positive stained neuronal cells. Each value represents the total neurite area in each well of a 96-well plate. Significant values were calculated using the non-parametric Mann-Whitney test **p<0.01 showed correction of defective neurite development in SURF1-mutant cells. FIG. 28C depicts representative images of SURF1 mutant neurons treated with either the DMSO vehicle or Sertaconazole.

### Example 4

### Effect of Sertaconazole in Brain Organoids Carrying Mutations in the Gene SURF1

SURF1-mutant midbrain organoids were employed, which showed key disease-specific features, such as increased lactate released in the media, decreased number of tyrosine hydroxylase-positive dopaminergic neurons and aberrant neuronal branching, as seen by altered dendrites (positive for MAP2) and altered axons (positive for SMI312). It was determined that Sertaconazole reduced the aberrant release of lactate in the media, increased the amount of TH-positive dopaminergic neurons, and rescued the overall neuronal branching organization (FIG. 29A, FIG. 29B, FIG. 29C, and FIG. 29D).

Sertaconazole ameliorates neuromorphogenesis and lactate defects in SURF1-mutant midbrain organoids. FIG. 29A depicts lactate released in the media by SURF1-mutant midbrain organoids compared to isogenic control organoids. The amount of lactate was quantified using a commercial kit. Significant values were calculated using the non-parametric Mann-Whitney test. ***p<0.001 showed aberrant increase of lactate release by SURF1-mutant organoids. FIG. 29B depicts quantification of lactate in the media of SURF1-mutant midbrain organoids that were treated with either DMSO vehicle or 0.1 μM Sertaconazole for 1 week Media was collected and analyzed for lactate content using a commercial kit. Significant values were calculated using the non-parametric Mann-Whitney test. ****p<0.0001 showed that Sertaconazole treatment decreases the aberrant amount of lactate released to the media. FIG. 29C depicts that Sertaconazole treatment corrected the increased the amount of neuronal processes in human midbrain organoids carrying a mutation in the gene SURF1 causative of Leigh syndrome. SURF1-mutant midbrain organoids were treated with either the DMSO vehicle or 0.1 μM Sertaconazole for 1 week. The organoids were then fixed and stained with antibodies against Tyrosine Hydroxylase (TH) (a marker of dopaminergic neurons), MAP2 (a marker for neuronal dendrites), and SMI312 (a marker for neuronal axons). Significant values were calculated using the non-parametric Mann-Whitney test. The increase in TH neurons was evident but did not reach statistical significance. *p<0.05 showed that Sertaconazole treatment corrected the increased the amount of neuronal processes in human midbrain organoids carrying a mutation in the gene SURF1 FIG. 29D depicts representative images of SURF1-mutant midbrain organoids treated with either the DMSO vehicle or Sertaconazole. Sertaconazole can represent an effective repositionable drug for children affected by Leigh syndrome.

### Example 5

### Experiment 1: Fibroblast Cell Line GM28010 (Single-Compound Treatments)

Fibroblast cell line GM28010 is a primary human fibroblast cell line obtained from patients with SURF1 Leigh syndrome. It was purchased from the Coriell Cell Repositories (Camden, NJ, repository number GM28010). Cell line GM00038 was used as a healthy control for GM28010 and is a primary human fibroblast purchased from the Coriell Cell Repositories (Camden, NJ, repository number GM00038).

Compounds tested are shown in Table 1.

TABLE 1

| | Compound | Screen identified |
|---|---|---|
| 1 | Glabridin | TargetMol |
| 2 | Incyclinide | Pharmakon |
| 3 | Losmapimod | Personal communication |
| 4 | Miltefosine | TargetMol, Pharmakon, ReFrame |
| 5 | PF-06447475 | TargetMol |
| 6 | Posaconazole | TargetMol, Pharmakon, ReFrame |
| 7 | Sertaconazole | TargetMol, Pharmakon |
| 9 | Telaprevir | TargetMol |
| 10 | Poziotinib | TargetMol |
| 11 | Ginsenoside Z R1 | TargetMol |
| | (Zinglibroside) | |
| 12 | AM281 | TargetMol |

### Study Design—48 Hour Compound Treatment

### Cell Culture and Treatment

Fibroblasts were grown in glucose according to recommendation.

Cells were plated in XF96 plates in culture medium and were plated at 10,000 cells/well.

The day after plating, cells were treated with the compounds in Table 1. The treatments were completed in glucose medium and galactose medium. The compounds were tested at for 48 hours. The compounds were incubated at (37 C, 5% CO2) until the day of the experiment.

*Fibroblasts were run at passage numbers: GM28010 SURF1 Fibroblasts P5; GM00038F controls P9.

### Seahorse Conditions

Assay Medium: 10 mM glucose or galactose DMEM supplemented with 2 mM glutamine and 1 mM pyruvate.
Injection Ports for basal glucose: Port A: 2 μM oligomycin; Port B: 2.5 μM FCCP; Port C: 4 μM FCCP; and Port D: 2 μM rotenone and antimycin A.

Data were normalized to cell number per well after the run. The Hoechst-stained nuclei were counted with the Operetta High-Content Imager.

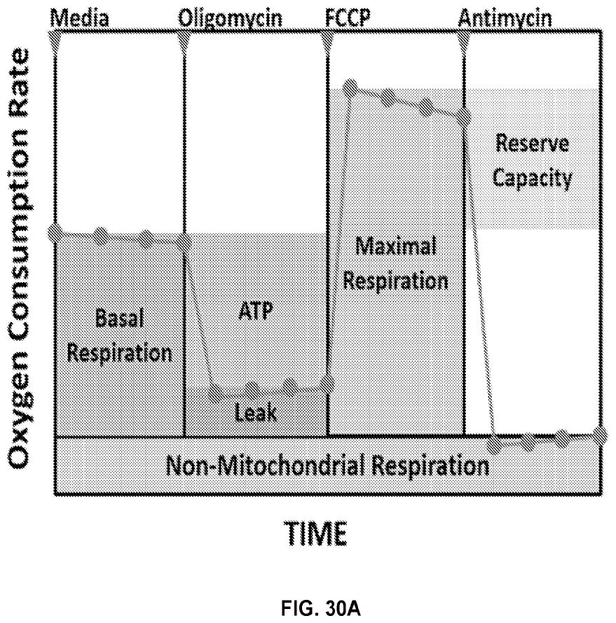

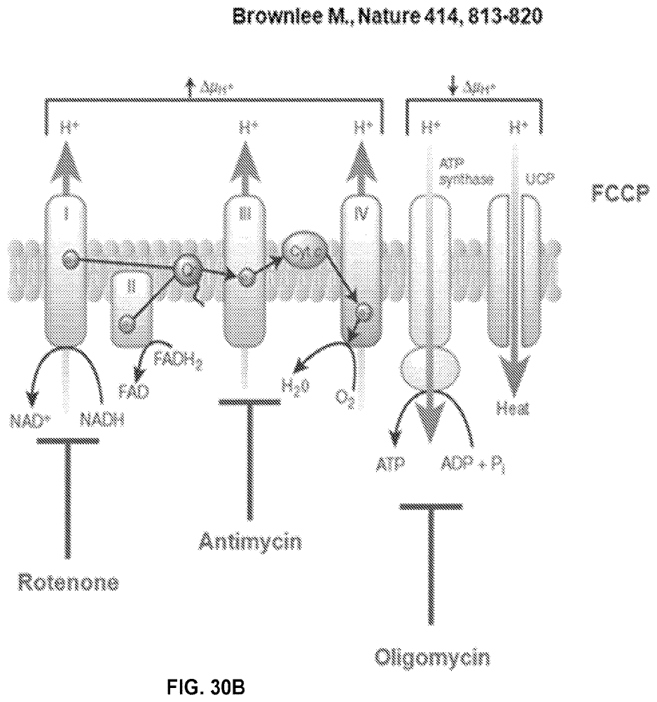

### Mitochondrial “Stress Test” Overview (FIG. 30A and FIG. 30B)

Basal Oxygen Consumption (OCR): This reflects the cellular oxygen usage under basal conditions, encompassing both energy production (ATP turnover) and background oxygen leak from the mitochondria.
Oligomycin Injection: Oligomycin is a compound that inhibits ATP synthase, an enzyme crucial for ATP production within mitochondria.
Oligomycin-resistant Respiration: After oligomycin injection, minimal oxygen consumption persists, representing non-ATP linked oxygen leak from the mitochondria, often termed proton leak.
ATP-linked Respiration: This value is derived by subtracting the oligomycin-resistant respiration from the basal OCR. It signifies the oxygen consumption specifically linked to ATP production through mitochondrial oxidative phosphorylation.
Mitochondrial Bioenergetic Efficiency: This parameter reflects the percentage decrease in oxygen consumption observed after oligomycin injection, indicating the mitochondria's efficiency in utilizing oxygen for ATP generation.
FCCP Injection: FCCP is a chemical that disrupts the link between mitochondrial electron transport and ATP production.
Maximal Respiration: Following FCCP injection, the highest oxygen consumption rate is measured, representing the maximum capacity of the mitochondria to utilize oxygen for respiration.
Spare Respiratory Capacity: This parameter is calculated by subtracting basal respiration from maximal respiration. It reflects the reserve respiratory capacity available for the cells to respond to increased energy demands.
Antimycin A and Rotenone Injection: These compounds inhibit different complexes within the electron transport chain, completely blocking mitochondrial respiration.
Non-mitochondrial Respiration: The low level of oxygen consumption measured after inhibiting the electron transport chain represents cellular respiration independent of mitochondria.
ATP Production Rate: This value is indirectly calculated using known metabolic relationships between oxygen consumption, proton leak, and ATP turnover, based on the measured OCR and ECAR values obtained during the assay.

### EXPERIMENTS

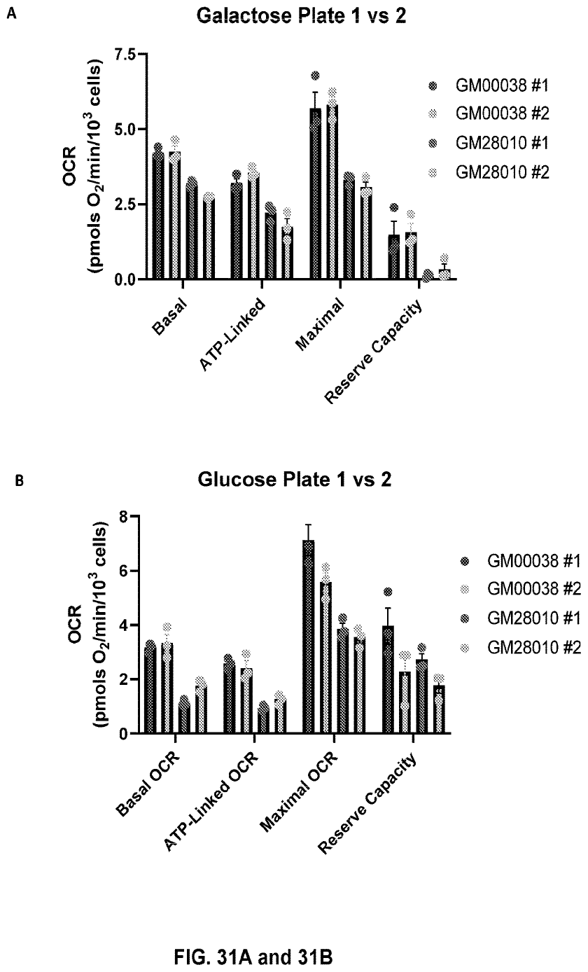

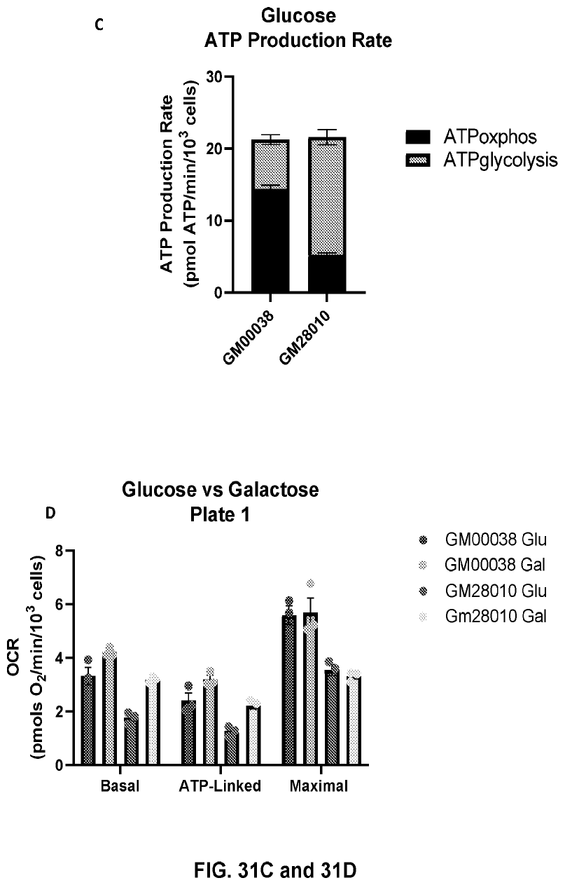

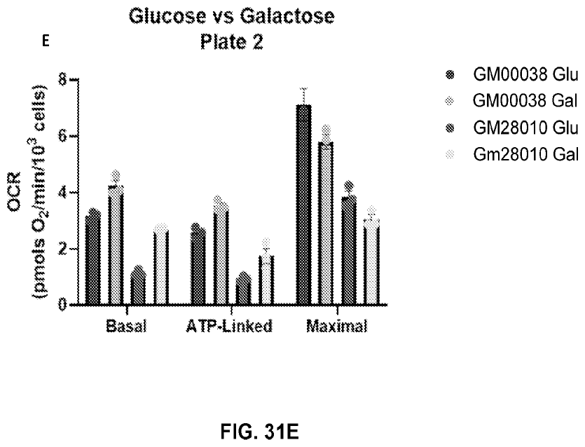

FIGS. 31A, 31B, 31C, 31D, and 31E show the compound treatment plate controls. The SURF1 mutant cells display a severe respiratory defect compared to a control cell line (GM00038F). There is a decrease in all respiratory parameters and an increase in ATP production through glycolysis to compensate for the respiratory defect. The cells responded similarly on the four different assay plates (2 glucose plates and 2 galactose plates). However, the control line had a slightly lower response to FCCP and, therefore, maximal respiration on plate #2 in the glucose-culture cells. In these cells, galactose culturing increased basal respiration, but an increase in maximal respiration in these experiments was not detected.

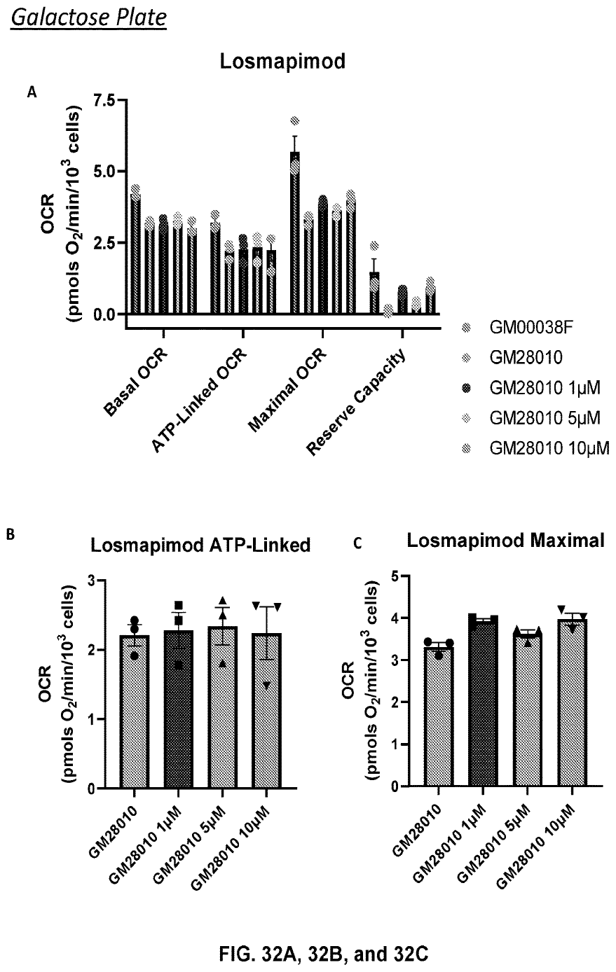

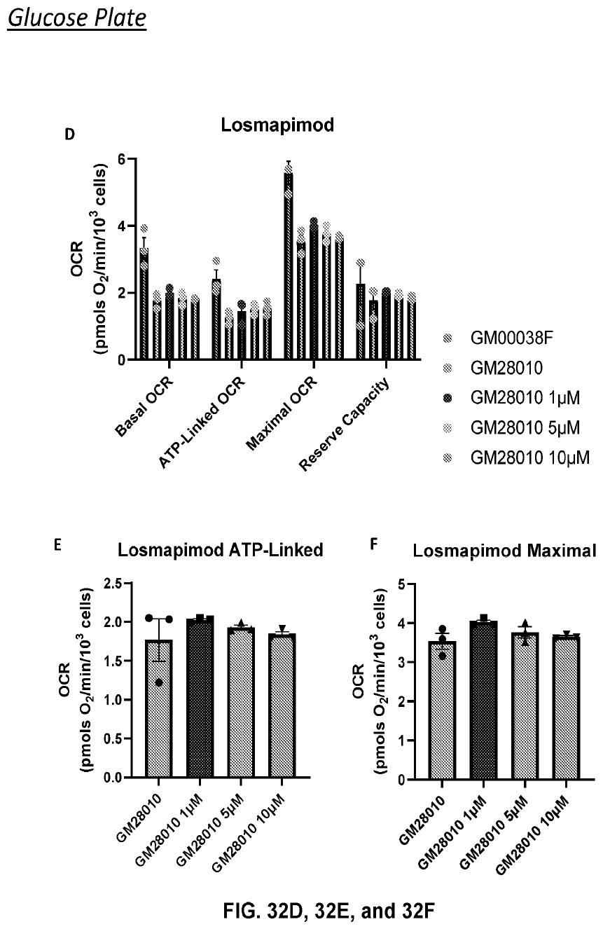

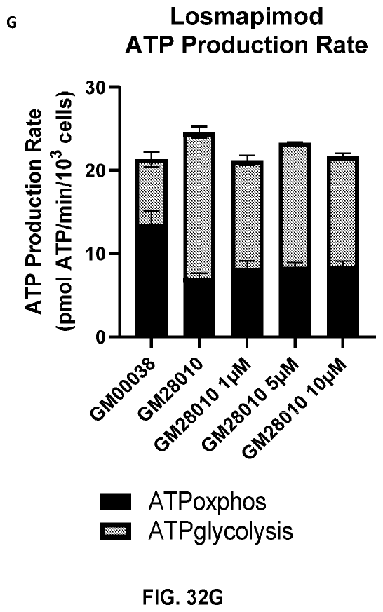

FIGS. 32A, 32B, 32C, 32D, 32E, 32F, and 32G show that treatment with losmapimod for 48 hours displayed 12-15% increases in respiration in ATP-linked and maximal respiration in both glucose and galactose-cultured cells. There was also a decrease in ATP production through glycolysis. Assays with longer compound treatments or an expanded dose-response could be performed.

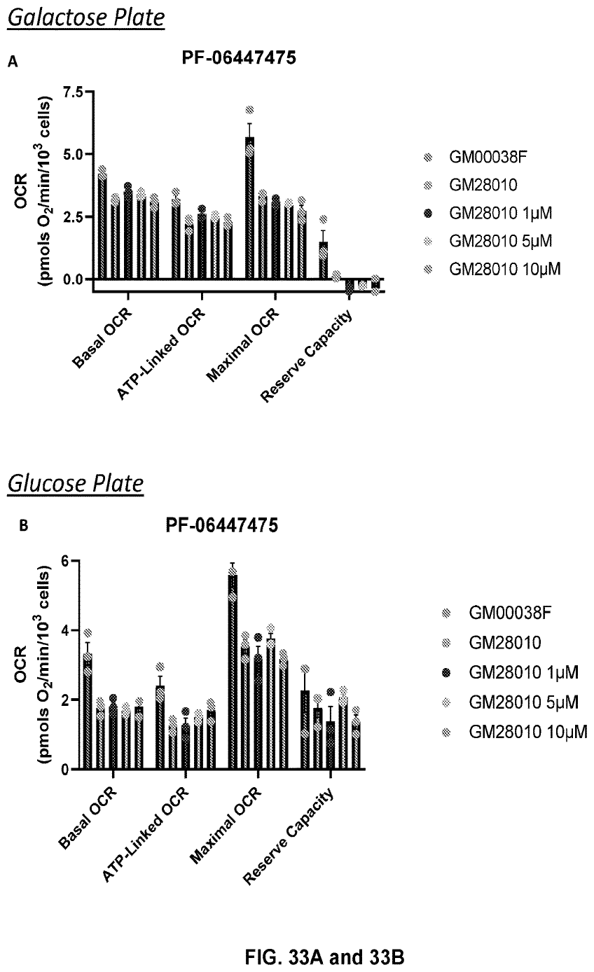

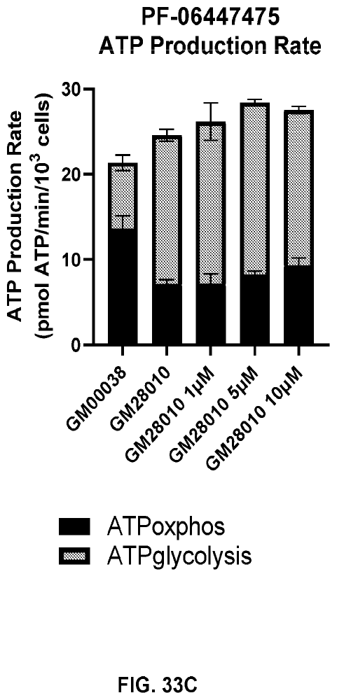

FIGS. 33A, 33B, and 33C show that treatment with PF-06447475 for 48 hours did not result in an increase in maximal respiration in either glucose or galactose conditions. There was an increase in ATP production through both OXPHOS and glycolysis with this compound.

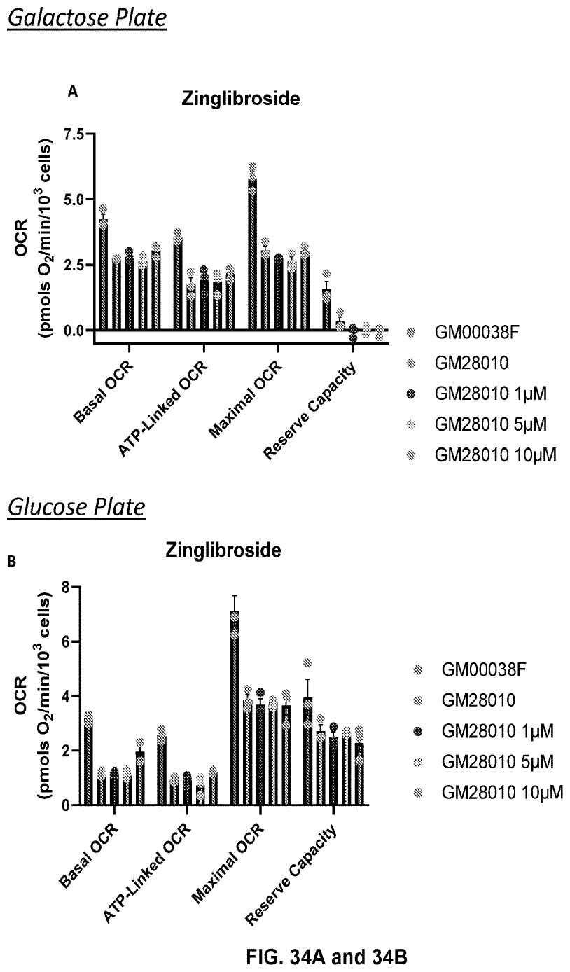

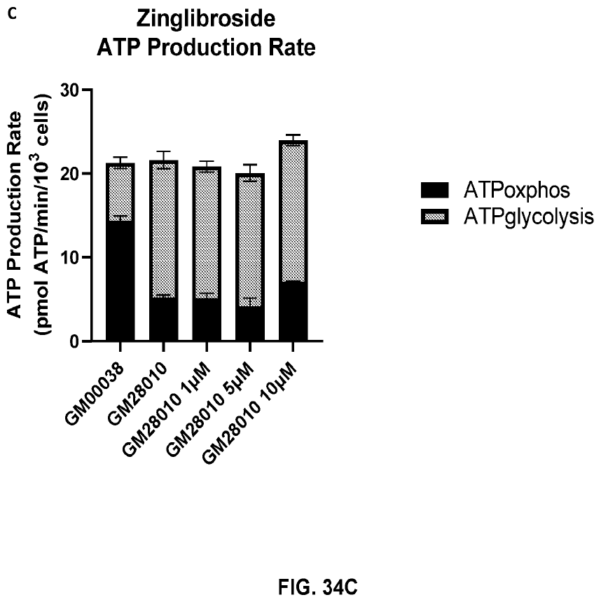

FIGS. 34A, 34B, and 34C show that treatment with Ginsenoside Z R1 (Zinglibroside) for 48 hours did not result in changes to maximal respiration in either glucose or galactose conditions. There was an increase in ATP production through OXPHOS and glycolysis with the 10 μM concentration.

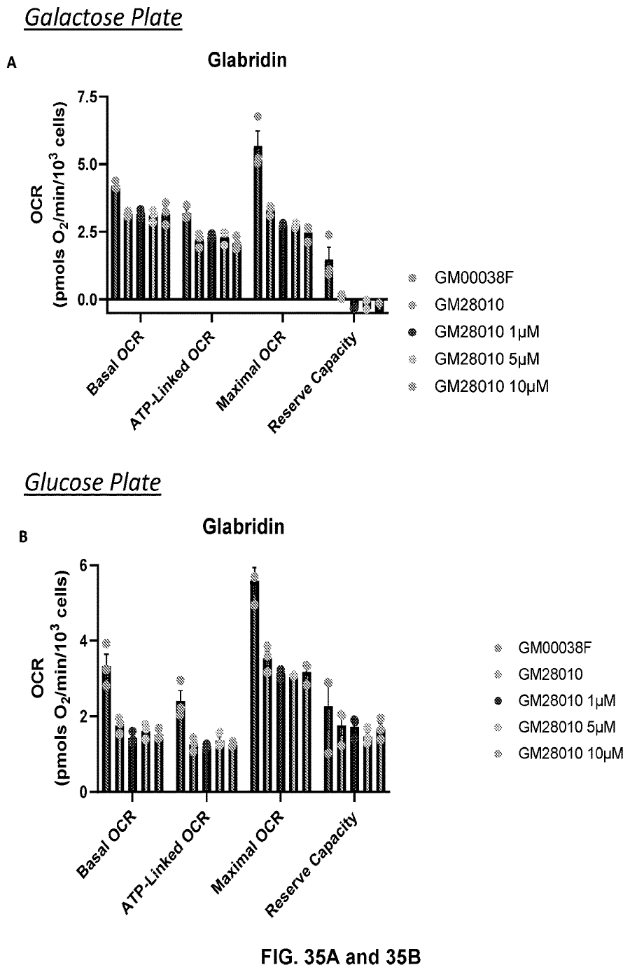

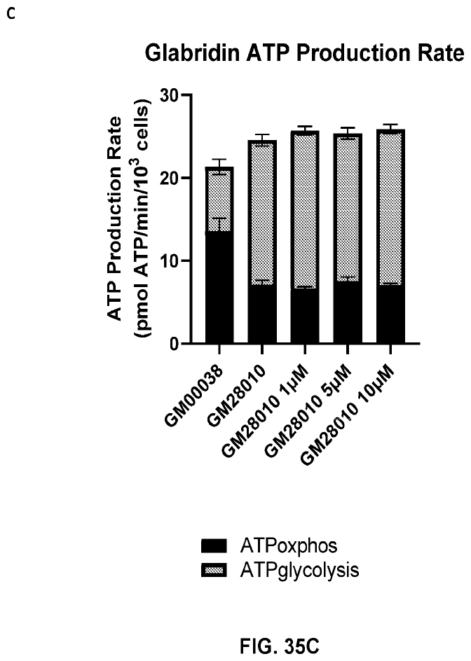

FIGS. 35A, 35B, and 35C show that treatment with Glabridin for 48 hours did not result in major changes to respiration in either glucose or galactose conditions.

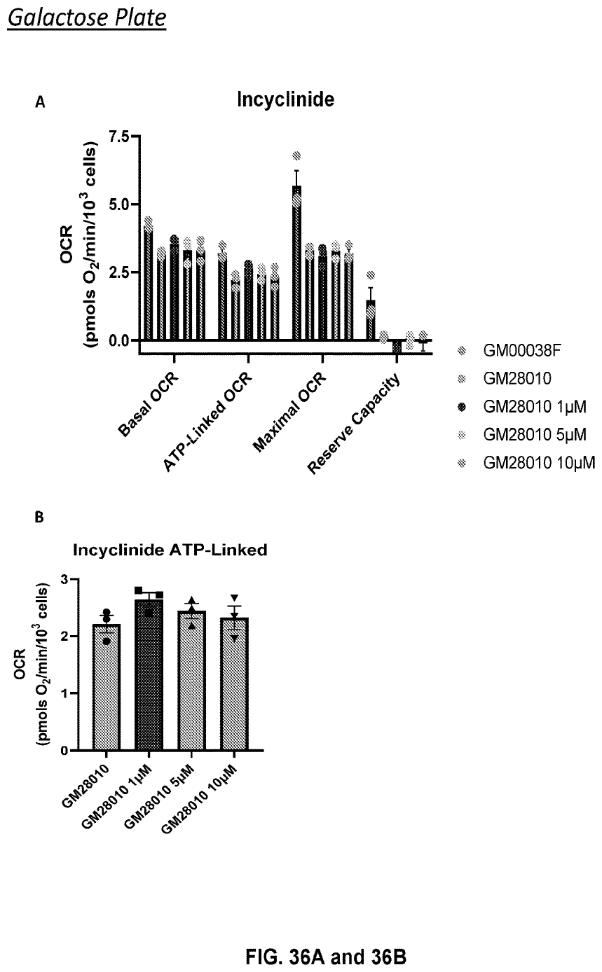

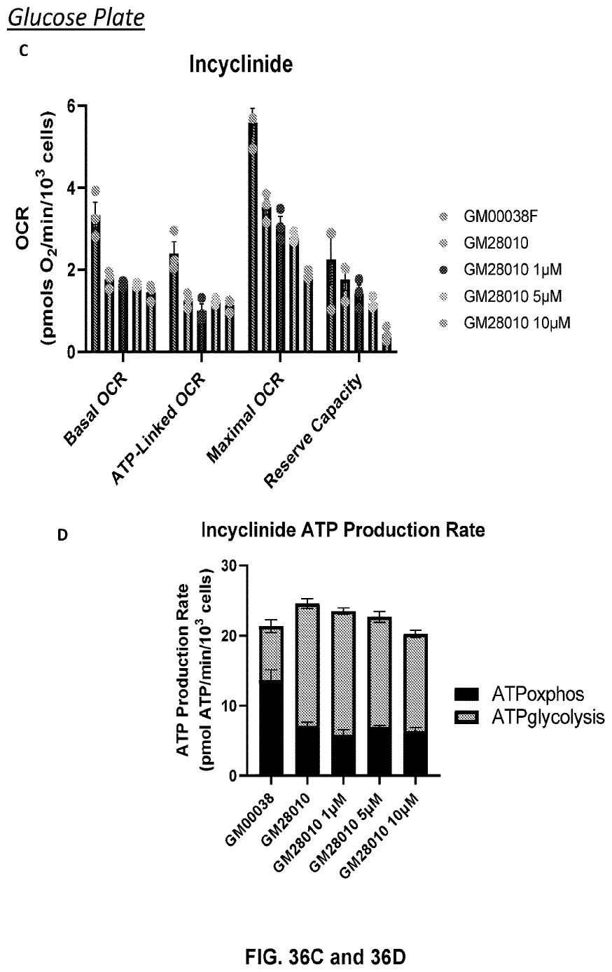

FIGS. 36A, 36B, 36C, and 36D show that treatment with incyclinide for 48 hours showed some increase to basal respiration and ATP-linked respiration in galactose-cultured cells. This compound showed some toxicity in glucose-cultured cells with a dose-dependent decrease in all respiratory parameters measured.

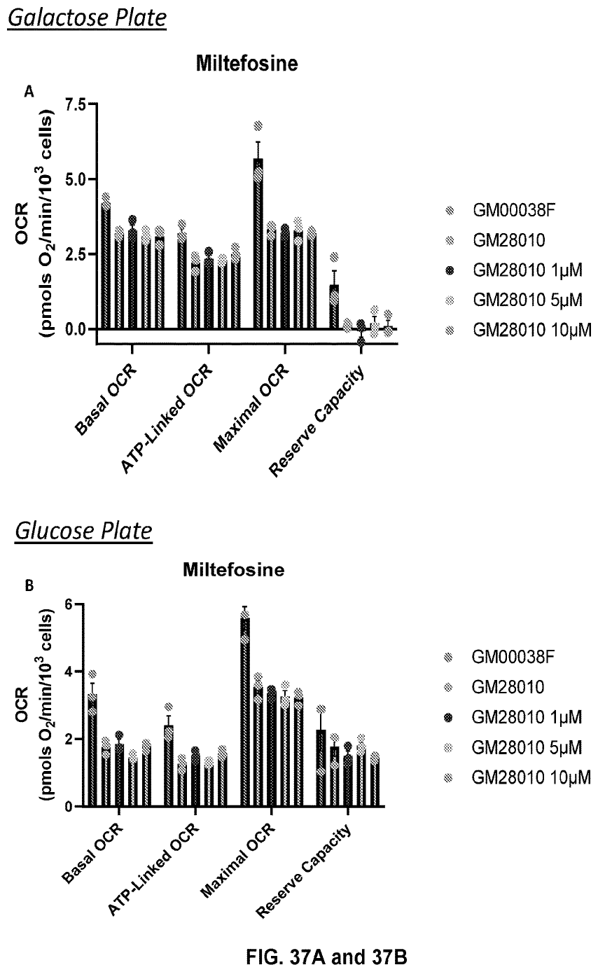

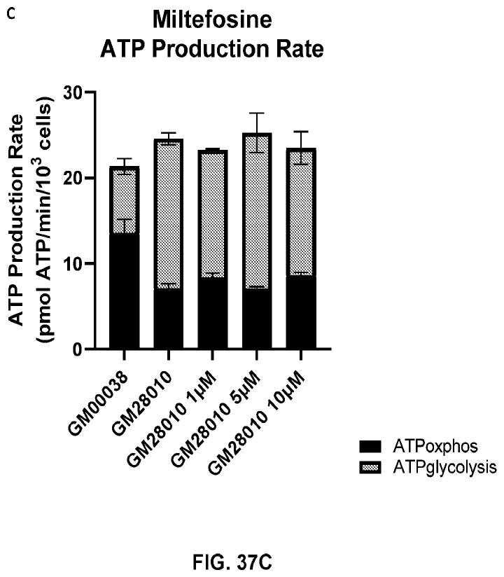

FIGS. 37A, 37B, and 37C show that treatment with miltefosine for 48 hours did not result in major changes to respiration in either glucose or galactose conditions.

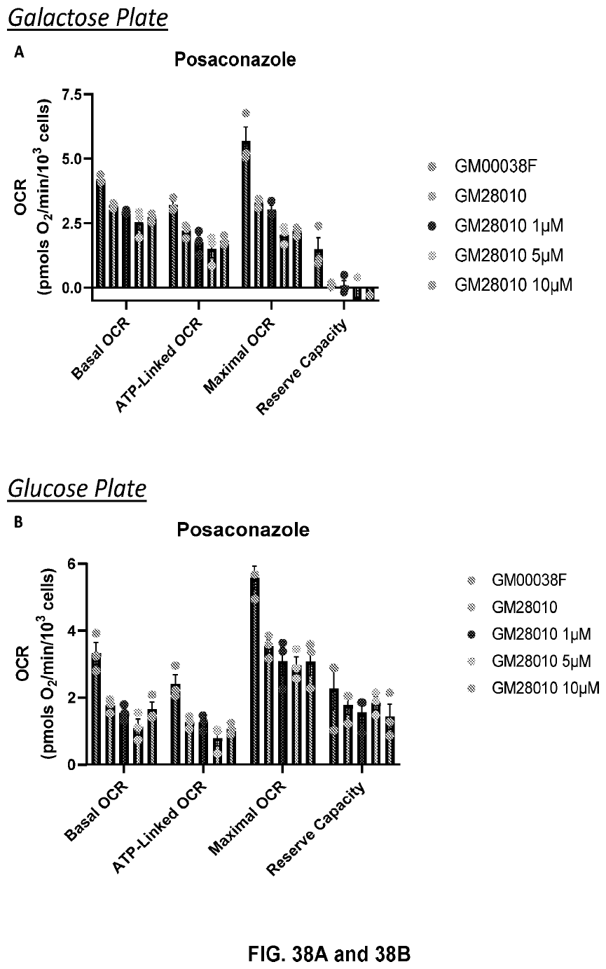

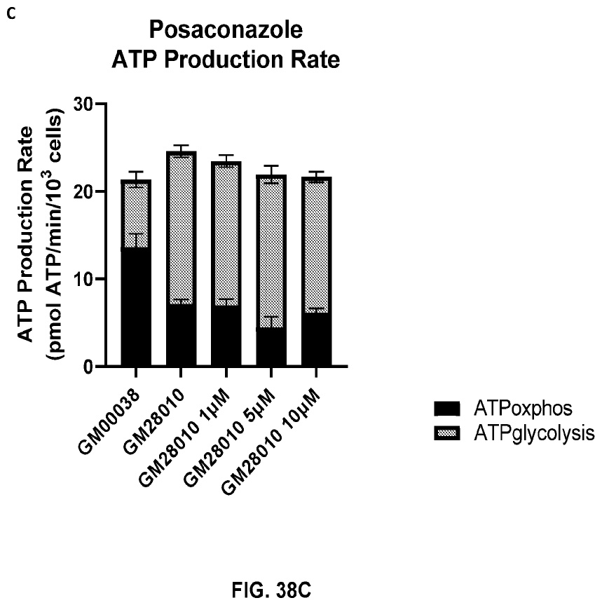

FIGS. 38A, 38B, and 38C show that treatment with posaconazole for 48 hours did not result in major changes to respiration in either glucose or galactose conditions. At higher concentrations, it decreased respiration.

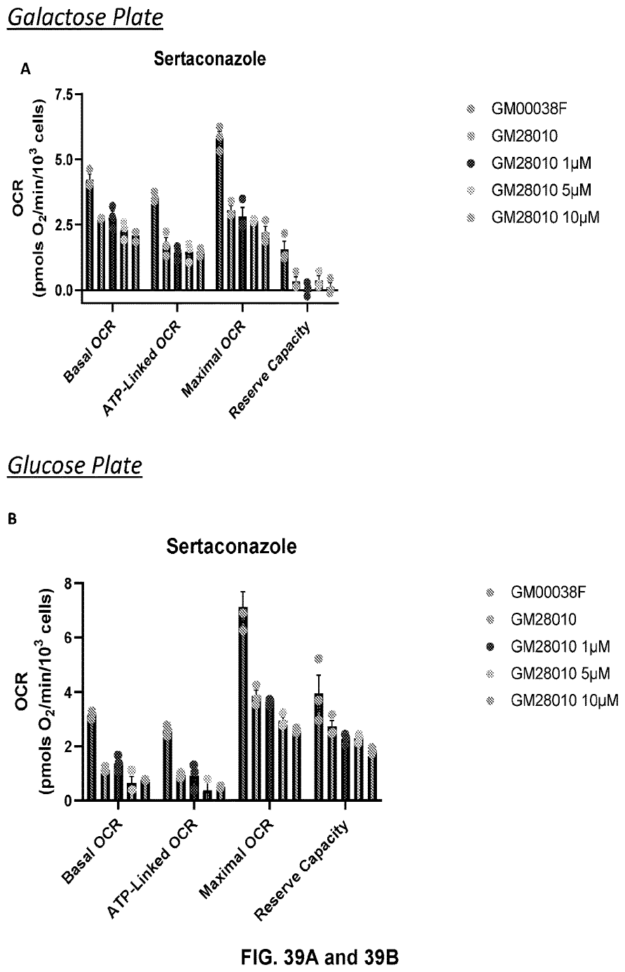

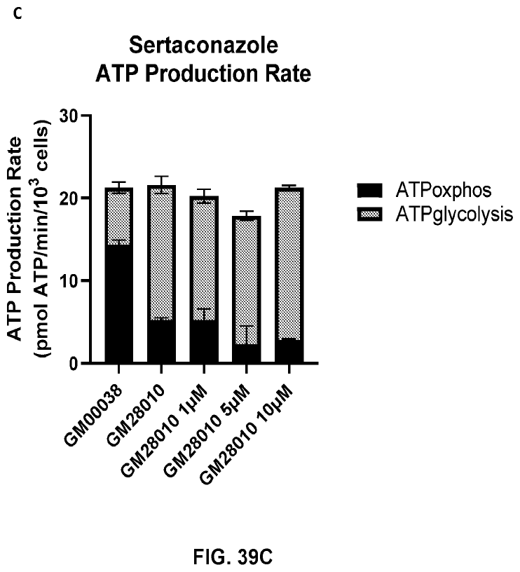

FIGS. 39A, 39B, and 39C show that treatment with sertaconazole for 48 hours did not result in major changes to respiration in either glucose or galactose conditions.

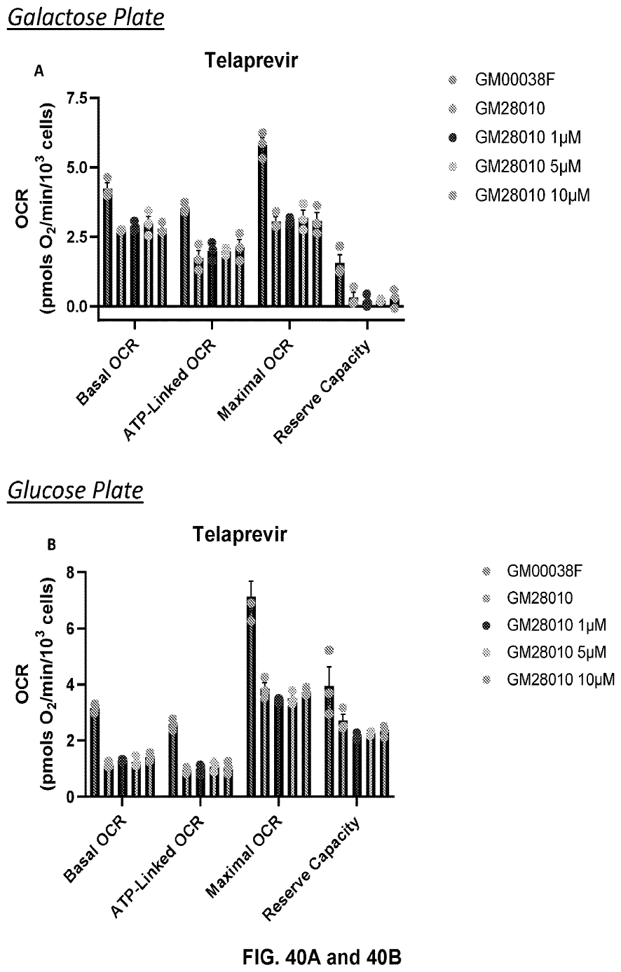

FIGS. 40A, 40B, and 40C shows that treatment with telaprevir for 48 hours did not result in major changes to respiration in either glucose or galactose conditions.

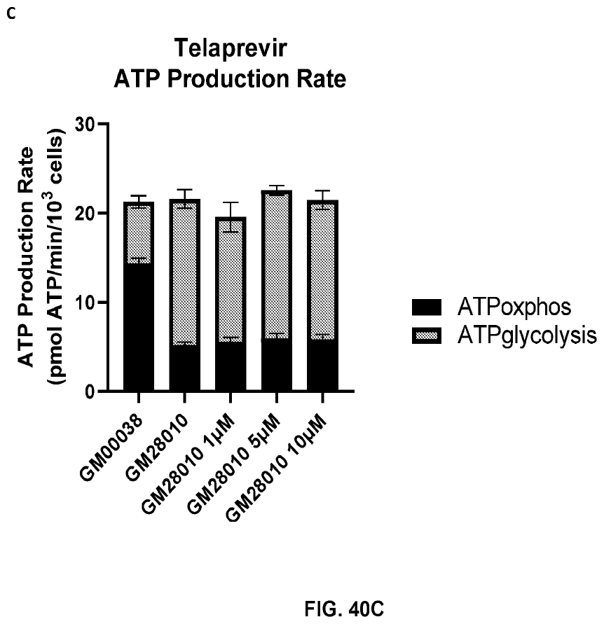

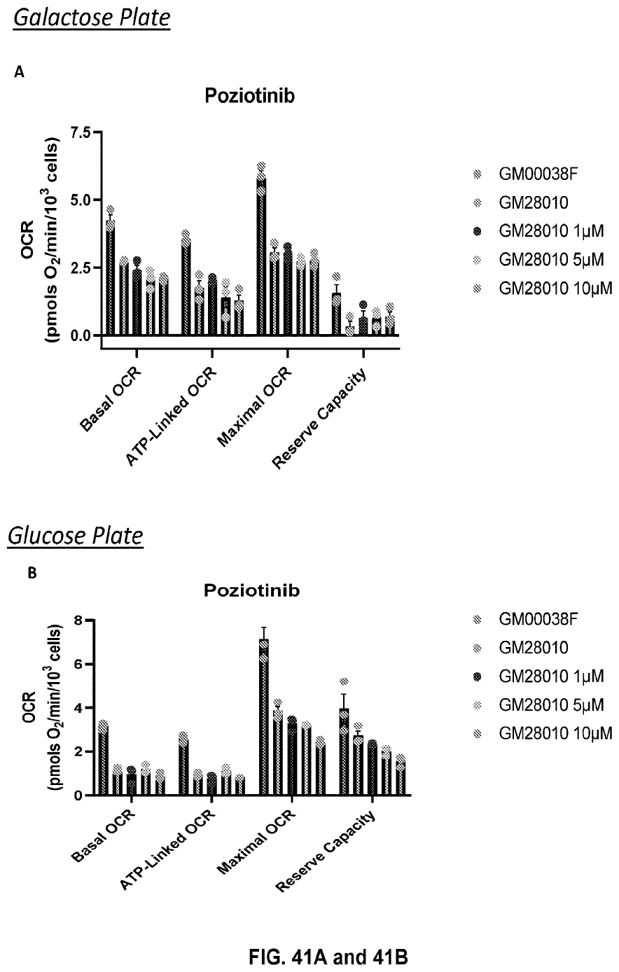

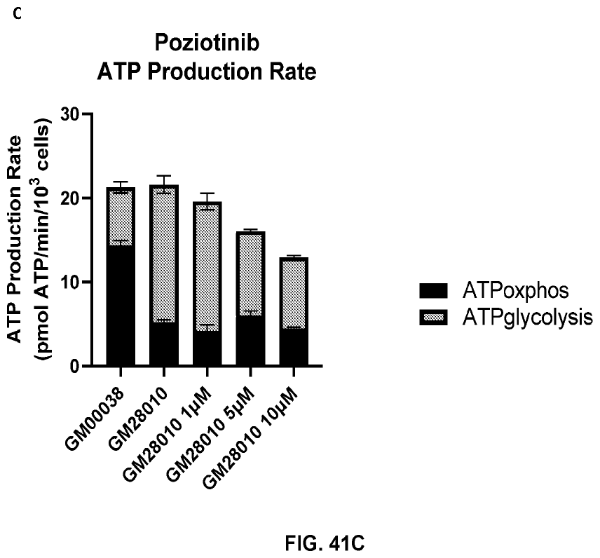

FIGS. 41A, 41B, and 41C show that treatment with poziotinib for 48 hours did not result in major changes to respiration in either glucose or galactose conditions. It did decrease ATP production through glycolysis. This compound showed some toxicity in glucose-cultured cells with a dose-dependent decrease in maximal respiration and glycolysis.

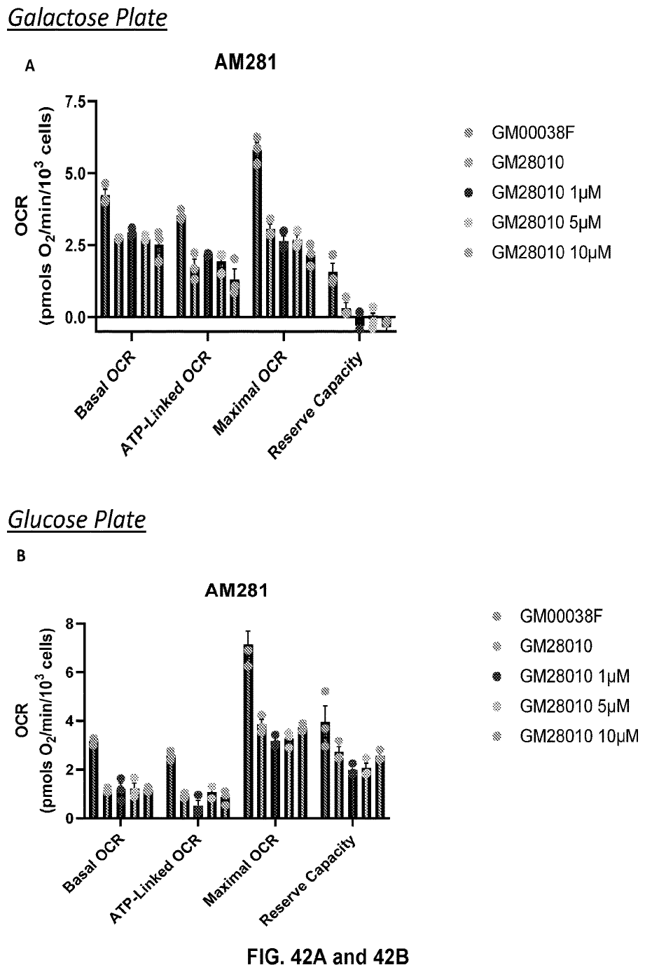

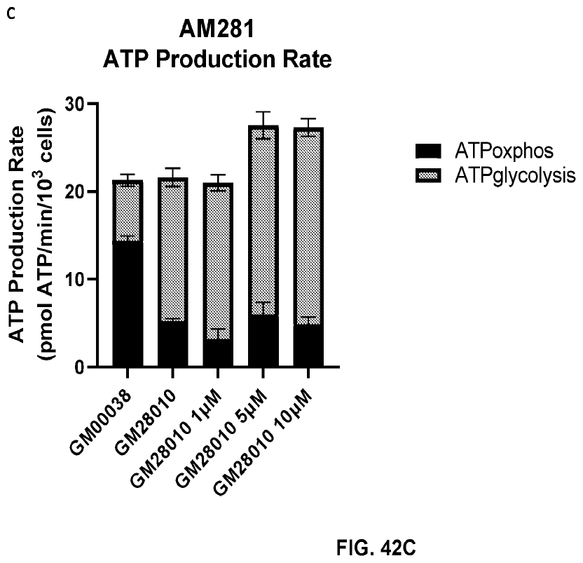

FIGS. 42A, 42B, and 42C show that treatment with AM281 for 48 hours did not result in major changes to respiration in either glucose or galactose conditions. There was an increase in ATP production primarily through glycolysis at the 5 and 10 μM concentrations.

### Results

Fibroblasts provided the characteristic response to injected compounds during the Seahorse assay.

Fibroblasts responded to galactose culturing for 48 hours by increasing basal respiration rates but not maximal respiration.

GM28010 SURF1 mutant fibroblasts had lower respiration rates compared with GM0038F control fibroblasts in both glucose and galactose culturing conditions. Respiration was decreased by approximately 50% or more in galactose and glucose-cultured cells. SURF1 mutant cells compensated with increased glycolysis.

Experimental compounds did not always have the same respiratory effects in glucose and galactose-cultured cells.

The compound that showed the most interesting results on respiration was losmapimod. Increased respiration in both glucose and galactose culture conditions. Respiration was increased up to 15%. Experiments with increasing the treatment time and increasing the dose-range could be performed.

### Example 6

### Experiment 2: Fibroblast Cell Line GM28010 (Combination Treatments and Single-Compound Treatments)

GM28010 is a primary human fibroblast cell line that was obtained from patients with SURF1 Leigh syndrome. It was purchased from the Coriell Cell Repositories (Camden, N.J., repository number GM28010).

GM00038 is a primary human fibroblast cell line purchased from the Coriell Cell Repositories (Camden, N.J.; repository number GM00038). GM00038 was used as a healthy control.

The compounds were used in this experiment are shown in Table 2.

TABLE 2

| Compound | Screen identified |
|---|---|
| Losmapimod | Personal communication |
| PF-06447475 | TargetMol |
| Ginsenoside Z R1 (Zinglibroside) | TargetMol |
| Methylene Blue | Personal communication |
| cysteamine bitartrate | Personal communication |

The experimental design is shown in Table 3.

TABLE 3

| | | Ginsenoside | PF- | | |
|---|---|---|---|---|---|
| | Losmapimod | Z R1 | 06447475 | cysteamine | Methylene |
| | (1 μM) | (10 μM) | (10 μM) | bitartrate | blue |
| 1 | + | + | + | | |
| 2 | + | + | | | |
| 3 | | + | + | | |
| 4 | + | | + | | |
| 5 | | | | + (1 μM) | |
| 6 | | | | + (5 μM) | |
| 7 | | | | + (10 μM) | |
| 8 | | | | | + (1 μM) |
| 9 | | | | | + (5 μM) |
| 10 | | | | | + (10 μM) |

### Study Design—48 Hour Compound Treatment

### Cell Culture and Treatment

Fibroblasts were grown in glucose according to recommendation. Cells were plated in XF96 plates in culture medium and plated at 10,000 cells/well. The day after plating, cells were treated with compounds. Treatments completed in glucose medium Compounds were tested at for 48 hours. The compounds were incubated at (37 C, 5% CO₂) until the day of the experiment.

The fibroblasts were run at passage numbers: GM28010 SURF1 Fibroblasts P5.

### Seahorse Conditions

The Assay Medium was 10 mM glucose or galactose DMEM supplemented with 2 mM glutamine and 1 mM pyruvate. Injection Ports for basal glucose: Port A: 2 μM oligomycin; Port B: 2.5 μM FCCP; Port C: 4 μM FCCP; and Port D: 2 μM rotenone and antimycin A. The Data were normalized to cell number per well after the run. The Hoechst-stained nuclei were counted with a Operetta High-Content Imager.

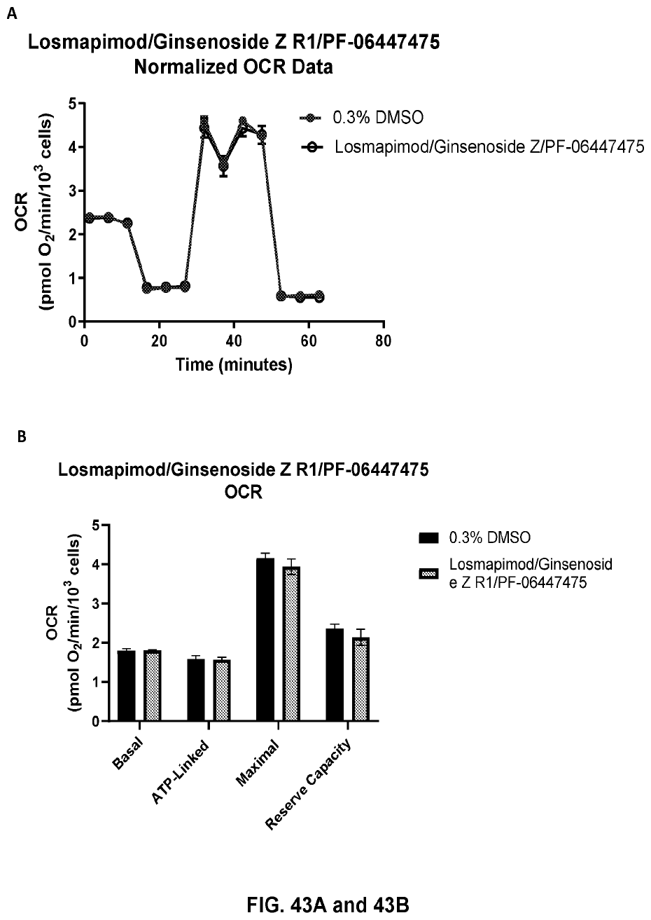

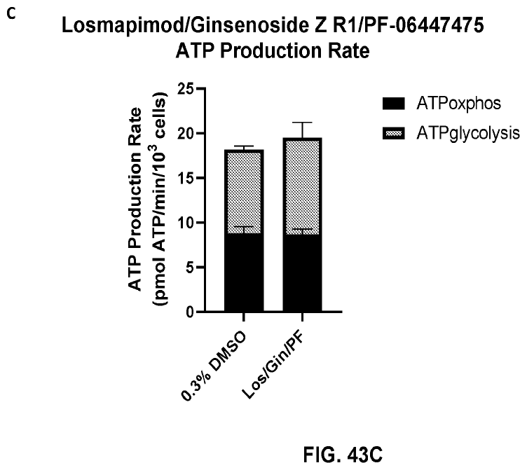

FIGS. 43A, 43B, and 43C show that treatment with the triple combination of compounds did not alter oxygen consumption in GM28010 cells. 1 μM Losmapimod/10 μM Ginsenoside Z R1/10 μMPF-06447475 were used.

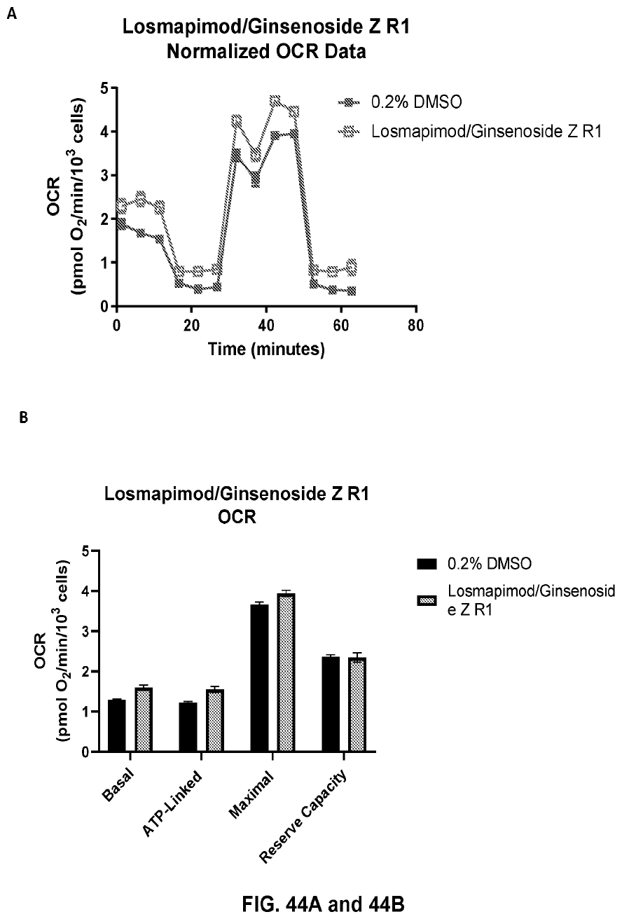

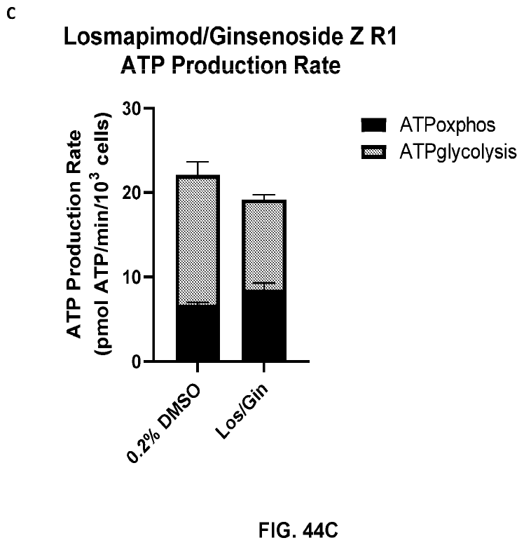

FIGS. 44A, 44B, and 44C show that treatment with the double combination of losmapimod and ginsenoside slightly increased respiration and ATP production rate through OXPHOS while decreasing ATP production through glycolysis. 1 μM Losmapimod/10 μM Ginsenoside Z R1 were used.

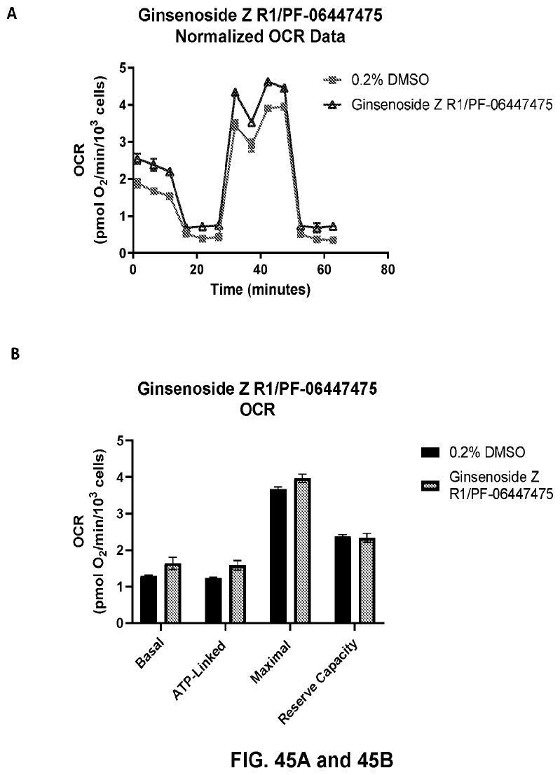

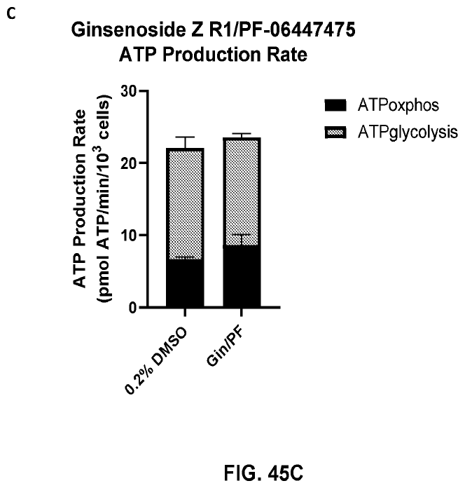

FIGS. 45A, 45B, and 45C show that treatment with the double combination of ginsenoside and PF-06447475 slightly increased respiration and ATP production rate through OXPHOS while maintaining total ATP production rates. 10 μM Ginsenoside Z R1/10 μMPF-06447475 were used.

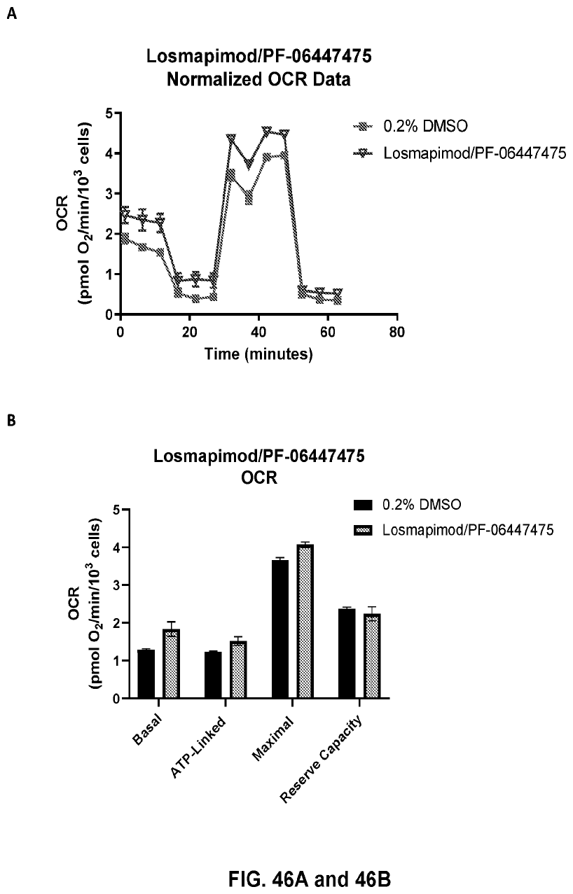

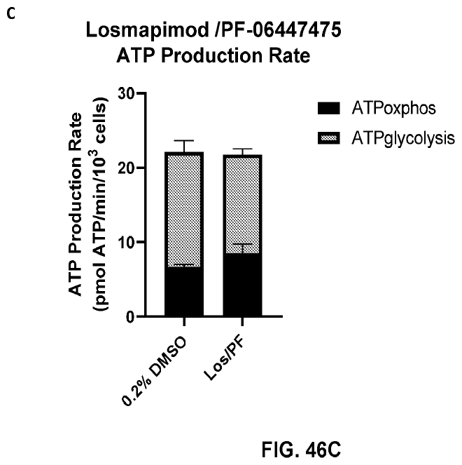

FIGS. 46A, 46B, and 46C show that treatment with the double combination of losmapimod and PF-06447475 slightly increased respiration and ATP production rate through OXPHOS while maintaining total ATP production rates. 1 μM Losmapimod/10 μMPF-06447475 were used.

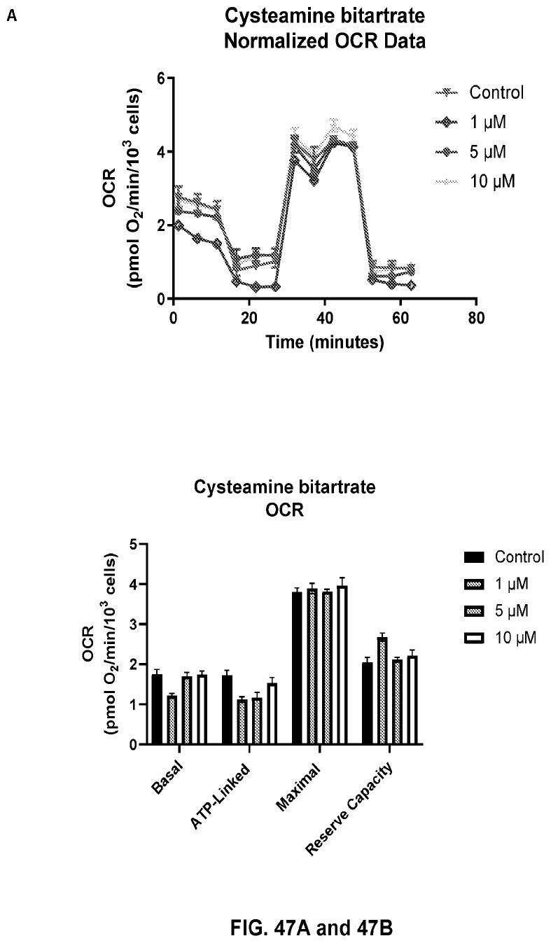

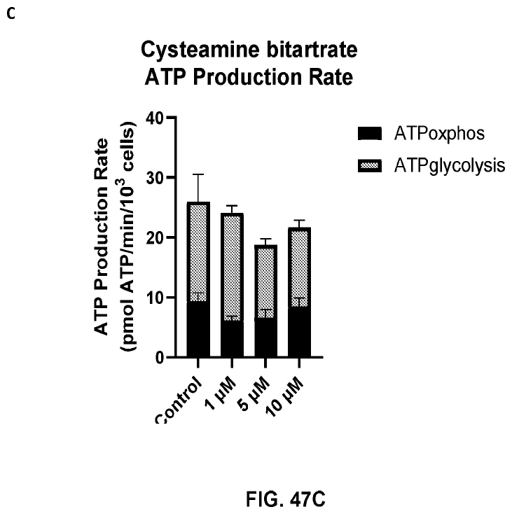

FIGS. 47A, 47B, and 47C show that treatment with cysteamine bitartrate did not alter oxygen consumption rates in GM28010 cells. ATP production rate through glycolysis was decreased with the higher concentrations of cysteamine bitartrate. Cysteamine Bitartrate was used.

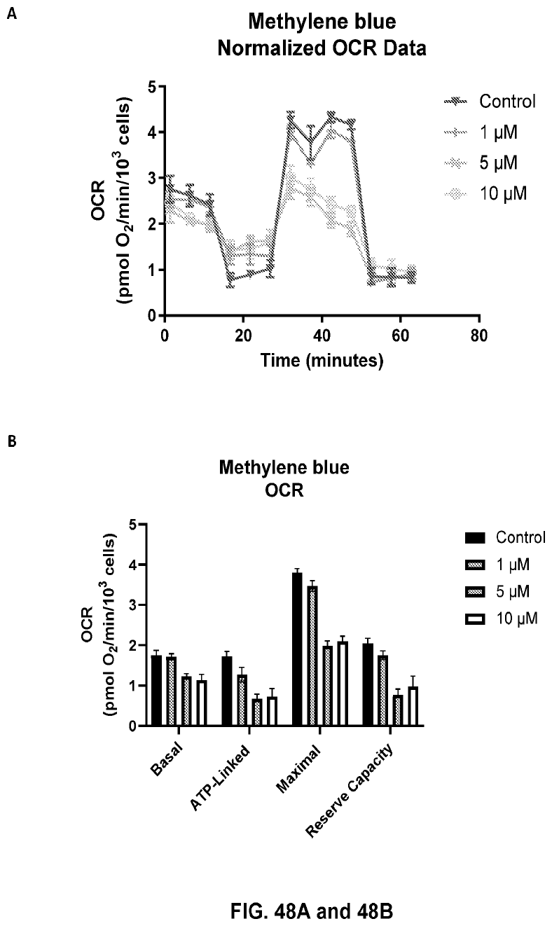

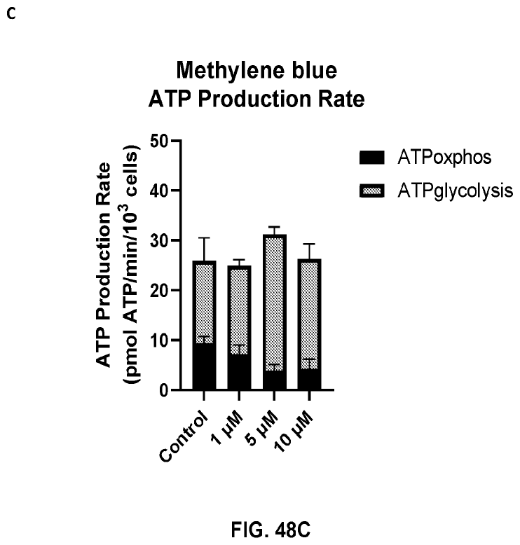

FIGS. 48A, 48B, and 48C show that treatment with methylene blue decreased oxygen consumption rates in GM28010 cells while maintain ATP production rates. There was a dose-dependent decrease in ATP production through OXPHOS and an increase in ATP production through glycolysis. Methylene Blue was used.

### Experimental Results

Treatment with the double combination of losmapimod and ginsenoside slightly increased respiration and ATP production rate through OXPHOS while decreasing ATP production through glycolysis.

Treatment with the double combination of ginsenoside and PF-06447475 slightly increased respiration and ATP production rate through OXPHOS while maintaining total ATP production rates.

Treatment with the double combination of losmapimod and PF-06447475 slightly increased respiration and ATP production rate through OXPHOS while maintaining total ATP production rates.

### Example 7

### Experiment 3 Fibroblast Cell Line GM28012 (Single-Compound Treatments)

GM28010 primary human fibroblasts obtained from patients with SURF1 Leigh syndrome were purchased from the Coriell Cell Repositories (Camden, N.J.; repository number GM28010).

GM00038 primary human fibroblasts purchased from the Coriell Cell Repositories (Camden, N.J., repository number GM00038) and were used as a healthy control.

Compounds used are shown in Table 4.

TABLE 4

| | Compound | Screen identified |
|---|---|---|
| 1 | Glabridin | TargetMol |
| 2 | Incyclinide | Pharmakon |
| 3 | Losmapimod | Personal communication |
| 4 | Miltefosine | TargetMol, Pharmakon, ReFrame |
| 5 | PF-06447475 | TargetMol |
| 6 | Posaconazole | TargetMol, Pharmakon, ReFrame |
| 7 | Sertaconazole | TargetMol, Pharmakon |
| 9 | cysteamine bitartrate | Personal communication |
| 10 | Poziotinib | TargetMol |
| 11 | Ginsenoside Z R1 | TargetMol |
| | (Zinglibroside) | |
| 12 | AM281 | TargetMol |

### Study Design—48 Hour Compound Treatment

### Cell Culture and Treatment

Fibroblasts were grown in glucose according to recommendation. Cells were plated in XF96 plates in culture medium and plated at 10,000 cells/well. The day after plating, the cells were treated with the compounds. The treatments completed in glucose medium and galactose medium. The compounds were tested at for 48 hours. The compounds were incubated at (37 C, 5% CO₂) until the day of the experiment. The fibroblasts were run at passage numbers: GM28012 SURF1 Fibroblasts P5.

### Seahorse Conditions

The assay medium was 10 mM glucose or galactose DMEM supplemented with 2 mM glutamine and 1 mM pyruvate. Injection Ports for basal glucose: Port A: 2 μM oligomycin; Port B: 2.5 μM FCCP; Port C: 4 μM FCCP; and Port D: 2 μM rotenone and antimycin A.

The data were normalized to cell number per well after the run. The Hoechst-stained nuclei were counted with a Operetta High-Content Imager.

### Experimental Results

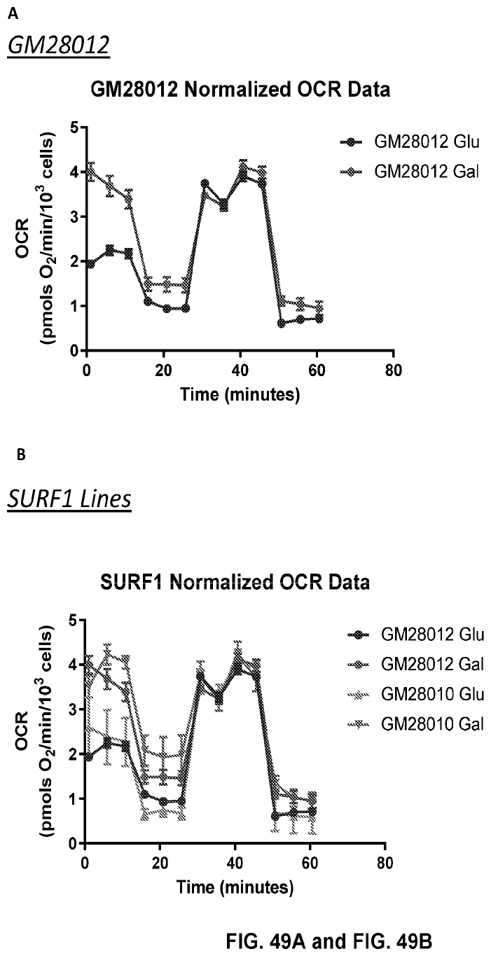

FIG. 49A and FIG. 49B show the compound treatment plate controls. FIG. 49A and FIG. 49B show that the GM28012 cells responded as expected in the Seahorse assay. Galactose culturing increased basal respiration, but an increase in maximal respiration was not seen in these experiments. Compared with the last experiment, GM28012 cells had oxygen consumption rates similar to those observed in GM28010 SURF1 fibroblasts.

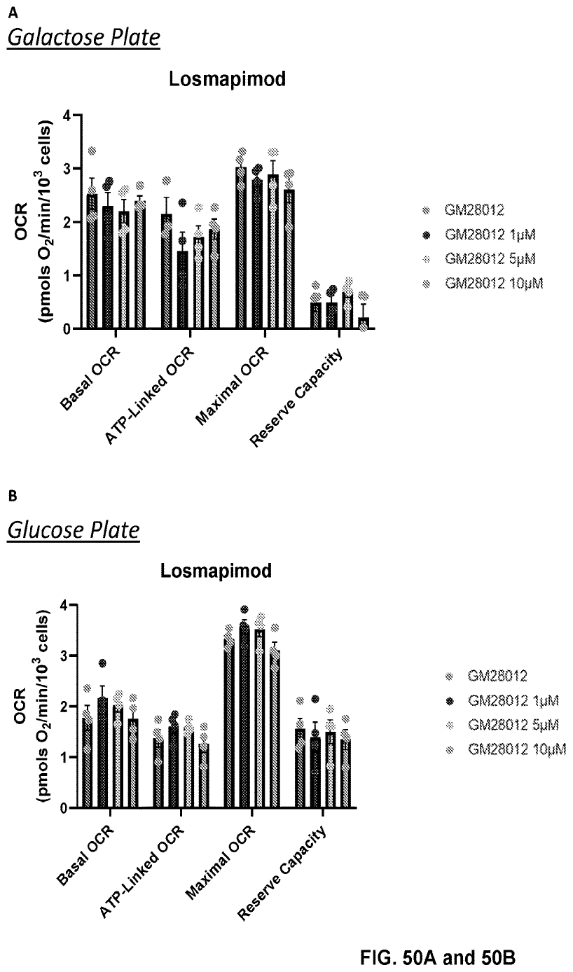

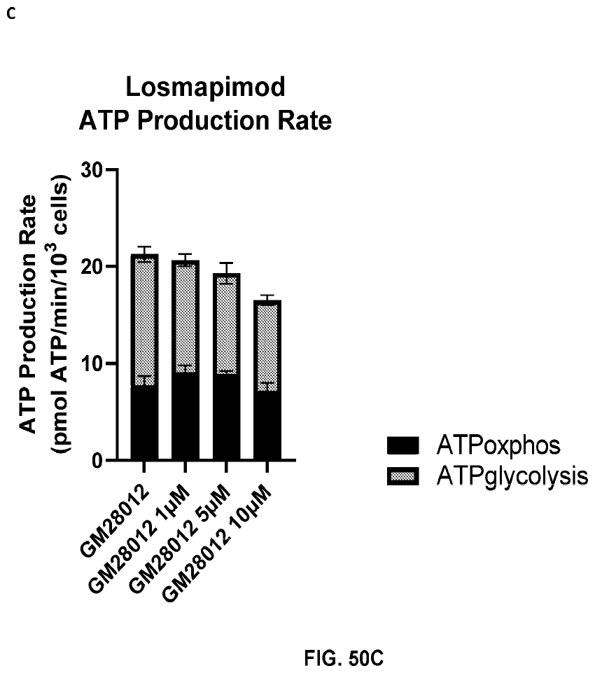

FIGS. 50A, 50B, and 50C show that treatment with losmapimod for 48 hours displayed increased in ATP-linked respiration in glucose-cultured cells. There was also a decrease in ATP production through glycolysis so that the total ATP production rate was maintained. Losmapimod did not increase respiration as much in these cells compared with GM28010 in the previous assay.

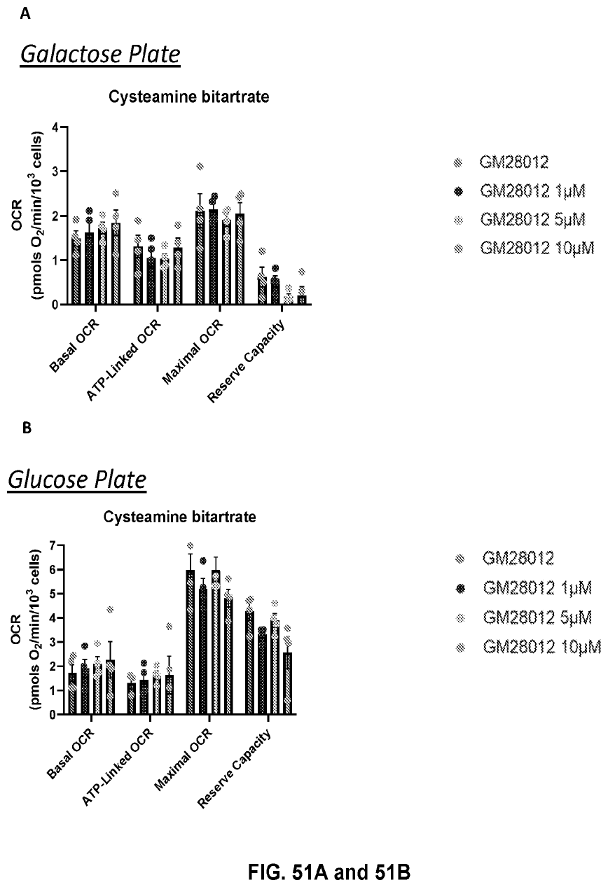

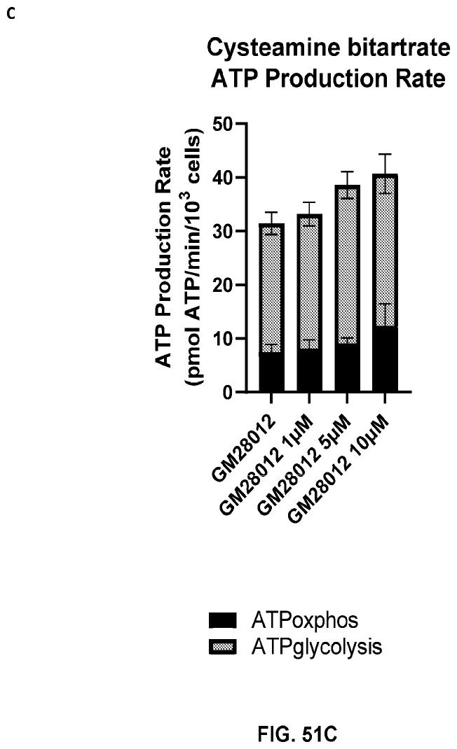

FIGS. 51A, 51B, and 51C show that treatment with cysteamine bitartrate for 48 hours increased basal respiration and ATP-Linked respiration in a dose-dependent manner in both glucose and galactose culture conditions decrease ATP production through glycolysis. However, this compound showed some toxicity in glucose-cultured cells with a dose-dependent decrease in maximal respiration and glycolysis.

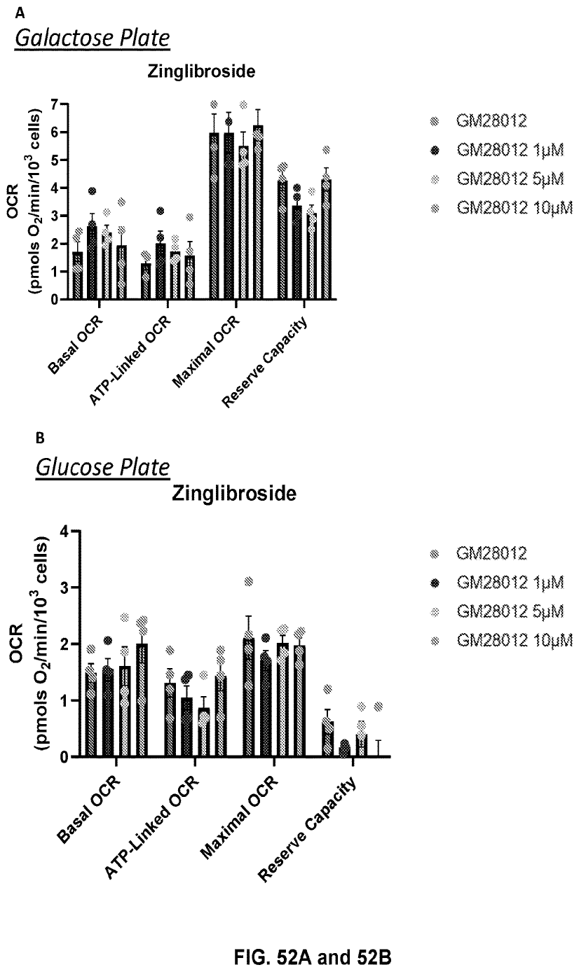

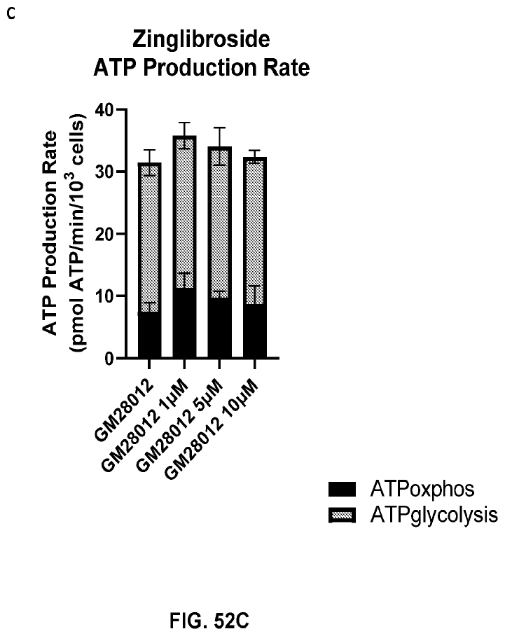

FIGS. 52A, 52B, and 52C show that treatment with Ginsenoside Z R1 for 48 hours did not result in changes to maximal respiration in either glucose or galactose conditions. There was an increase in ATP production through OXPHOS and glycolysis.

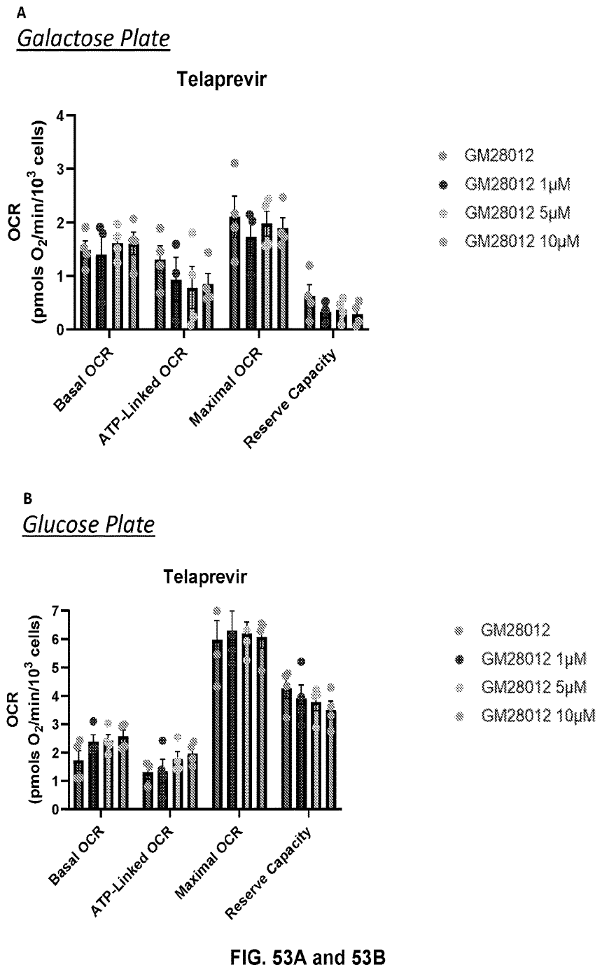

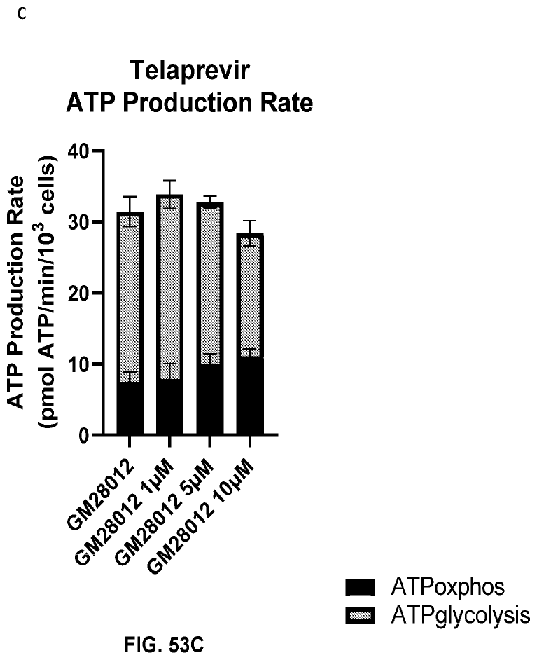

FIGS. 53A, 53B, and 53C show that treatment with telaprevir for 48 hours did not result in major changes to respiration in galactose conditions. In glucose, there was a dose-dependent increase in ATP-Linked respiration at ATP production through OXPHOS without any changes in maximal respiration.

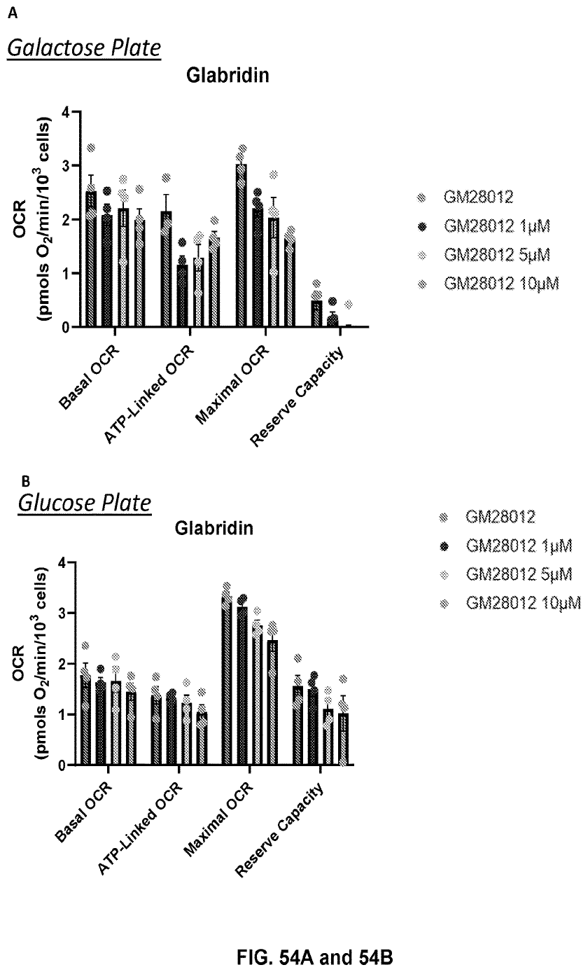

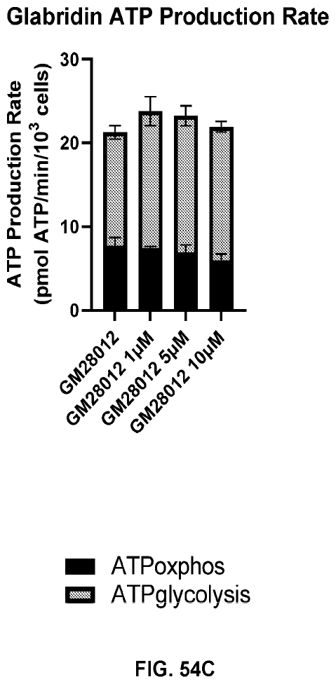

FIGS. 54A, 54B, and 54C show that treatment with glabridin for 48 hours decreased maximal respiration in both glucose or galactose conditions.

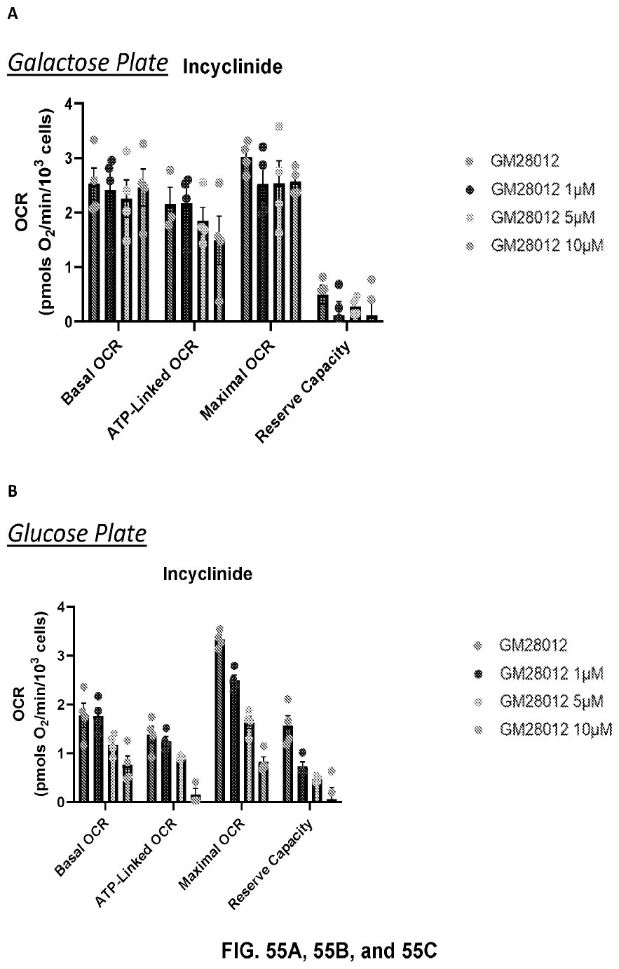

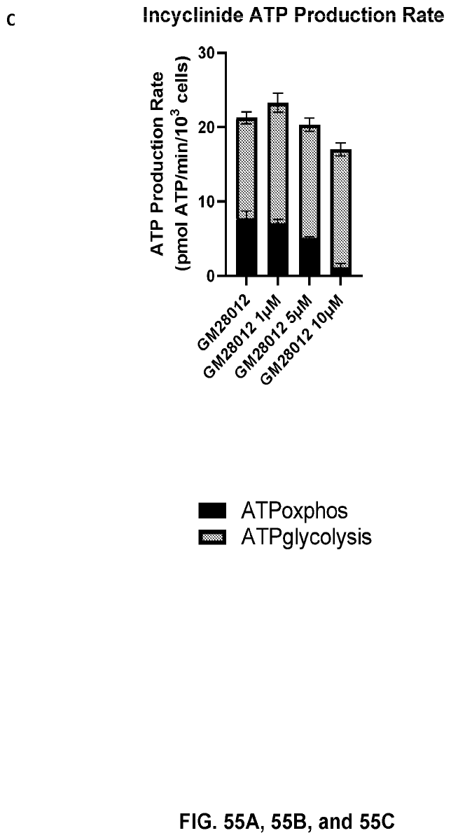

FIGS. 55A, 55B, and 55C show that treatment with incyclinide for 48 hours showed toxicity in glucose-cultured cells with a dose-dependent decrease in all respiratory parameters measured.

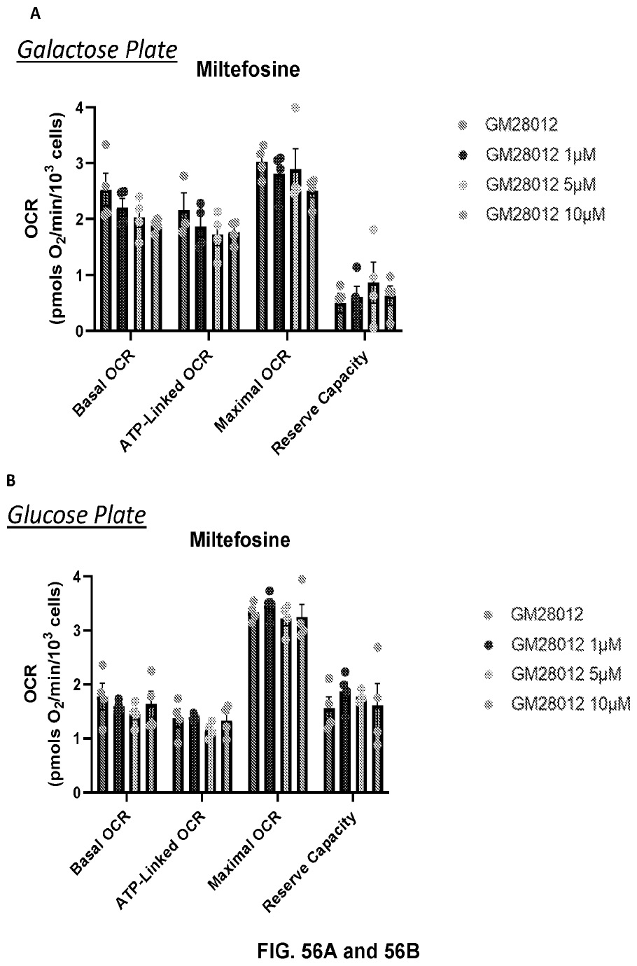

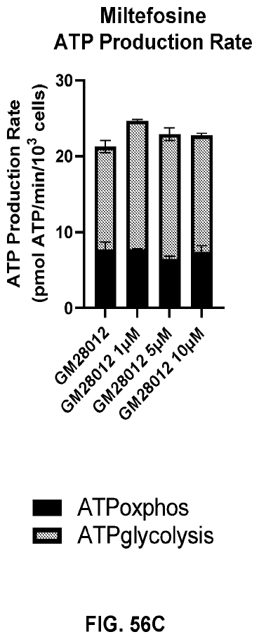

FIGS. 56A, 56B, and 56C show that treatment with miltefosine for 48 hours did not result in major changes to respiration in either glucose or galactose conditions. There was an increase in ATP production through glycolysis.

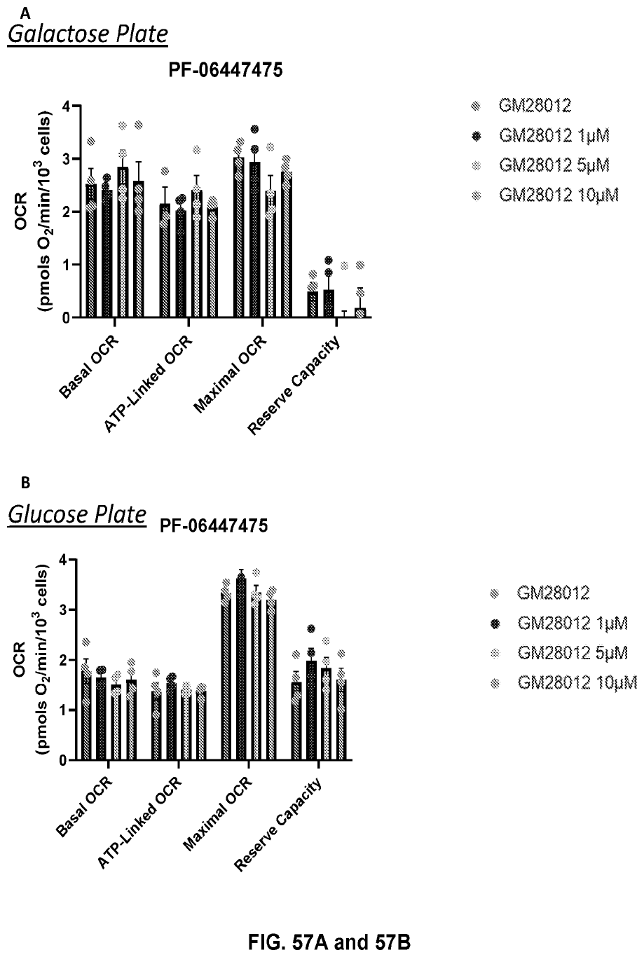

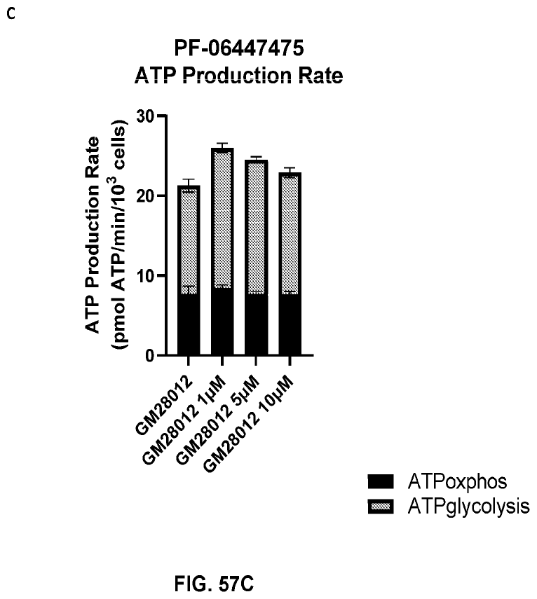

FIGS. 57A, 57B, and 57C show that treatment with PF-06447475 for 48 hours did not result increase maximal respiration in either glucose or galactose conditions. However, there was an increase in ATP production glycolysis with this compound that increased the total ATP Production rate at the 1 uM concentration.

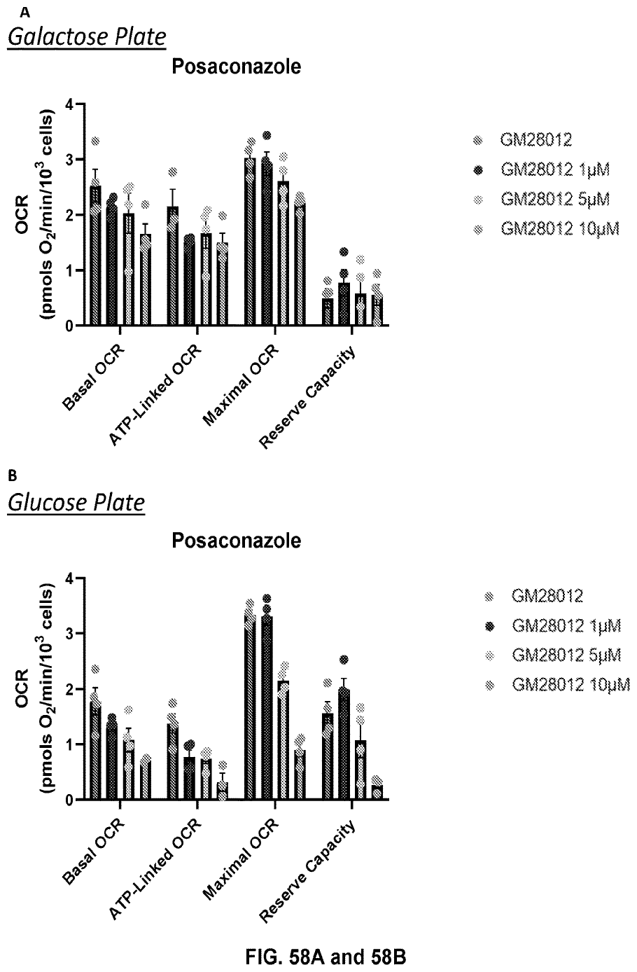

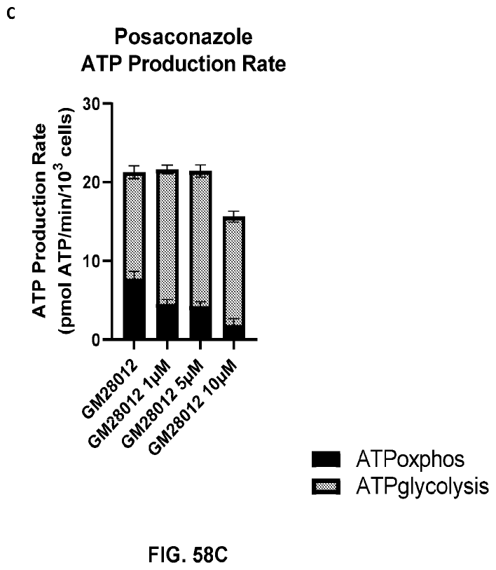

FIGS. 58A, 58B, and 58C show that treatment with posaconazole for 48 hours decreased respiration in both glucose or galactose conditions. The ATP production rate is compensation with increased glycolysis up until the highest concentration where total ATP drops.

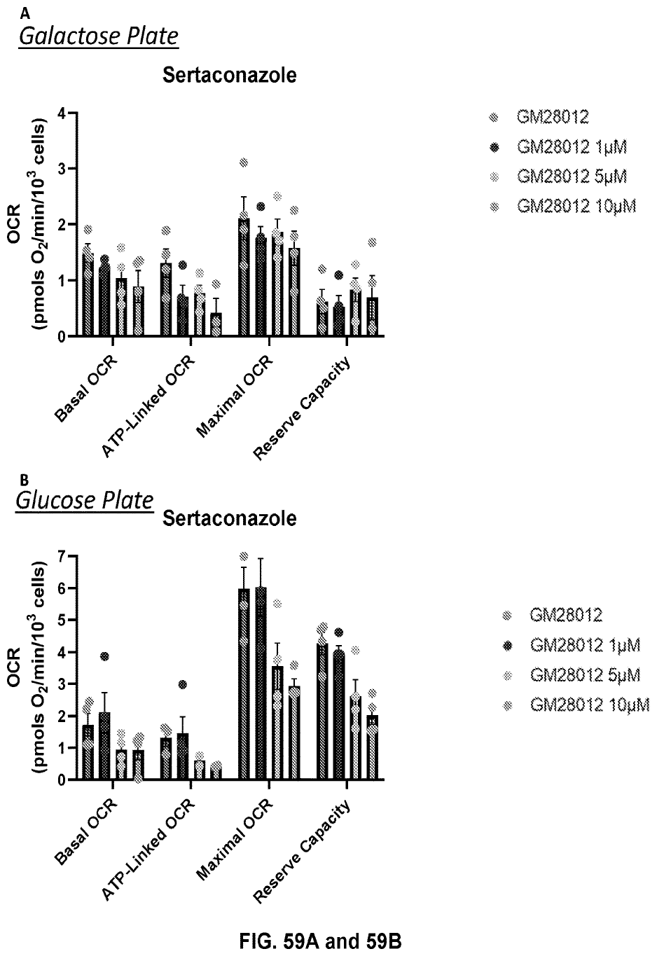

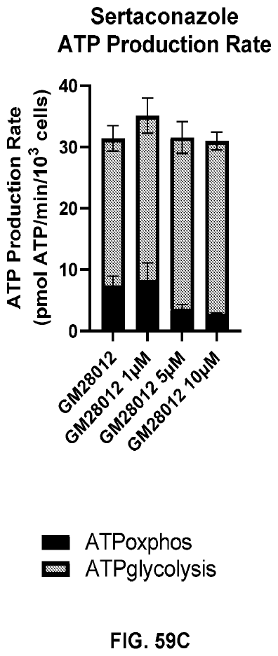

FIGS. 59A, 59B, and 59C show that treatment with Sertaconazole for 48 hours decreased respiration in both glucose and galactose and increased ATP production through glycolysis at higher concentrations.

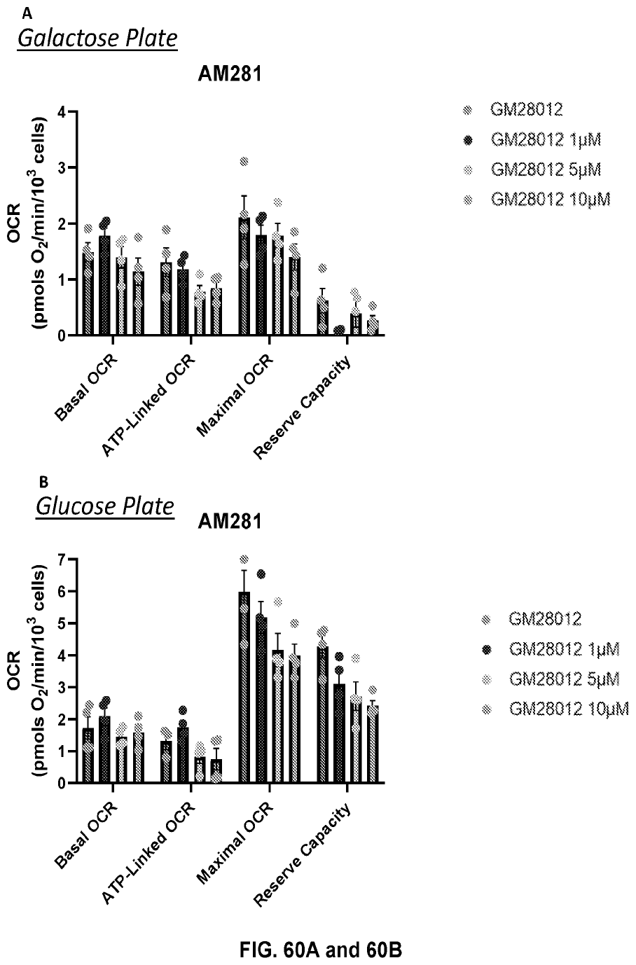

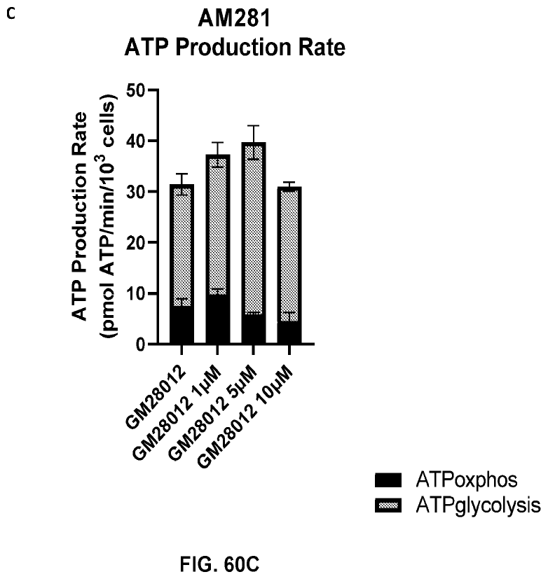

FIGS. 60A, 60B, and 60C show that treatment with AM281 for 48 hours increased ATP production rates but also showed a dose-dependent decreased maximal respiration.

### GM28012 Results Summary

Fibroblasts provided the characteristic response to injected compounds during the Seahorse assay. Glucose plate 2 displayed higher maximal respiration that glucose plate 1 cells.

Fibroblasts responded to galactose culturing for 48 hours by increasing basal respiration rates but not maximal respiration.

Experimental compounds did not always have the same respiratory effects in glucose and galactose cultured cells or comparing to GM28010, another SURF1 cell line previously tested.

None of the compounds tested increased maximal respiration. The compounds that showed the most interested results on respiration and total ATP production were:

### Losmapimod

Increased ATP-linked respiration and ATP production through OXPHOS and without changes in the total ATP production rate.
Effect of losmapimod was not as impressive as was observed in GM28010.

### Telaprevir

Dose-dependent increase in ATP production through OXPHOS and without changes in the total ATP production rate.

### Cysteamine Bitartrate

Dose-dependent increase in ATP production through OXPHOS and total ATP production rate.
Ginsenoside Z R1
1 μM concentration increased ATP production through OXPHOS and total ATP production rate.

### Example 8

### Comparison of Experiment 1 and Experiment 3

A comparison of the Seahorse assay in two fibroblast lines is shown in Table 5.

TABLE 5

| Compound | Will's Fibroblast GM28010 | Delta-like Fibroblast GM28012 |
|---|---|---|
| Posacanazole | ↓ respiration; toxic at all conc | ↓ respiration, total ATP rate |
| | | compensated by glycolysis |
| | | until highest concentration |
| | | (10 uM) where ATP drops |
| Sertaconazole | ↓ maximal OCR@ all conc; | Maximal OCR maintained at |
| | no effect on ATP production | lower concentration; ↑ATP |
| | | via glycolysis at higher conc. |
| Glabridin | No effect on respiration in | ↓ maximal OCR in both |
| | both conditions | conditions |
| Zinglibroside | No effect on respiration in | No effect on respiration in |
| | both conditions: ↑ATP via | both conditions; ↑ATP via |
| | glycolysis and OXPHOS @ | glycolysis and OXPHOS @ |
| | 10 μM | all conc |
| AM281 | No effect on respiration in | dose-dependent ↓ maximal |
| | both conditions; ↑ATP via | OCR. ↑ATP via glycolysis @ |
| | glycolysis @5 and 10 μM | 1 μM and 5 μM |
| Cysteamine | No effect on respiration in | ↑basal respiration and ATP- |
| Bitrartate | both conditions; ↓ATP via | Linked respiration in a dose- |
| | glycolysis @ 10 μM | dependent manner in both |
| | | conditions. ↓ATP production |
| | | through glycolysis |
| PF-06447475 | No effect on respiration in | No effect on respiration in |
| | both conditions. ↑ ATP | both conditions. ↑ ATP |
| | production through both | production through |
| | OXPHOS and glycolysis. | glycolysis. At 1 μM |
| Losmapimod | 12-15% increases in | ↑ATP-linked respiration in |
| | respiration in ATP-linked and | glucose-cultured cells. ↓ATP |
| | maximal respiration in both | production via glycolysis, |
| | conditions. ↓ ATP production | total ATP production rate |
| | through glycolysis. | maintained. % ↑lower than vs |
| | | Will's fibroblasts. |
| Telepravir | No effect on respiration in | No effect on respiration in |
| | both conditions | galactose. Glucose: dose- |
| | | dependent ↑ATP-Linked |
| | | respiration at ATP production |
| | | through OXPHOS, no change |
| | | in maximal respiration. |
| Miltefosine | No effect on respiration in | No effect on respiration in |
| | both conditions | both conditions; ↑ in ATP |
| | | production through |
| | | glycolysis. |
| Incyclinide | ↑ basal respiration and ATP- | Toxicity observed in glucose |
| | linked respiration in | media |
| | galactose-cultured cells. | |
| | Toxicity in glucose | |
| Poziotinib | No effect on respiration in | Not tested |
| | both conditions; ↑ATP via | |
| | glycolysis and toxicity at | |
| | higher concentration | |

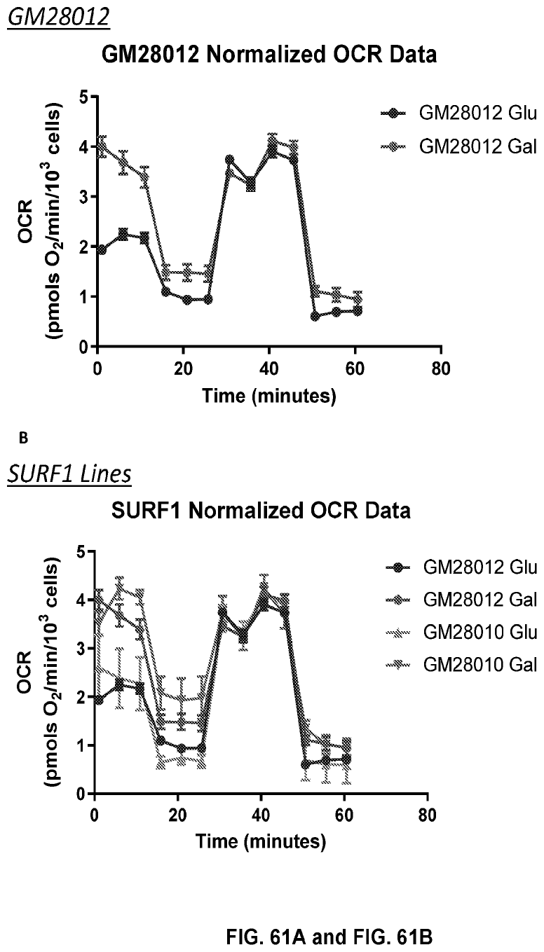

FIG. 61A and FIG. 61B show compound treatment controls. FIG. 61A and FIG. 61B show that the GM28012 cells responded as expected in the Seahorse assay. Galactose culturing increased basal respiration, but an increase in maximal respiration was not seen in these experiments. Compared with the last experiment, GM28012 cells had oxygen consumption rates similar to what was observed in GM28010 SURF1 fibroblasts.

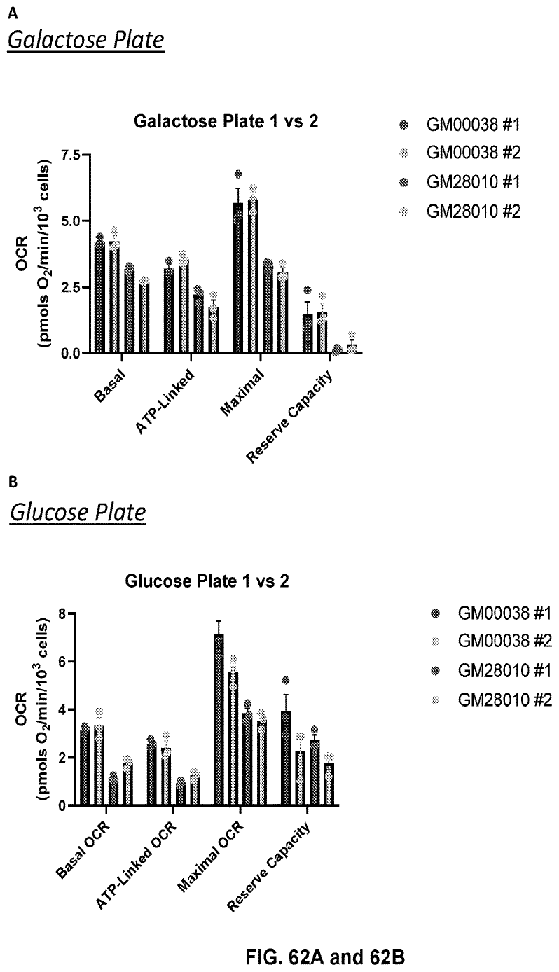

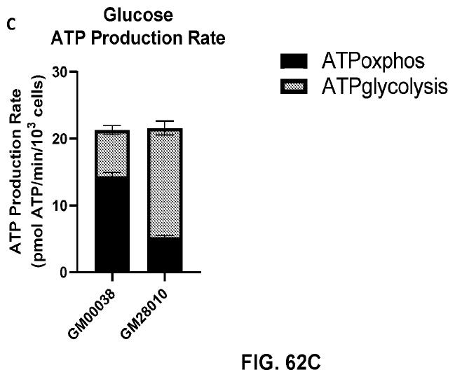

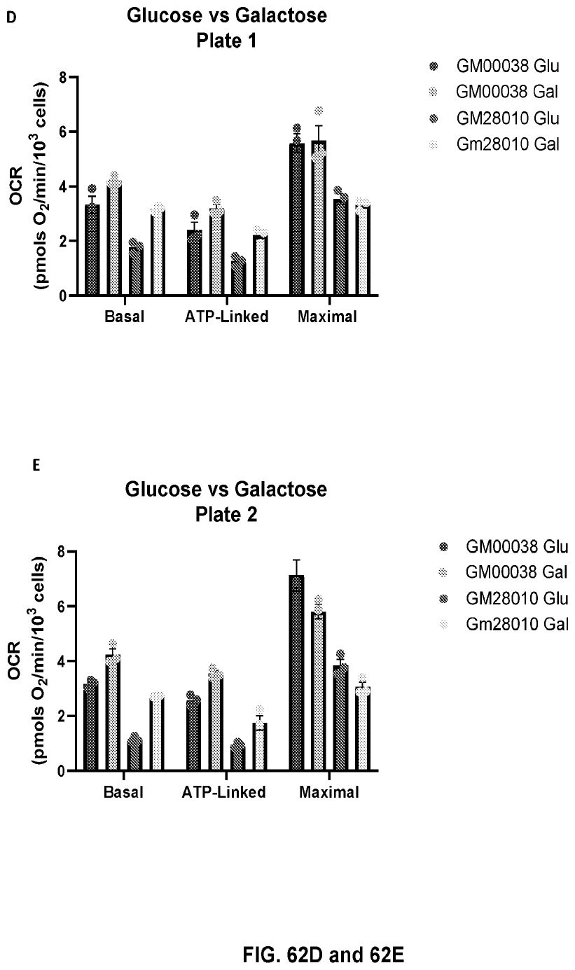

FIGS. 62A, 62B, 62C, 62D, and 62E show the controls on the compound treatment plates. FIGS. 62A, 62B, 62C, 62D, and 62E show that the SURF1 mutant cells display a severe respiratory defect compared to a control cell line (GM00038F). There is a decrease in all respiratory parameters and an increase in ATP production through glycolysis to compensate for the respiratory defect. The cells responded similarly on the four different assay plates (2 glucose plates and 2 galactose plates). However, the control line had a slightly lower response to FCCP and, therefore, maximal respiration on plate #2 in the glucose-culture cells. In these cells galactose culturing increased basal respiration, but an increase in maximal respiration was not seen in these experiments.

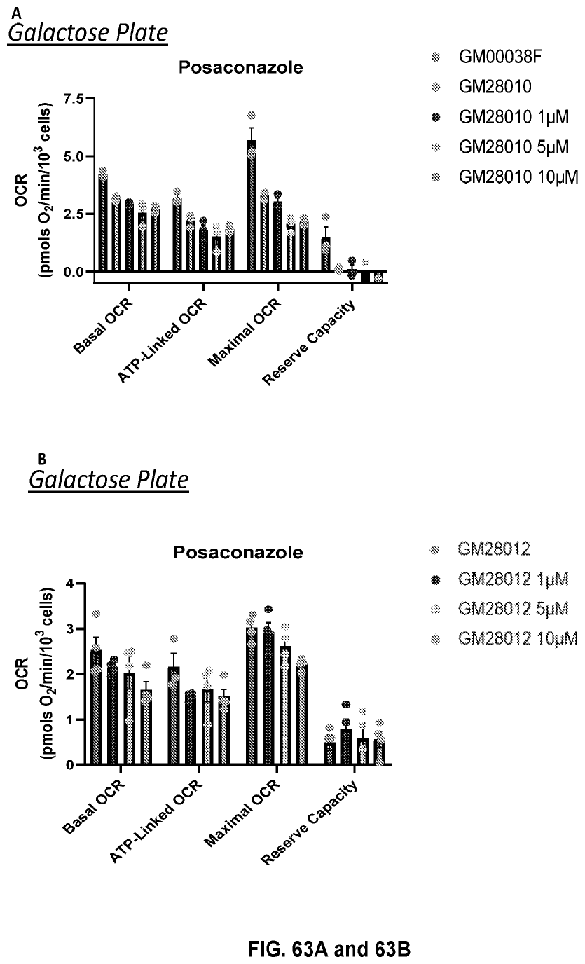

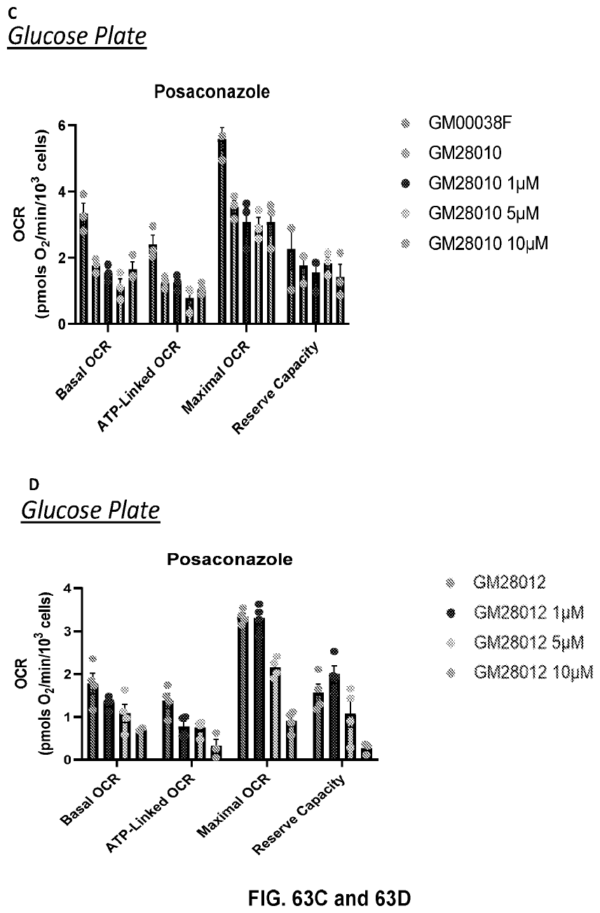

FIGS. 63A, 63B, 63C, 63D, 63E, and 63F show that treatment with posaconazole for 48 hours did not result in major changes to respiration in either glucose or galactose conditions. At higher concentrations, it decreased respiration. Treatment with posaconazole for 48 hours decreased respiration in both glucose or galactose conditions. The ATP production rate is compensation with increased glycolysis up until the highest concentration where total ATP drops.

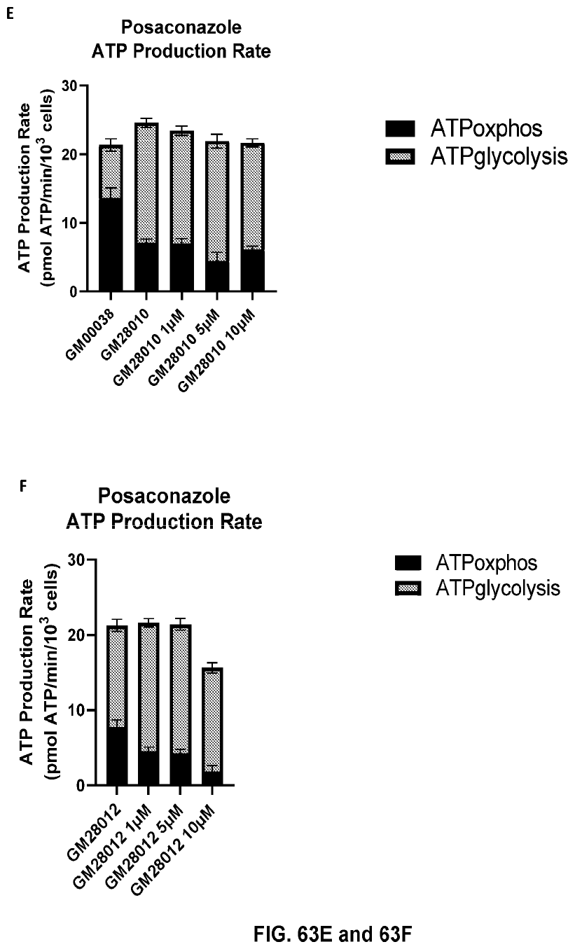

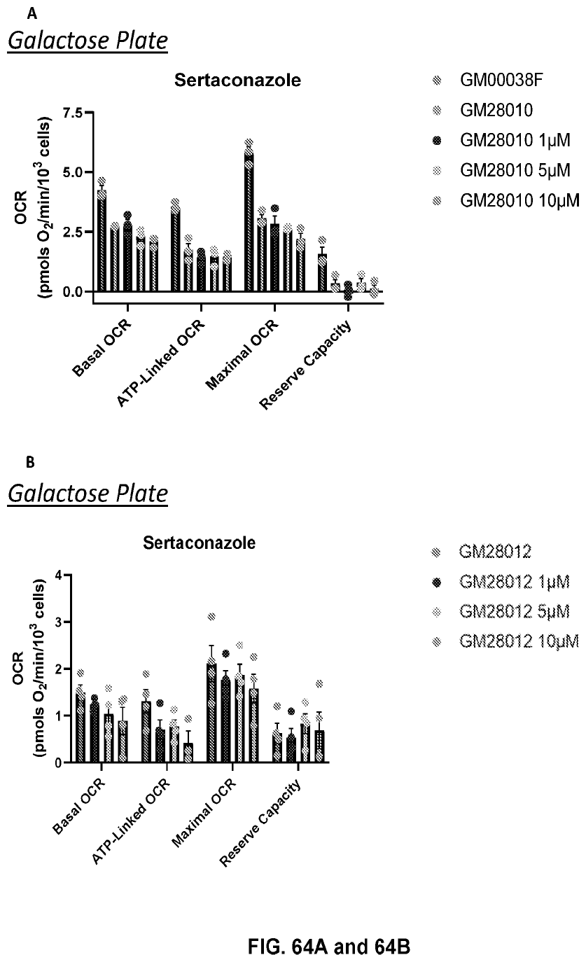

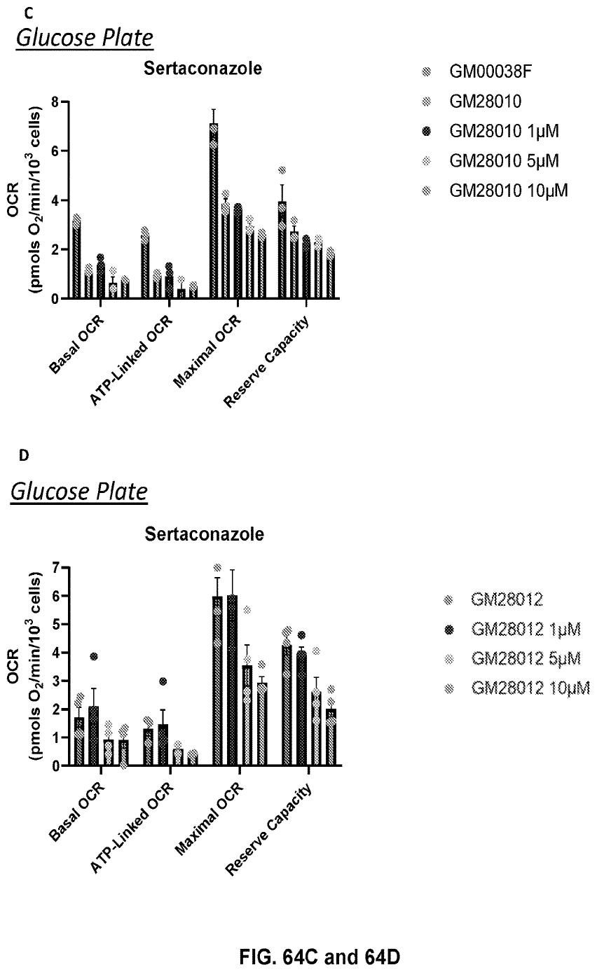

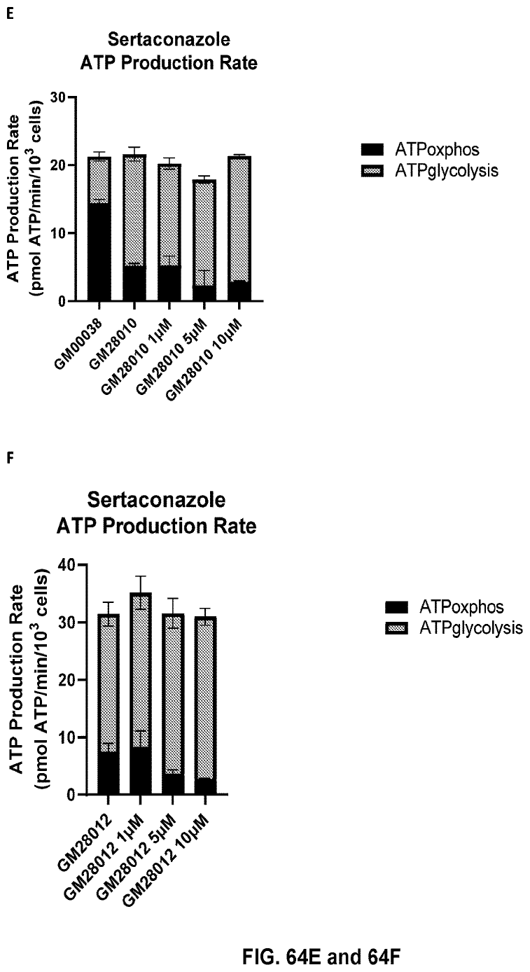

FIGS. 64A, 64B, 64C, 64D, 64E, and 64F show that treatment with Sertaconazole for 48 hours did not result in major changes to respiration in either glucose or galactose conditions. Treatment with Sertaconazole for 48 hours decreased respiration in both glucose and galactose and increased ATP production through glycolysis at higher concentrations.

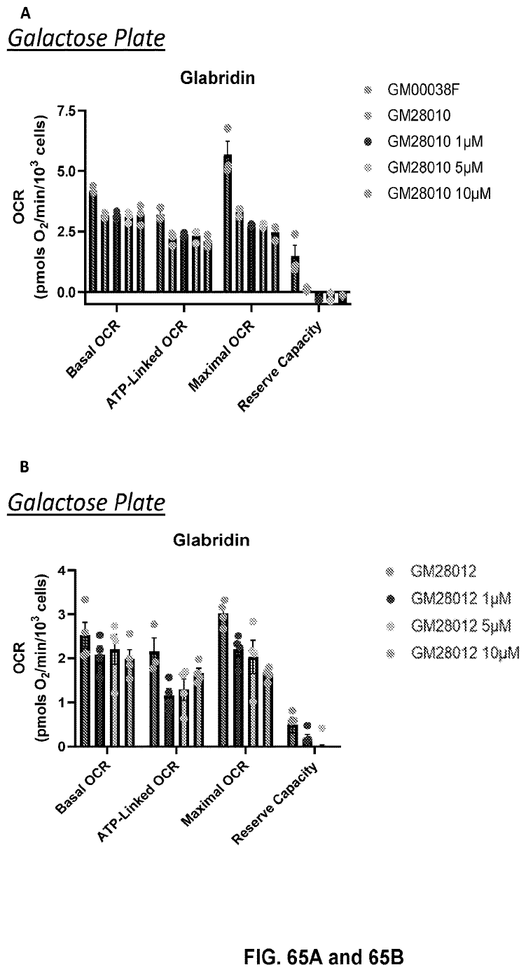

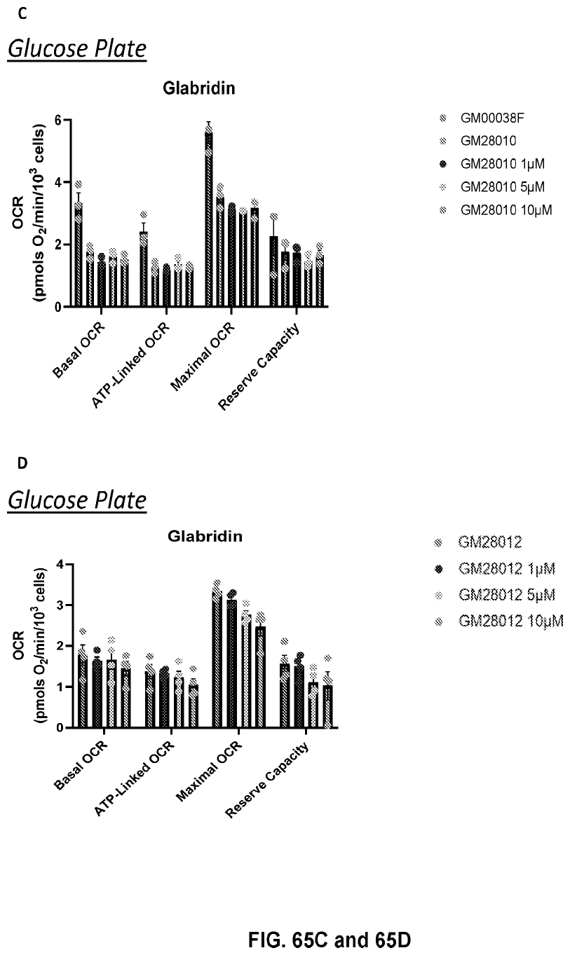

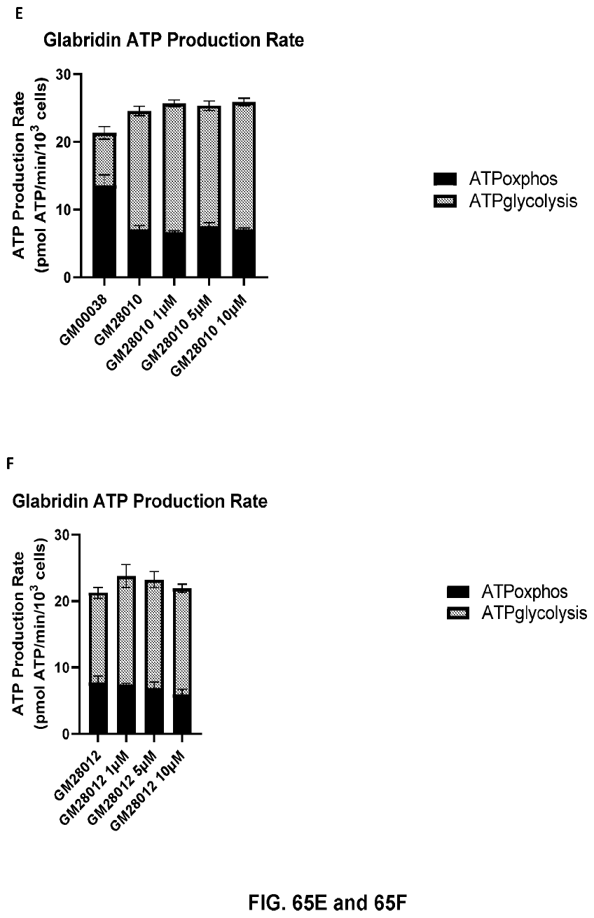

FIGS. 65A, 65B, 65C, 65D, 65E, and 65F show that treatment with glabridin for 48 hours did not result in major changes to respiration in either glucose or galactose conditions. Treatment with glabridin for 48 hours decreased maximal respiration in both glucose or galactose conditions

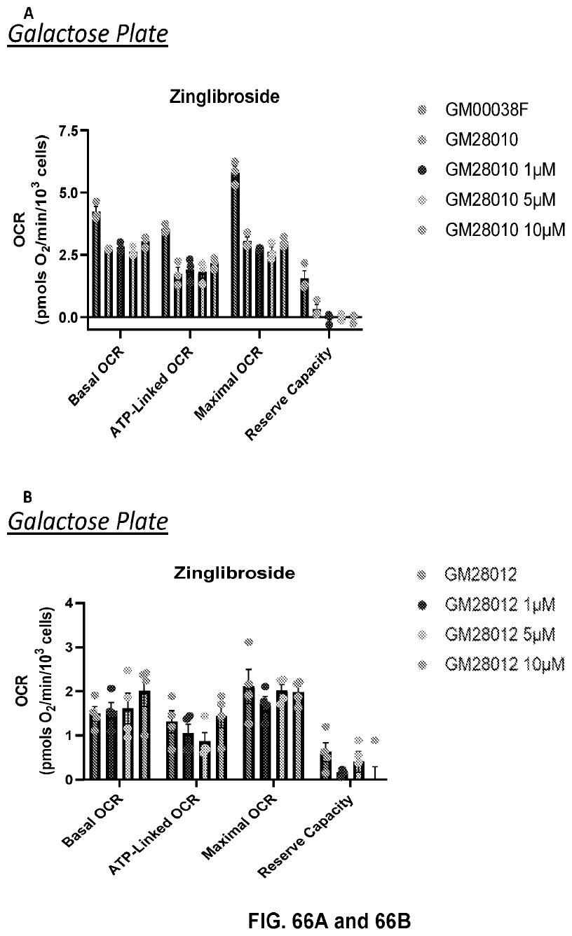

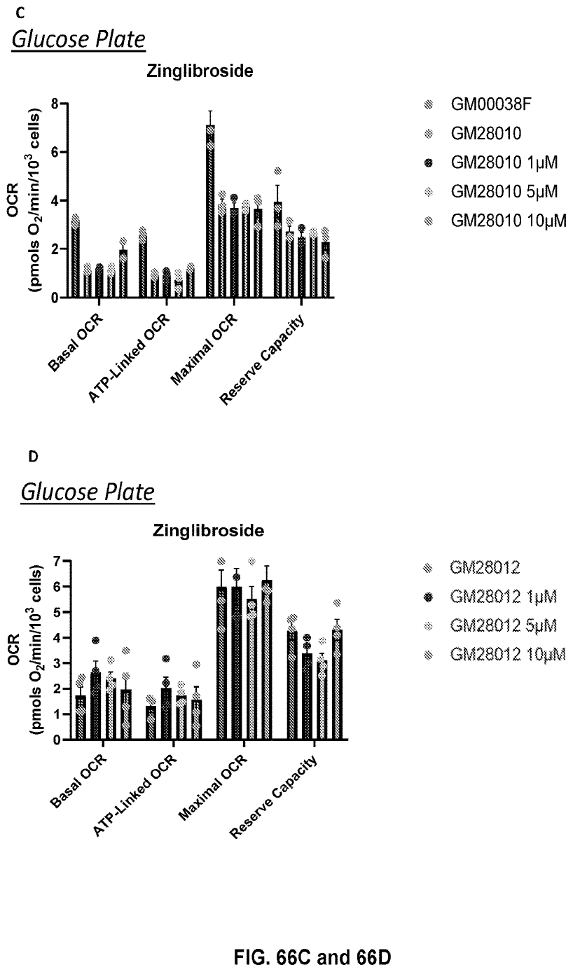

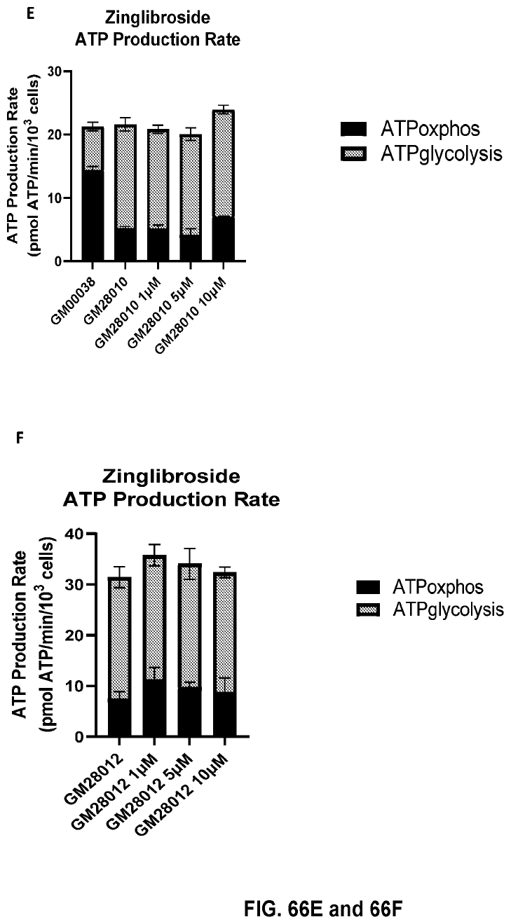

FIGS. 66A, 66B, 66C, 66D, 66E, and 66F show that treatment with zinglibroside for 48 hours did not result in changes to maximal respiration in either glucose or galactose conditions. There was an increase in ATP production through OXPHOS and glycolysis with the 10 μM concentration. Treatment with zinglibroside for 48 hours did not result in changes to maximal respiration in either glucose or galactose conditions. There was an increase in ATP production through OXPHOS and glycolysis.

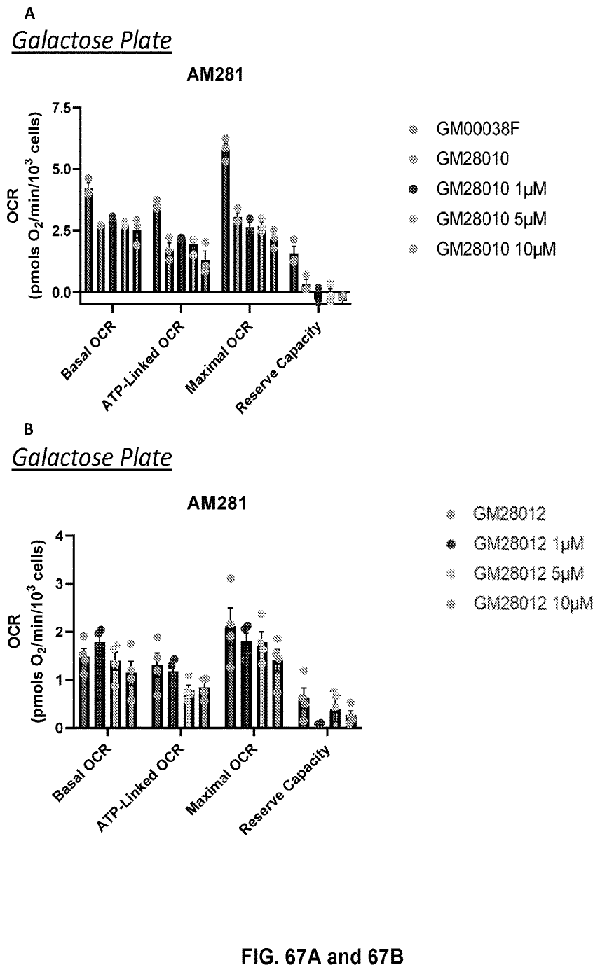

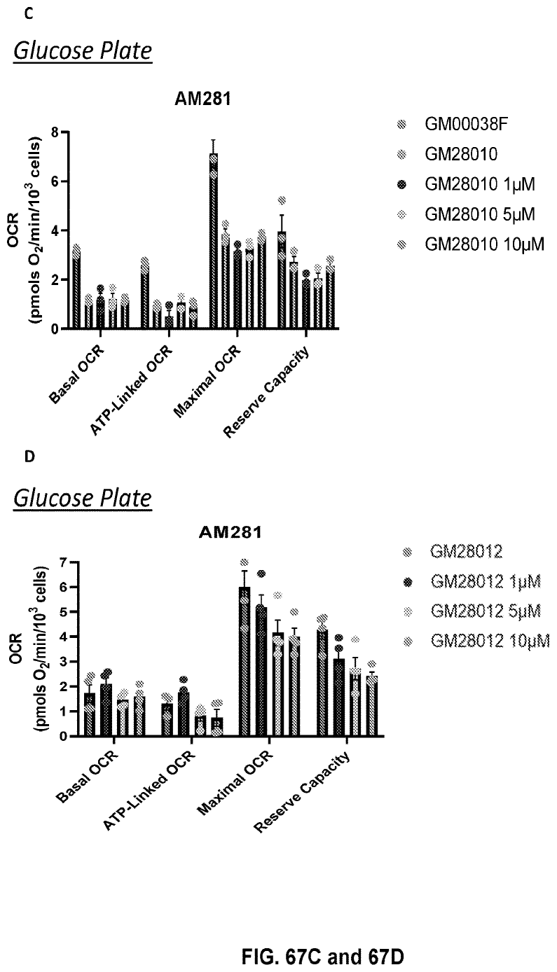

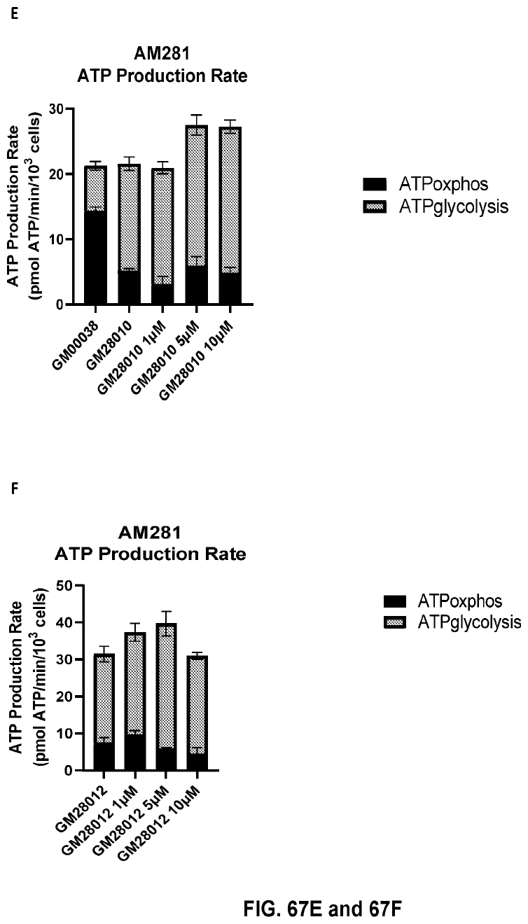

FIGS. 67A, 67B, 67C, 67D, 67E, and 67F show that treatment with AM281 for 48 hours did not result in major changes to respiration in either glucose or galactose conditions. There was an increase in ATP production primarily through glycolysis at the 5 and 10 μM concentrations. Treatment with AM281 for 48 hours increased ATP production rates but also showed a dose-dependent decreased maximal respiration.

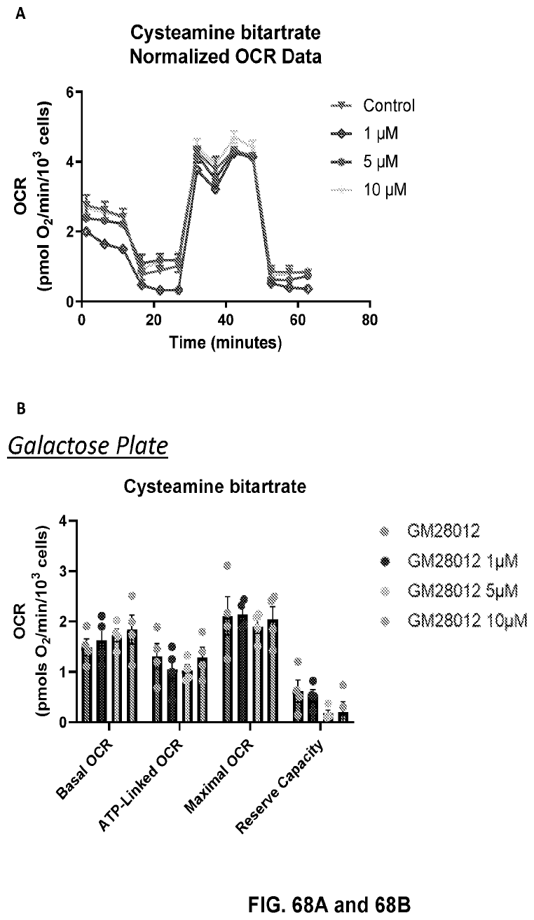

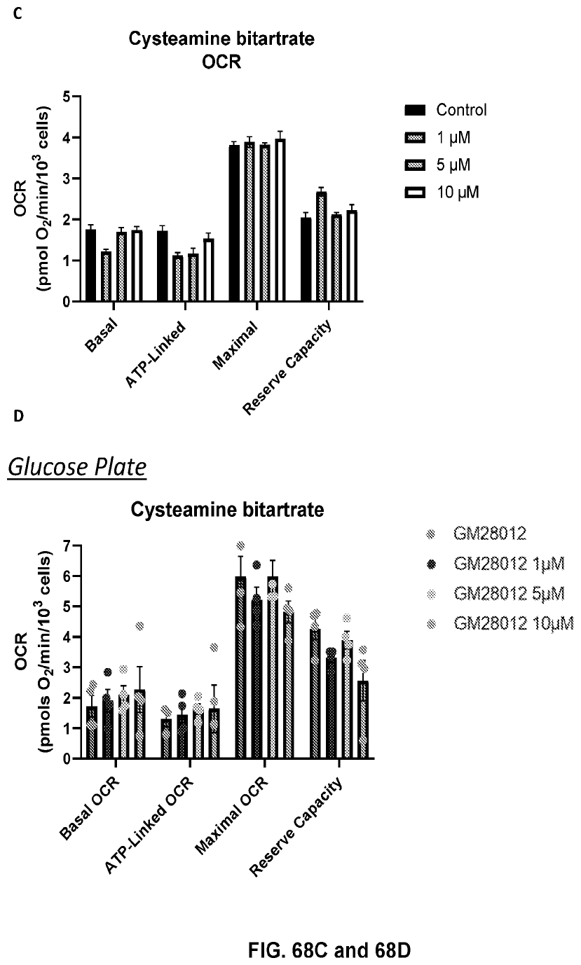

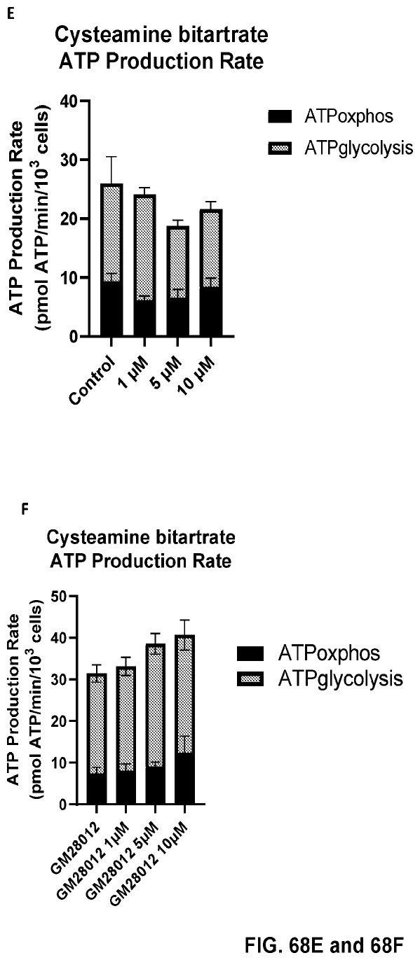

FIGS. 68A, 68B, 68C, 68D, 68E, and 68F show that treatment with cysteamine bitartrate did not alter oxygen consumption rates in GM28010 cells. ATP production rate through glycolysis was decreased with the higher concentrations of cysteamine bitartrate. Treatment with cysteamine bitartrate for 48 hours increased basal respiration and ATP-Linked respiration in a dose-dependent manner in both glucose and galactose culture conditions, decrease ATP production through glycolysis. However, this compound showed some toxicity in glucose-cultured cells with a dose-dependent decrease in maximal respiration and glycolysis.

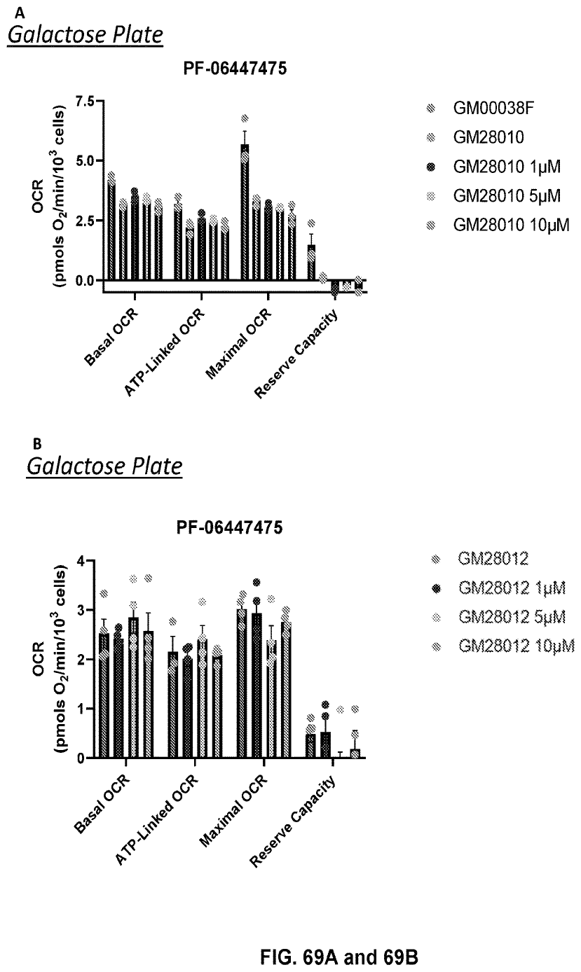

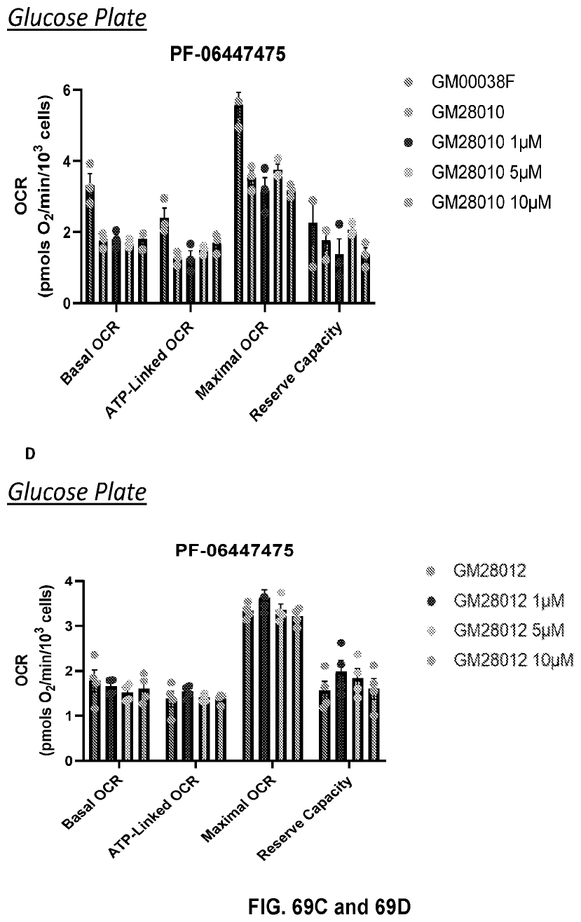

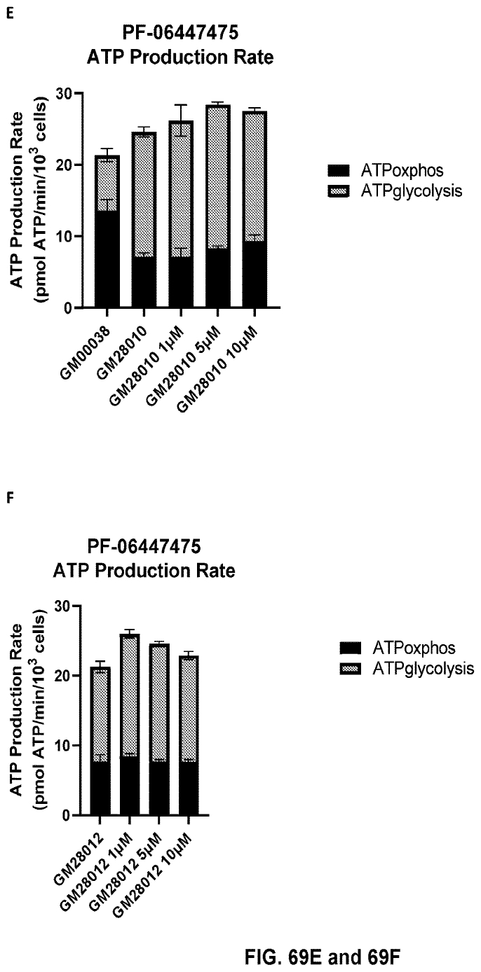

FIGS. 69A, 69B, 69C, 69D, 69E, and 69F show that treatment with PF-06447475 for 48 hours did not result increase maximal respiration in either glucose or galactose conditions. However, there was an increase in ATP production through both OXPHOS and glycolysis with this compound. Treatment with PF-06447475 for 48 hours did not result increase maximal respiration in either glucose or galactose conditions. However, there was an increase in ATP production glycolysis with this compound that increased the total ATP Production rate at the 1 uM concentration.

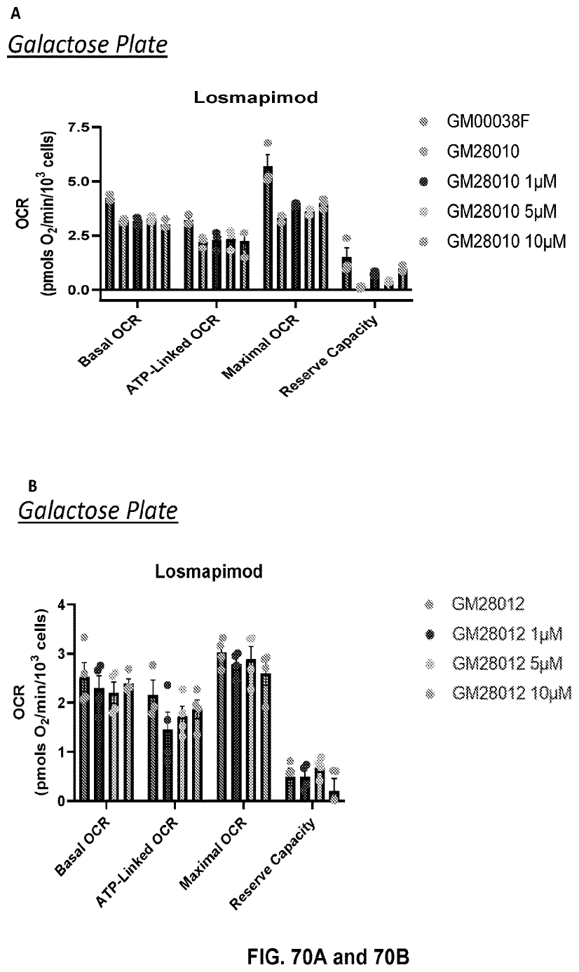

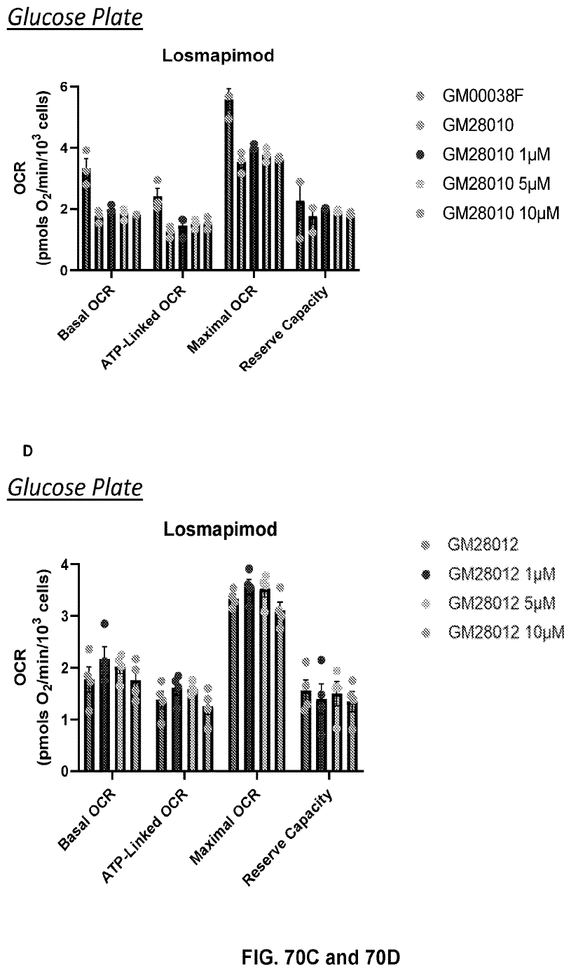

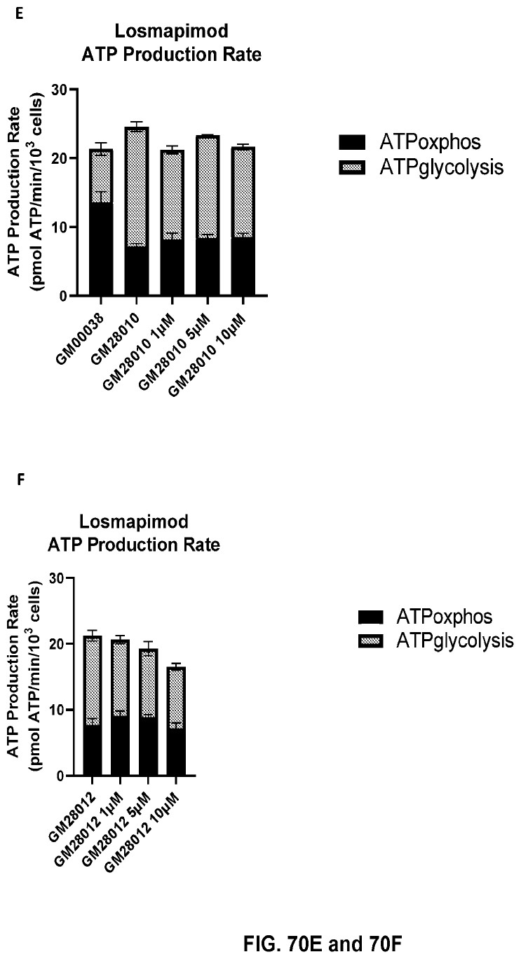

FIGS. 70A, 70B, 70C, 70D, 70E, and 70F show that treatment with losmapimod for 48 hours displayed 12-15% increases in respiration in ATP-linked and maximal respiration in both glucose and galactose-cultured cells. There was also a decrease in ATP production through glycolysis. Follow up with longer compound treatments or an expanded dose-response could be performed. Treatment with losmapimod for 48 hours displayed increased in ATP-linked respiration in glucose-cultured cells. There was also a decrease in ATP production through glycolysis so that the total ATP production rate was maintained. Losmapimod did not increase respiration as much in these cells compared with GM28010 in the previous assay.

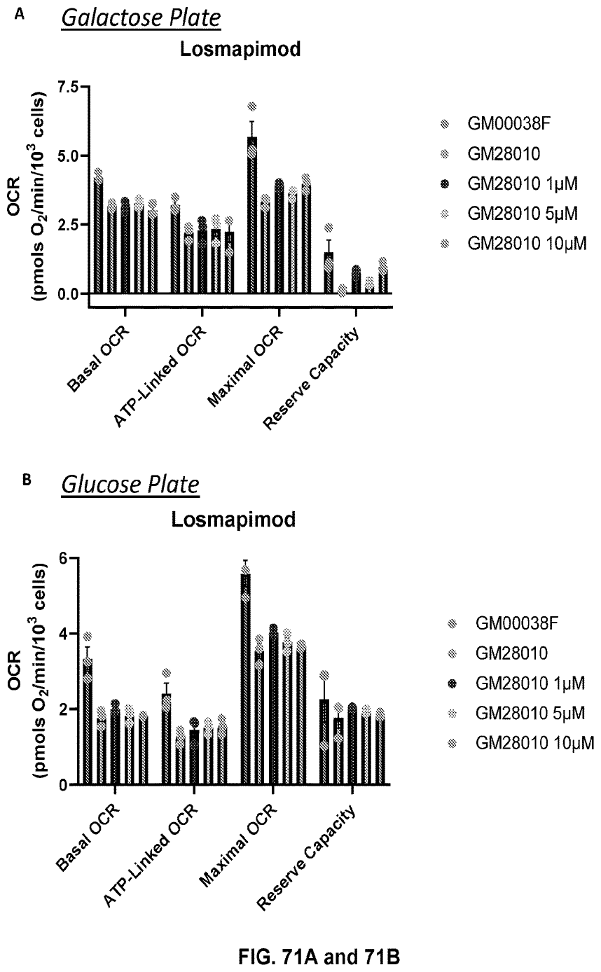

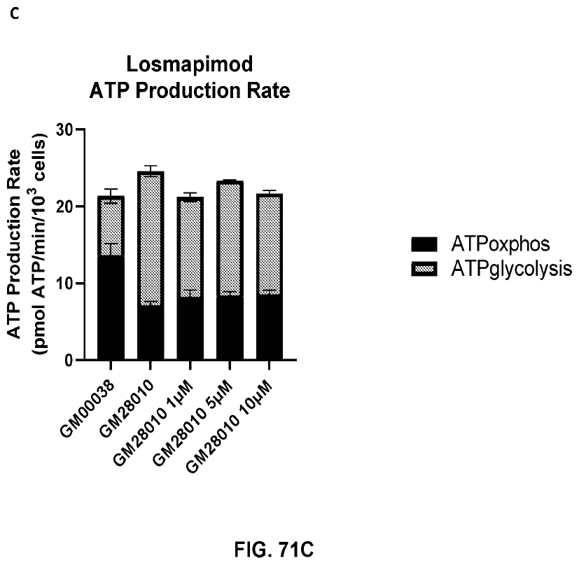

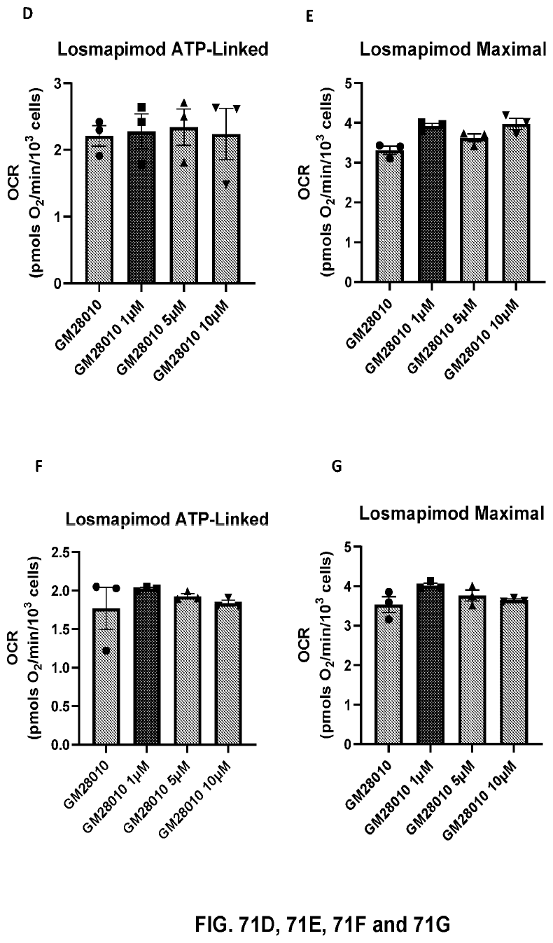

FIGS. 71A, 71B, 71C, 71D, 71E, 71F, and 71G show that treatment with losmapimod for 48 hours displayed 12-15% increases in respiration in ATP-linked and maximal respiration in both glucose and galactose-cultured cells. There was also a decrease in ATP production through glycolysis. Experiments with longer compound treatments or an expanded dose-response could be performed.

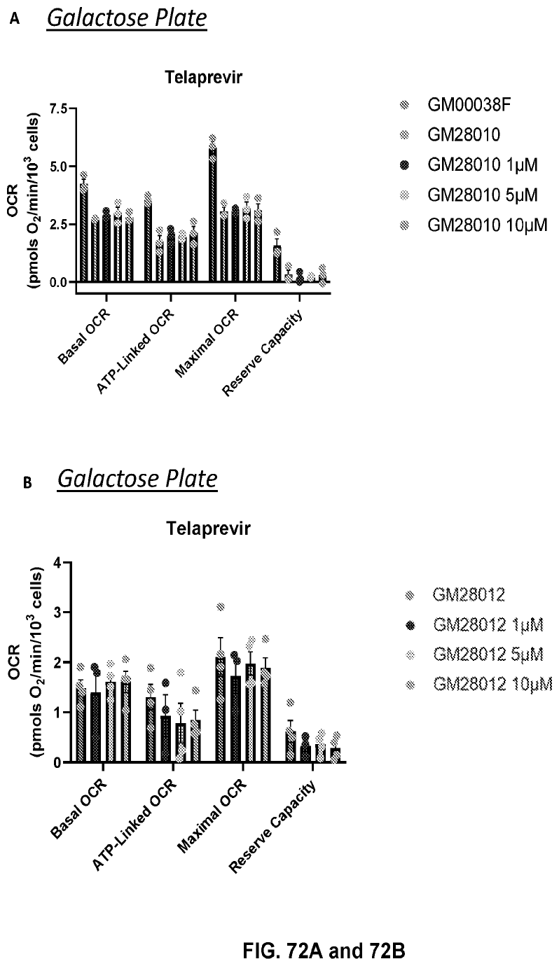

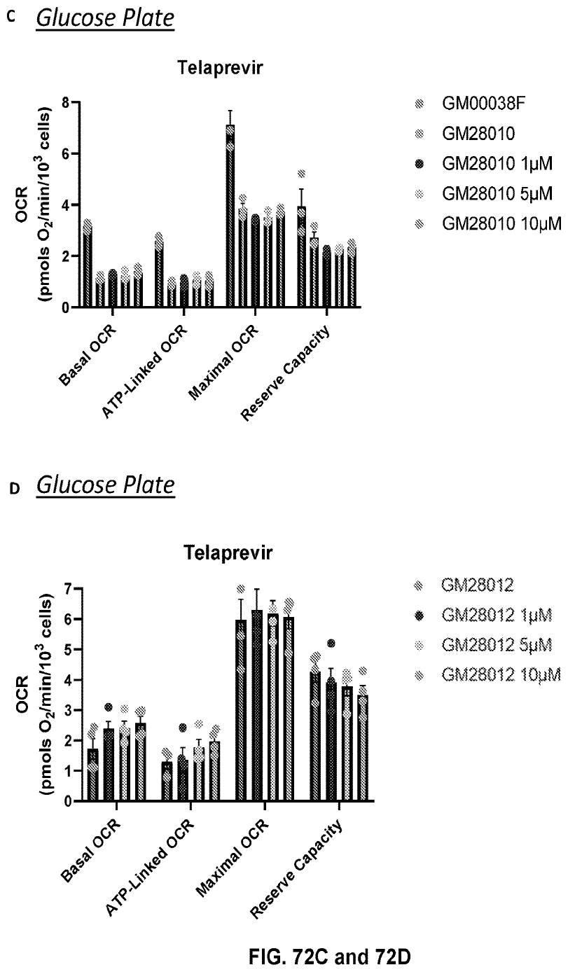

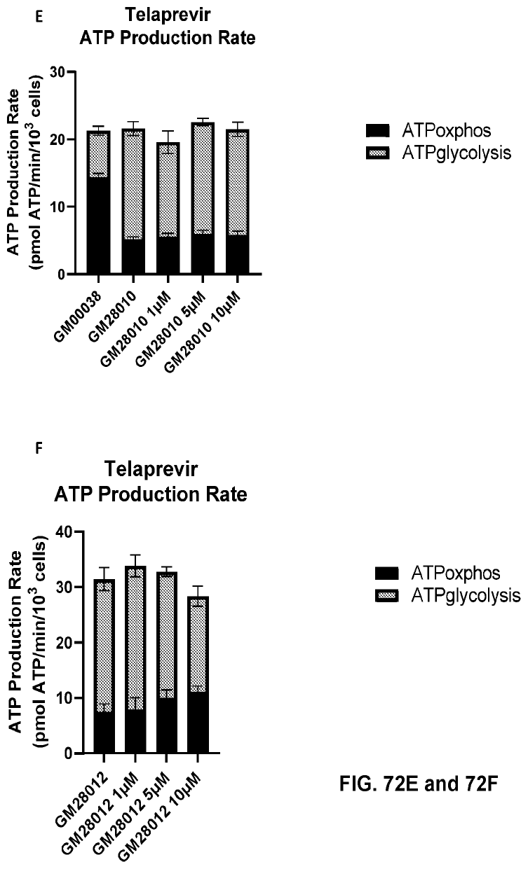

FIGS. 72A, 72B, 72C, 72D, 72E, and 72F show that treatment with telaprevir for 48 hours did not result in major changes to respiration in either glucose or galactose conditions. Treatment with telaprevir for 48 hours did not result in major changes to respiration in galactose conditions. In glucose, there was a dose-dependent increase in ATP-Linked respiration at ATP production through OXPHOS without any changes in maximal respiration.

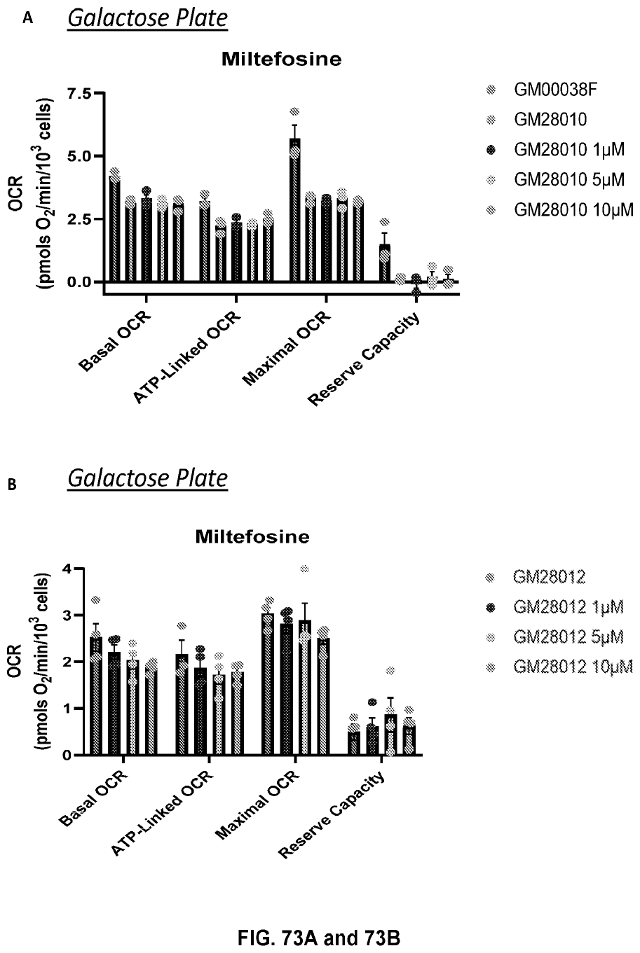

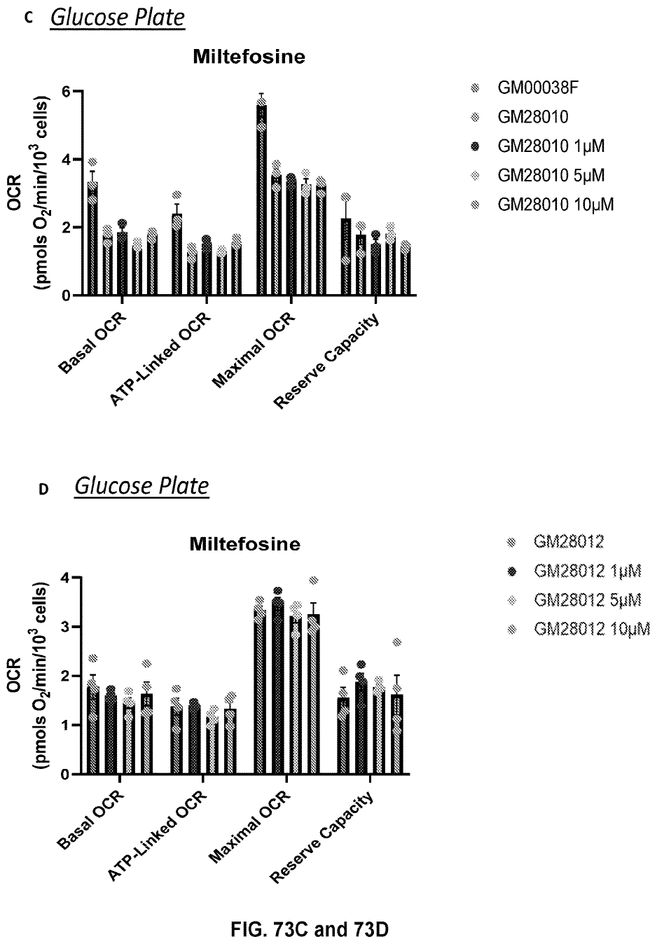

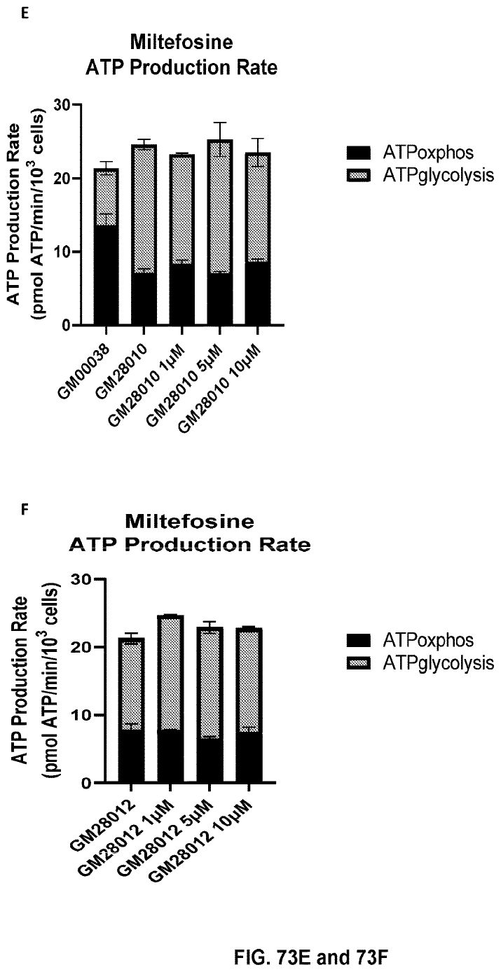

FIGS. 73A, 73B, 73C, 73D, 73E, and 73F show that treatment with miltefosine for 48 hours did not result in major changes to respiration in either glucose or galactose conditions. Treatment with miltefosine for 48 hours did not result in major changes to respiration in either glucose or galactose conditions. There was an increase in ATP production through glycolysis.

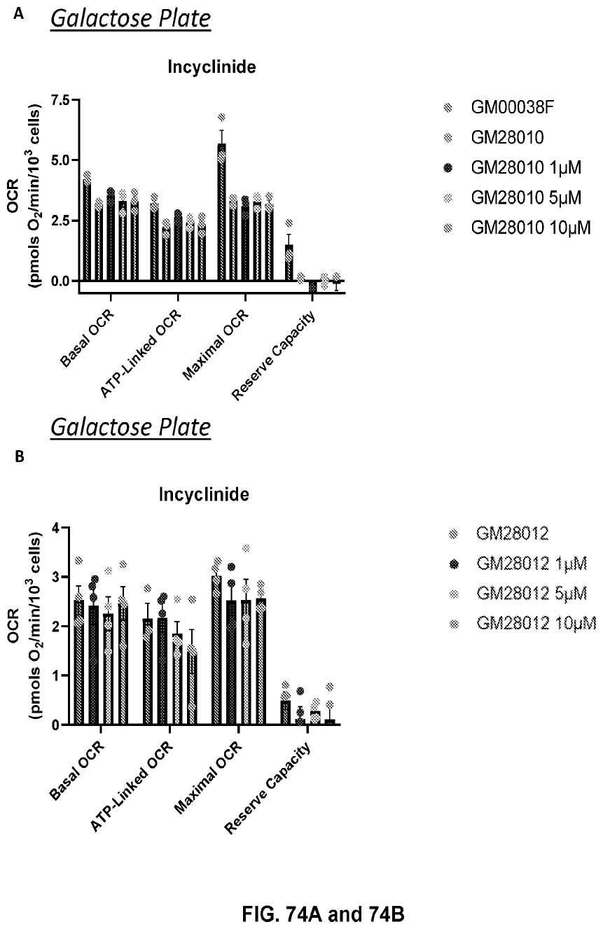

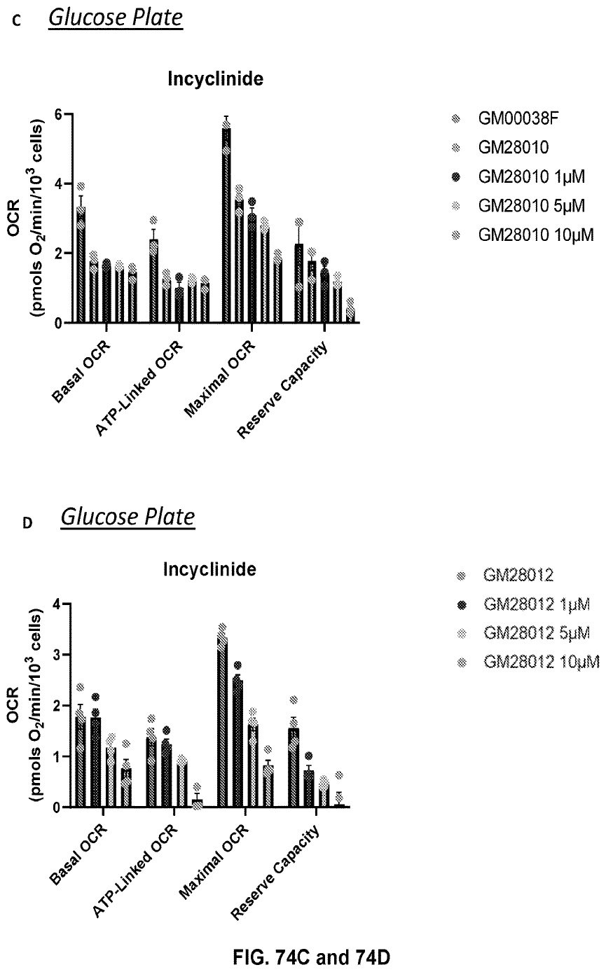

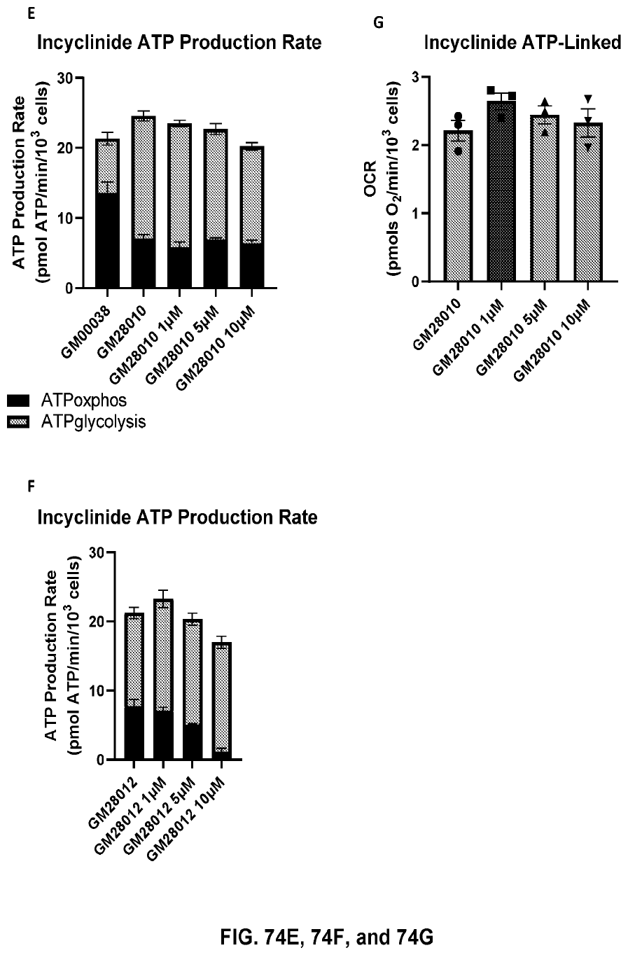

FIGS. 74A, 74B, 74C, 74D, 74E, 74F, and 74G show that treatment with incyclinide for 48 hours showed some increase to basal respiration and ATP-linked respiration in galactose-cultured cells. However, this compound showed some toxicity in glucose-cultured cells with a dose-dependent decrease in all respiratory parameters measured. Treatment with incyclinide for 48 hours showed toxicity in glucose-cultured cells with a dose-dependent decrease in all respiratory parameters measured.

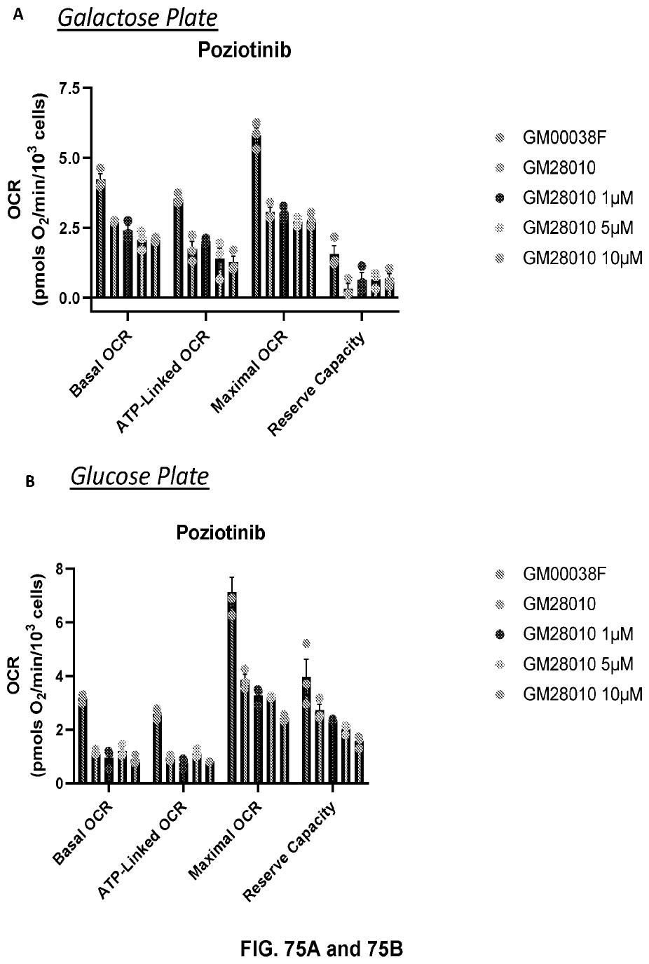

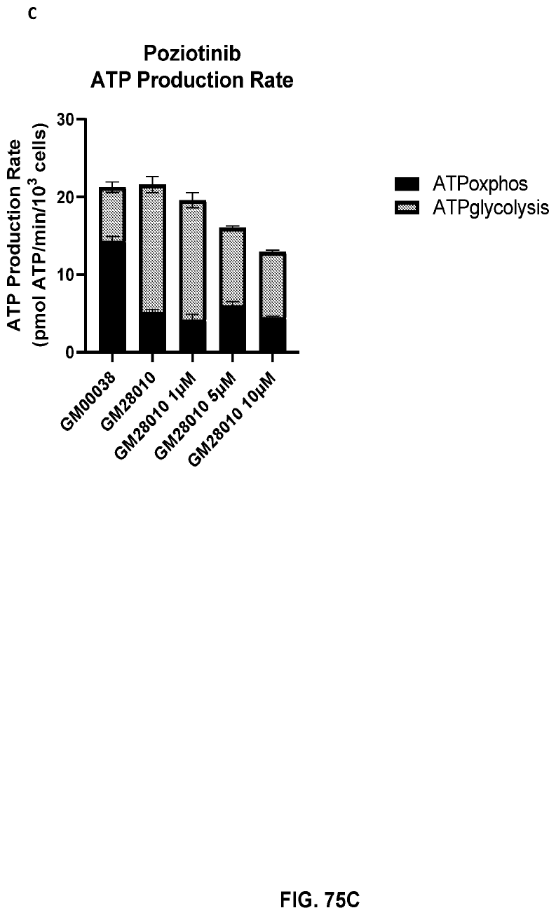

FIGS. 75A, 75B, and 75C show that treatment with poziotinib for 48 hours did not result in major changes to respiration in either glucose or galactose conditions. It did decrease ATP production through glycolysis. However, this compound showed some toxicity in glucose-cultured cells with a dose-dependent decrease in maximal respiration and glycolysis.

Although the technology herein has been described with reference to embodiments, it is to be understood that these embodiments are merely illustrative of the principles and applications of the present technology. It is therefore to be understood that numerous modifications may be made to the illustrative embodiments and that other arrangements may be devised without departing from the spirit and scope of the present technology as defined by the appended claims.

1. A method of treating Leigh Syndrome comprising administering to a patient in need thereof a therapeutically effective amount of one or more drugs, wherein the one or more repurposed drugs is an antifungal, azole antifungal, chlorinated phenol, echinocandin, lipophilic azole antifungal, statin, lysophospholipid, anionic surfactant, cationic surfactant, lipophilic antioxidant supplement, non-steroid anti-inflammatory drug, steroid, or other compound capable of treating Leigh Syndrome.

2. The method of claim 1, wherein the one or more drugs is sertaconazole.

3. The method of claim 1, wherein the one or more drugs are haloprogin, dichlorophene, atorvastatin, caspofungin, micafungin, miltefosine, sodium tetradecyl sulfate, or telapravir.

4. The method of claim 1, wherein the one or more drugs are clotrimazole, bifonazole, climbazole, fluconazole, voriconazole, tioconazole, sertaconazole, econazole, miconazole, butoconazole, oxiconazole, itraconazole, posaconazole, ketoconazole, terconazole, fenbendazole, terbinafine, amorolfine, VT-1161, haloprogin, dichlorophene, triclosan, bithinol, hexachlorophene, chloroxine, nitroxoline, benzbromarone, anidulafungin, caspofungin, micafungin, cerivastin, atorvastatin, fluvastatin, pitastatin, rosuvastatin, miltefosine, benzethonium chloride, sodium tetrdecyl sulfate, suloctidil, hexetidine, alexidine, ascorbyl palmitate, menadione, hexylresorcinol, flufenamic acid, tolfenamic acid, prasterone, pregnenolone, norgestimate, telapravir, pargyline, erythromycin, diclazuril, gliquidone, or nitazoxanide.

5. The method of claim 4, wherein the one or more drugs are clotrimazole, bifonazole, climbazole, fluconazole, voriconazole, tioconazole, sertaconazole, econazole, miconazole, butoconazole, oxiconazole, itraconazole, posaconazole, ketoconazole, terconazole, fenbendazole, terbinafine, amorolfine, or VT-1161.

6. The method of claim 4, wherein the one or more drugs are haloprogin, dichlorophene, triclosan, bithinol, hexachlorophene, chloroxine, nitroxoline, or benzbromarone.

7. The method of claim 4, wherein the one or more drugs are anidulafungin, caspofungin, or micafungin.

8. The method of claim 4, wherein the one or more drugs are cerivastin, atorvastatin, fluvastatin, pitastatin, or rosuvastatin.

9. The method of claim 4, wherein the one or more drugs are miltefosine or benzethonium chloride.

10. The method of claim 4, wherein the one or more drugs are sodium tetrdecyl sulfate or suloctidil.

11. The method of claim 4, wherein the one or more drugs are hexetidine or alexidine.

12. The method of claim 4, wherein the one or more drugs are ascorbyl palmitate, menadione, or hexylresorcinol.

13. The method of claim 4, wherein the one or more drugs are flufenamic acid or tolfenamic acid.

14. The method of claim 4, wherein the one or more drugs are prasterone, pregnenolone, or norgestimate.

15. The method of claim 4, wherein the one or more drugs are telapravir, pargyline, erythromycin, diclazuril, gliquidone, or nitazoxanide.

16. A method of treating Leigh Syndrome comprising administering to a patient in need thereof a therapeutically effective amount of one or more drugs capable of rescuing a defective SURF1 gene, wherein the one or more repurposed drugs is an antifungal, azole antifungal, chlorinated phenol, echinocandin, lipophilic azole antifungal, statin, lysophospholipid, anionic surfactant, cationic surfactant, lipophilic antioxidant supplement, non-steroid anti-inflammatory drug, steroid, or other compound capable of treating Leigh Syndrome.

17. The method of claim 16, wherein the one or more drugs is sertaconazole.

18. The method of claim 16, wherein the one or more drugs are haloprogin, dichlorophene, atorvastatin, caspofungin, micafungin, miltefosine, sodium tetradecyl sulfate, ortelapravir.

19. The method of claim 16, wherein the one or more drugs are clotrimazole, bifonazole, climbazole, fluconazole, voriconazole, tioconazole, sertaconazole, econazole, miconazole, butoconazole, oxiconazole, itraconazole, posaconazole, ketoconazole, terconazole, fenbendazole, terbinafine, amorolfine, VT-1161, haloprogin, dichlorophene, triclosan, bithinol, hexachlorophene, chloroxine, nitroxoline, benzbromarone, anidulafungin, caspofungin, micafungin, cerivastin, atorvastatin, fluvastatin, pitastatin, rosuvastatin, miltefosine, benzethonium chloride, sodium tetrdecyl sulfate, suloctidil, hexetidine, alexidine, ascorbyl palmitate, menadione, hexylresorcinol, flufenamic acid, tolfenamic acid, prasterone, pregnenolone, norgestimate, telapravir, pargyline, erythromycin, diclazuril, gliquidone, or nitazoxanide.

20. A method of restoring the functionality of complex IV in mitochondrial disorders comprising administering to a patient in need thereof a therapeutically effective amount of one or more drugs, wherein the one or more drugs are an antifungal, azole antifungal, chlorinated phenol, echinocandin, lipophilic azole antifungal, statin, lysophospholipid, anionic surfactant, cationic surfactant, lipophilic antioxidant supplement, non-steroid anti-inflammatory drug, steroid, or other compound capable of restoring the functionality of complex IV in mitochondrial disorders.

21. The method of claim 20, wherein the one or more drugs is sertaconazole.

22. The method of claim 20, wherein the one or more drugs are haloprogin, dichlorophene, atorvastatin, caspofungin, micafungin, miltefosine, sodium tetradecyl sulfate, or telapravir.

23. The method of claim 20, wherein the one or more drugs are clotrimazole, bifonazole, climbazole, fluconazole, voriconazole, tioconazole, sertaconazole, econazole, miconazole, butoconazole, oxiconazole, itraconazole, posaconazole, ketoconazole, terconazole, fenbendazole, terbinafine, amorolfine, VT-1161, haloprogin, dichlorophene, triclosan, bithinol, hexachlorophene, chloroxine, nitroxoline, benzbromarone, anidulafungin, caspofungin, micafungin, cerivastin, atorvastatin, fluvastatin, pitastatin, rosuvastatin, miltefosine, benzethonium chloride, sodium tetrdecyl sulfate, suloctidil, hexetidine, alexidine, ascorbyl palmitate, menadione, hexylresorcinol, flufenamic acid, tolfenamic acid, prasterone, pregnenolone, norgestimate, telapravir, pargyline, erythromycin, diclazuril, gliquidone, or nitazoxanide.
